(19) United States
(12) Patent Application Publication
(10) Pub. No.: US 2026/0201413 A1
(43) Pub. Date: Jul. 16, 2026

(54) IMPROVEMENT OF RECOMBINANT ADENO-ASSOCIATED VIRUS GENE THERAPY FOR HUMAN GENE THERAPY

(71) Applicant: Baylor College of Medicine, Houston, TX (US)

(72) Inventor: Alan Davis, Houston, TX (US)

(73) Assignee: Baylor College of Medicine, Houston, TX (US)

(21) Appl. No.: 19/135,275

(22) PCT Filed: Dec. 13, 2023
(86) PCT No.: PCT/US2023/083821

§ 371 Date: Jun. 3, 2025

Related U.S. Application Data
(60) Provisional application No. 63/432,645, filed on Dec. 14, 2022.

(51) Int. Cl. C12N 15/86; A61K 48/00

(52) U.S. Cl. CPC C12N 15/86; A61K 48/00; C12N 2750/14143; C12N 2750/14151

(57) ABSTRACT

Disclosed herein are new compositions and methods for production of virions. Methods and compositions comprise initial production of rep gene products, then following a suitable period of time, production of cap gene products. In certain embodiments, rep genes and/or cap genes are provided to a production host cell as RNA molecules.

### CROSS-REFERENCE TO RELATED APPLICATIONS

This application claims the benefit of priority of U.S. Provisional Patent Application No. 63/432,645 filed Dec. 14, 2022, which is hereby incorporated by reference in its entirety.

### STATEMENT OF GOVERNMENT SUPPORT

This invention was made with government support under W81XWH-21-1-0285 awarded by the U.S. Department of Defense. The government has certain rights in the invention.

### SEQUENCE LISTING

The instant application contains a Sequence Listing which has been submitted in ST26 format and is hereby incorporated by reference in its entirety. Said ST26 copy, created on Dec. 11, 2023, is named BAYM_P0382WO_Sequence_Listing.xml and is 67,887 bytes in size.

### I. TECHNICAL FIELD

This disclosure relates at least to the fields of nucleic acid research, virology, and gene therapy.

### II. BACKGROUND

### A. AAV Viral Vectors

The AAV genome is composed of a linear, single stranded DNA molecule of about 4.5 kb (e.g., about 4,680 nucleotides) that contains major open reading frames (ORF) coding for the Rep (replication), Cap (capsid) proteins, and assembly-activating protein (AAP). Flanking the AAV coding regions are two inverted terminal (ITR) repeat sequences (generally about 145 nucleotides) containing palindromic sequences that can fold over to form a self-priming hairpin structure for DNA replication. In addition, the ITR sequences are required for encapsidation of the viral genome into mature virions and, occasionally, viral integration (see e.g., Muzyczka, N., 1992, Current Topics in Microbiology & Immunology. 158, 97-129).

AAV can assume two pathways upon infection into the host cell depending on whether a helper virus is present. In the presence of a helper virus, AAV will generally enter the lytic cycle whereby the viral genome is transcribed, replicated, and encapsidated into newly formed viral particles. In the absence of helper virus functions, the AAV genome may remain as an episome or integrate as a provirus into a specific region of the host cell genome through recombination between the ITR termini and host cell sequences (see e.g., Cheung, A et al., 1980, J. Viral. 33:739-748).

The production of recombinant AAV (rAAV) particles utilizes i) a vector containing a transgene flanked by the inverted terminal repeats (ITR), which are the sole AAV cis sequences required for DNA replication, packaging, and integration, ii) the AAV replication (rep) and capsid (cap) gene products, and iii) helper gene products. In general manufacturing schemes, these components are provided in trans with one plasmid encoding AAV components, and one or more plasmids encoding helper gene products.

Transcription of the gene products encoding the three viral Cap proteins, VP1, VP2, and VP3, and the protein AAP are generally driven by the p40 promoter, while the gene products encoding the four Rep proteins, Rep78, Rep68, Rep52, and Rep40, are generally driven by the p5 and p19 promoters. Rep proteins display all enzyme functions essential for AAV DNA replication, (e.g., ITR binding, DNA helicase, and DNA site-specific nicking activity), (see e.g., Muzyczka, N., 1991, Seminars in Virology 2:281-290), and can positively and negatively regulate AAV promoters (see e.g., Labow et al., 1986, Journal of Virology 60:215-258; Pereira et al., 1997, J. Virol 71(2):1079-88; Tratschin et al., 1986, Mol. Cell Biol. 6:2884-2894) and repress numerous heterologous promoters (Antoni et al., 1991, Journal of Virology 65:396-404; Heilbronn et al., 1990, Journal of Virology 64:3012-3018; Hermonat, P. L., 1994, Cancer Letters 81:129-36; Horer, et al., 1995, Journal of Virology 69:5485-5496; and Labow et al., 1987, Molecular & Cellular Biology 7: 1320-1325).

During a lytic infection, the AAV promoters, particularly promoter p5, are transactivated by helper genes, such as the adenovirus E1A protein, and host genes, such as the YY1 protein (see e.g., Lewis, et al., 1995, J. Viral. 69:1628-1636; and Shi et al., 1991, Cell. 67:377-388). The p5 promoter driven products then positively regulate the p19 and p40 promoters, resulting in abundant production of Rep 52/40 and viral capsid proteins (see e.g., Pereira et al., 1997, J. Virol. 71(2):1079-88). Early effort to by-pass AAV rep gene regulation by substituting the p5 promoter with the SV40 early promoter failed (see e.g., Labow et al., 1988, Journal of Virology 62: 1705-1712). Instead of constitutive Rep expression, the heterologous promoter unexpectedly behaved in the same manner as the endogenous p5 promoter; repressed in the absence of, and activated in the presence of, adenovirus (see e.g., Labow et al., 1988, Journal of Virology 62:1750-1712). While these studies were the first to suggest rep repression as a mechanism for regulating heterologous promoters, these findings also implied that AAV p5 promoter driven products may be a rate-limiting factor in AAV production (see e.g., Labow et al., 1988, Journal of Virology 62: 1705-1712). Further efforts in this area have suggested that overexpression of Rep inhibits cap expression, yielding low AAV titers. In fact, overexpression of Rep78/68 has been shown to yield the lowest titer rAAV production compared to wild-type expression vectors and even vectors with attenuated Rep expression (Li et al., 1997, J. Virol. 71(7):5236-5243).

There is a continuing need in the field for improved methods and compositions for controlled and/or precise levels of AAV production genes for high titer rAAV virion production.

### B. Gene Therapy

Viral vectors are attractive tools for introducing nucleic acids into cells, as these viruses have evolved mechanisms that naturally package nucleic acids in a variety of forms, such as single stranded DNA or RNA, or double stranded DNA, for delivery to, and expression by, a host cell. The ability to introduce desired nucleic acid sequences into cells has applications in gene therapy. However, the self-replicating, integrating, and potential disease causing capabilities of many viral vectors have raised safety concerns for their use in humans.

Adeno-associated viruses (AAV) are a type of virus belonging to the Parvovirus family that have been shown to be especially well-suited for viral-based gene therapies. AAV are widely present in the human population with approximately 70-80% of the population displaying AAV-specific antibodies. AAVs are generally considered non-pathogenic, to have minimal immunogenicity, and to generally not induce inflammation (see e.g., Meier et al., 2020, Viruses 12(6):662). In addition, AAV are generally easier to produce than other viral vectors, requiring only an expression vector flanked by ITR, rep, cap, and helper genes, which are typically provided as plasmids. Unlike many other viruses, AAV has been shown to transduce or infect both dividing and differentiated, non-dividing cells with varying tropism based on capsid composition. A dozen of natural serotypes have been isolated from human and non-human primates (AAV1-12), and genetic engineering efforts have generated variations to AAV serotypes through pseudotyping and hybrid capsid generation, allowing for ever greater tissue-specificity, infectivity, and/or expression levels. Consequently, AAV is generally considered a safe and efficient vector for gene therapy.

In the year 2020, about 359 in vivo rAAV-related therapies were undergoing clinical trials, and the U.S. Food and Drug Administration predicted that 10-20 would be approved by 2025 (see e.g., Nguyen et al., 2021 Mol Ther Methods Clin Dev 21:642-655). In general, each patient typically requires a dose of about 1×10¹⁵ vector genomes (vg), i.e. mature virions packaged with a recombinant genome or expression cassette that can transduce a cell and induce expression of a transgene or therapeutic gene product, for a single whole-body treatment. Unfortunately, vg production is inefficient with typical harvests containing only about 5-30% packaged vectors that then require expensive purification procedures to eliminate empty vectors. Nguyen et al., 2021 (see e.g., Mol Ther Methods Clin Dev 21:642-655) estimated that 100,000 L of culture would be needed every year just to treat Duchenne muscular dystrophy. Given the production requirements for rAAV gene therapies and the diversifying application to treat more diseases, current rAAV production protocols are falling short of the burgeoning demand.

The large scale requirements and costs needed to produce rAAV represent a major problem in the production and adoption of rAAV as a gene therapy. Current production protocols suffer from inconsistencies, low yield, contamination, and high production costs. To overcome the aforementioned problems, there is a continuing need in the field for improved methods and compositions for rAAV production.

### III. SUMMARY

The present disclosure relates to methods and compositions for increasing the production of high titer stocks of viral particles, for example but not limited to, recombinant adeno-associated virus (rAAV), through the production and/or delivery of RNAs encoding viral components at optimal concentrations and/or at specified disparate times. In certain embodiments, methods and compositions of the disclosure generally comprise RNA (e.g., mRNA) technology to specifically and temporally control the production of AAV-production proteins. In certain embodiments, compositions and methods described herein result in unprecedent high titer viral yields and production consistency. In some embodiments, methods and compositions described herein are suitable for use in any production host cell type, for example but not limited to, mammalian or insect cells; for use in vitro, in vivo, and/or in cellulo; and for the production of one or multiple types of viral particles in the same preparation, for example but not limited to, pseudotyped or hybrid rAAV. In certain embodiments, compositions and methods of virion production described herein are suitable for research applications, for transferring genetic information into appropriate host cells, and/or for gene therapy applications for the purpose of providing therapeutic gene products, for example but not limited to, for the management and/or correction of human diseases including inherited and/or acquired diseases.

In certain embodiments, methods and compositions are provided for the production of mRNA molecules that encode viral genes. These mRNA molecules may be produced using any suitable method known in the art, for example but not limited, to in-vitro transcription, chemical synthesis, and/or in cellulo production for later extraction and/or purification. In certain embodiments the mRNA molecules are genes involved in the production of AAV, for example but not limited to, AAV replication genes (rep), capsid genes (cap), and helper genes. In certain embodiments the compositions comprise mRNA molecules synthesized to comprise modified nucleotides, for example but not limited to, pseudouridine, 5-methylcytidine, mutations in the 3′ or 5′ untranslated region (UTR), and/or circularization. In certain embodiments, methods described herein may further comprise the production of particles for transfection and/or for the delivery of mRNA to cells, for example but not limited to, lipid based formulations, calcium phosphate-mediated, and/or polyethylenimine mediated polynucleotide delivery. In certain embodiments, compositions may further comprise combinations of mRNA molecules in ratios that maximize functional viral particle production (e.g., rAAV production).

In certain embodiments, methods are provided to produce viral particles, for example but not limited, to rAAV, using mRNA and DNA compositions. In certain embodiments the viral mRNAs and a recombinant DNA vector genome (e.g. a vector genome comprising a therapeutic gene product) are co-transfected into mammalian cells. In certain embodiments, the viral mRNAs and DNA expression cassette are transfected at different time points into mammalian cells. In certain embodiments, rep, the recombinant DNA vector genome, and helper RNAs are given prior to the cap RNAs.

Certain embodiments of the present inventions are characterized through the following enumerated aspects.

Aspect 1, is a composition comprising a plurality of polynucleotides encoding one or more gene products for production of recombinant virions, wherein at least one of the plurality of polynucleotides are synthetic, and wherein gene products for the production of replication components are comprised on one or more different polynucleotides than gene products for the production of capsid components.

Aspect 2, is the composition of aspect 1, wherein the plurality of polynucleotides comprises one or more plasmids.

Aspect 3, is the composition of aspect 2, wherein the one or more plasmids comprise a promoter, one or more gene products, one or more regulatory sequence(s), inverted terminal repeats (ITRs), and/or a combination thereof.

Aspect 4, is the composition of aspect 3, wherein the one or more regulatory sequence comprises a transcription initiation sequence, an internal ribosome entry site (IRES), an enhancer, an intron, an RNA interference target sequence, a Kozak sequence, splicing regulatory elements, and/or a polyadenylation signal.

Aspect 5, is the composition of aspect 3, wherein the promoter is T7.

Aspect 6, is the composition of aspect 3, wherein the ITRs are adeno-associated virus (AAV)-derived ITRs.

Aspect 7, is the composition of aspect 1 or 3, wherein the gene products comprise Rep78, Rep68, Rep52, Rep40, VP1, VP2, VP3, E1A, E1B55K, E2A, E4orf6, assembly activating protein (AAP), viral associated RNA (VA RNA), and/or any combination thereof.

Aspect 8, is the composition of aspect 7, wherein the gene products comprise a sequence at least 80% identical to any one or more of SEQ ID NOs: 1-26 or 40-43.

Aspect 9, is the composition of aspect 7, wherein the gene products comprise a sequence according to any one or more of SEQ ID NOs: 1-26 or 40-43.

Aspect 10, is the composition of aspect 1, wherein one or more polynucleotides are ribonucleic acid molecules (RNAs) encoding gene products.

Aspect 11, is the composition of aspect 10, wherein the RNAs are produced by chemical synthesis, in vitro transcription, and/or in cellulo.

Aspect 12, is the composition of aspect 11, wherein the RNAs comprise modified nucleotides, circularization, and/or mutations relative to wild type sequences.

Aspect 13, is the composition of aspect 12, wherein the modified nucleotides comprise pseudouridine and/or 5-methylcytidine.

Aspect 14, is the composition of aspect 12, wherein the mutations comprise mutations in the 3′ and/or 5′ untranslated regions (UTRs) that protect the RNAs from degradation.

Aspect 15, is the composition of any one of aspects 10 to 14, wherein the gene products comprise Rep78, Rep68, Rep52, Rep40, VP1, VP2, VP3, E1A, E1B55K, E2A, E4orf6, assembly activating protein (AAP), viral associated RNA (VA RNA), and/or any combination thereof.

Aspect 16, is the composition of aspect 15, wherein the gene products comprise a sequence at least 80% identical to any one or more of SEQ ID NOs: 1-26 or 40-43.

Aspect 17, is the composition of aspect 15, wherein the gene products comprise a sequence according to any one or more of SEQ ID NOs: 1-26 or 40-43.

Aspect 18, is the composition of any one of aspects 10 to 17, wherein the RNAs are RNAs purified using chemical-, column-, and/or gel-based approaches.

Aspect 19, is the composition of aspect 18, wherein the RNAs are RNAs purified using high performance liquid chromatography (HPLC).

Aspect 20, is a kit comprising the composition of any one of aspects 1-19.

Aspect 21, is a composition consisting essentially of RNAs, wherein the RNAs encode gene products for AAV production.

Aspect 22, is the composition of aspect 21, wherein the RNAs encode Rep78, Rep68, Rep52, Rep40, VP1, VP2, VP3, E1A, E1B55K, E2A, E4orf6, assembly activating protein (AAP), viral associated RNA (VA RNA), and/or any combination thereof.

Aspect 23, is the composition of aspect 22, wherein the gene products comprise a sequence at least 80% identical to any one or more of SEQ ID NOs: 1-26 or 40-43.

Aspect 24, is the composition of aspect 22, wherein the gene products comprise a sequence according to any one or more of SEQ ID NOs: 1-26 or 40-43.

Aspect 25, is the composition of any one of aspects 21-24, comprising RNAs encoding Rep78, Rep68, Rep52, and/or Rep40.

Aspect 26, is the composition of aspect 25, consisting essentially of RNAs encoding Rep78, Rep68, Rep52, and/or Rep40.

Aspect 27, is the composition of any one of aspects 21-24, comprising RNAs encoding VP1, VP2, and/or VP3.

Aspect 28, is the composition of aspect 27, consisting essentially of RNAs encoding VP1, VP2, and/or VP3.

Aspect 29, is the composition of any one of aspects 21-24, comprising RNAs encoding E1A, E1B55K, E2A, and/or E4orf6.

Aspect 30, is the composition of aspect 29, consisting essentially of RNAs encoding E1A, E1B55K, E2A, and/or E4orf6.

Aspect 31, is the composition of any one of aspects 21-24, comprising RNAs encoding AAP, and/or VA RNA.

Aspect 32, is the composition of aspect 31, consisting essentially of RNAs encoding AAP and/or VA RNA.

Aspect 33, is the composition of any one of aspects 21-24, 25, or 30, comprising RNAs encoding Rep78, Rep68, Rep52, Rep40, E1A, E1B55K, E2A, and/or E4orf6.

Aspect 34, is the composition of aspect 33, consisting essentially of RNAs encoding Rep78, Rep68, Rep52, Rep40, E1A, E1B55K, E2A, and/or E4orf6.

Aspect 35, is the composition of aspect any one of aspects 21-24, 27, or 31, comprising RNAs encoding VP1, VP2, VP3, AAP and/or VA RNA.

Aspect 36, is the composition of aspect 35, consisting essentially of RNAs encoding VP1, VP2, VP3, AAP and/or VA RNA.

Aspect 37, is a composition comprising the composition of any one aspects 21 to 36, and one or more plasmids comprising a promoter sequence, one or more gene product sequence(s), one or more regulatory sequence(s), and inverted terminal repeats (ITRs).

Aspect 38, is the composition of anyone of aspects 21 to 37, wherein the RNAs are RNAs produced by chemical synthesis, in vitro transcription, and/or in cellulo.

Aspect 39, is the composition of any one of aspects 21 to 29, wherein the RNAs comprise modified nucleotides, circularization, and/or mutations relative to wild type sequences.

Aspect 40, is the composition of aspect 39, wherein the modified nucleotides comprise pseudouridine and/or 5-methylcytidine.

Aspect 41, is the composition of aspect 39, wherein the mutations comprise mutations in the 3′ or 5′ UTRs that protect the RNAs from degradation.

Aspect 42, is the composition of any one of aspects 21 to 41, wherein the RNAs are purified using chemical-, column-, and/or gel-based approaches.

Aspect 43, is the composition of anyone of aspects 21 to 42, wherein the RNAs are purified using high performance liquid chromatography (HPLC).

Aspect 44, is a kit comprising the composition of any one of aspects 21-43.

Aspect 45, is a method of producing one or more recombinant adeno-associated viruses (rAAVs) comprising performing cell transfection with the compositions of any one of aspects 1-44.

Aspect 46, is a method of producing one or more rAAVs comprising one or more temporally distinct cell transfections, wherein the temporally distinct cell transfections may comprise transfection with one or more polynucleotides.

Aspect 47, is the method of aspect 46, wherein the polynucleotides comprise a DNA-based recombinant vector, and/or RNA-based viral production genes.

Aspect 48, is the method of aspect 47, wherein the DNA-based recombinant vector genome comprises a promoter, one or more gene products, one or more regulatory sequence(s), inverted terminal repeats (ITRs), and/or any combination thereof.

Aspect 49, is the method of aspect 48, wherein the promoter is T7.

Aspect 50, is the method of aspect 48 or 49, wherein the ITRs are adeno-associated virus-derived ITRs.

Aspect 51, is the method of anyone of aspects 48 to 50, wherein the one or more regulatory sequence(s) comprises one or more of, a transcription initiation sequence, an internal ribosome entry site (IRES), an enhancer, an intron, an RNA interference target sequence, a Kozak sequence, splicing regulatory elements, and/or a polyadenylation signal.

Aspect 52, is the method of anyone of aspects 47 to 51, wherein the RNA-based viral production genes comprise rep78, rep68, rep52, rep40, VP1, VP2, VP3, E1A, E1B55K, E2A, E4orf6, AAP, VA RNA, or any combination thereof.

Aspect 53, is the method of aspect 52, wherein the RNA-based viral production genes or polypeptide encoded thereby comprise a sequence at least 80% identical to any one or more of SEQ ID NOs: 1-26 or 40-43.

Aspect 54, is the method of aspect 53, wherein the RNA-based viral production genes or polypeptide encoded thereby comprise a sequence according to any one or more of SEQ ID NOs: 1-26 or 40-43.

Aspect 55, is the method of any one of aspects 46 to 54, comprising the steps of providing to a cell: a) the DNA-based vector genome; b) rep genes, comprising rep78, rep68, rep52, rep40, and/or any combination thereof; c) viral helper genes, comprising E1A, E1B55K, E2A, E4orf6, AA P, VA RNA, and/or any combination thereof; d) cap genes, comprising VP1, VP2, and VP3, or a combination thereof; and/or e) viral helper genes, comprising AAP and/or VA RNA.

Aspect 56, is the method of aspect 55, wherein step (a), (b), and (c) occur simultaneously.

Aspect 57, is the method of aspect 55, wherein step (a), (b), and (c) occur simultaneously, and prior to step (d).

Aspect 58, is the method of aspect 55, wherein step (a), (b), and (c) occur simultaneously, and prior to step (d) and (e) which occur simultaneously.

Aspect 59, is the method of aspect 57 or 58, wherein step (d) and/or (e) occurs 1, 2, 3, 4, 5, or more days after steps (a), (b), and (c).

Aspect 60, is the method of any one of aspects 55 to 59, wherein each RNA is transfected at a concentration of about 0.1 to 10 μg/100,000 cells.

Aspect 61, is the method of any one of aspects 55 to 60, wherein each RNA is transfected at a concentration of about 0.1 to 1 μg/100,000 cells.

Aspect 62, is the method of any one of aspects 55 to 61, wherein the rep genes are transfected at a lower concentration than the viral helper genes and/or cap genes.

Aspect 63, is the method of any one of aspects 46 to 62, wherein cell transfection comprises use of calcium phosphate, cationic lipids, and polyethyleneimine (PEI), and/or any combination thereof.

Aspect 64, is the method of any one of aspects 46 to 63, wherein the transfected cells comprise mammalian and/or insect cells.

Aspect 65, are cells produced according to the method of any one of aspects 46 to 64.

Aspect 66, is the cells of aspect 65, wherein the cells are mammalian cells.

Aspect 67, is the mammalian cells of aspect 66, wherein the cells are HeLa, A549, BHK, Vero, 84-32, HEK293, and/or derivate cells thereof.

Aspect 68, is the rAAVs produced according to any one of aspects 46 to 64.

Aspect 69, is the rAAVs of aspect 68 wherein the virion comprises transcapsidation, mosaicism of the capsid, adsorption of receptor ligands, chimeric and/or hybrid capsids, and/or self-complementarity.

Aspect 70, is the rAAVs of aspect 68 or 69, wherein the AAVs are purified by clarification of cell supernatant, fractionation, HPLC, and/or any combination thereof.

Aspect 71, is a composition comprising the rAAVs of any one of aspects 68 to 70, further comprising a pharmaceutically acceptable carrier and/or excipient.

Aspect 72, is a method of providing gene therapy to an individual comprising providing the composition of aspect 71.

Aspect 73, is the method of aspect 72, wherein the rAAVs comprise an expression construct to provide an individual with a gene product in need thereof.

Aspect 74, is the method of aspect 73, wherein the gene product comprises a polypeptide, peptide, antibody, antigen fragment, ribozyme, siRNA, and/or RNAi.

Aspect 75, is the method of any one of aspects 72 to 74, wherein the compositions are provided to one or more of a cell, organ, and/or tissue of an individual in need thereof.

Aspect 76, is the method of any one of aspects 72 to 75, wherein the compositions are co-administered with one or more additional therapies.

Aspect 77, is a kit comprising, one or more compositions comprising polynucleotides, wherein the polynucleotides comprise or consisting essentially of: a) RNAs encoding Rep78, Rep68, Rep52, and/or Rep40; b) RNAs encoding E1A, E1B55K, E2A, and/or E4orf6; c) RNAs encoding Rep78, Rep68, Rep52, Rep40, E1A, E1B55K, E2A, and/or E4orf6; d) RNAs encoding VP1, VP2, and/or VP3; e) RNAs encoding AAP, and/or VA RNA; f) RNAs encoding VP1, VP2, VP3, AAP, and/or VA RNA; and/or g) one or more vector constructs comprising a promoter, one or more gene products, one or more regulatory sequence(s), inverted terminal repeats (ITRs), and/or any combination thereof.

Aspect 78, is the kit of aspect 77, wherein the RNAs comprise a sequence, or encode a polypeptide, at least 80% identical to any one or more of SEQ ID NOs: 1-26 or 40-43.

Aspect 79, is the kit of aspect 77, wherein the RNAs comprise a sequence, or encode a polypeptide, according to any one or more of SEQ ID NOs: 1-26 or 40-43.

### IV. DEFINITIONS

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention(s).

The term “effective,” as that term is used in the specification and/or claims, means adequate to accomplish a desired, expected, or intended result.

The terms “individual,” “subject,” and “patient” are used interchangeably and can refer to a human or non-human.

The terms “inhibiting” or “reducing” or “preventing” or “avoiding” or any variation of these terms, when used in the claims and/or the specification, includes any measurable decrease or complete inhibition to achieve a desired result.

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. RNA encoding the protein, from a linear or circular nature, can be produced synthetically or by in vitro methods. In particular embodiments, there are isolated nucleic acid segments and recombinant vectors incorporating nucleic acid sequences that encode a polypeptide. The term “recombinant” may be used in conjunction with a polypeptide or the name of a specific polypeptide, and this generally refers to a polypeptide produced from a nucleic acid molecule that has been manipulated in vitro or that is a replication product of such a molecule.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

The term “about” as used herein refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, ±5%, or ±1% as understood by a person of skill in the art.

The term “synthetic” or “synthesized” or any variation of these terms, as used herein refers to molecules that are designed and produced by a person skilled in the art. Methods of producing a synthetic molecule comprise chemical, in vitro, in cellulo, and/or any combination thereof. Methods of producing a synthetic molecule may further comprise selection, purification, concentration, and/or any combination thereof.

The term “helper virus” as used herein refers to viruses that when co-infected into and/or co-expressed in a host cell, supports AAV replication, AAV gene expression, and/or AAV virion production.

The term “helper gene(s),” and “helper factor(s)” are used interchangeably herein and refer to genes, RNAs, and/or proteins derived from helper viruses and/or cells that support AAV replication, AAV gene expression, and/or AAV virion production.

The term “recombinant” may be used in conjunction with a polypeptide or the name of a specific polypeptide, and this generally refers to a polypeptide produced from a nucleic acid molecule that has been manipulated in vitro or that is a replication product of such a molecule.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention(s) will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### V. DETAILED DESCRIPTION

### A. AAV Genes

The sequences of the AAV genes are reported in Srivastava, A, et al., 1983, J. Viral. 45:555-564; Muzyczka, N., 1992, Curr. Top. Micro Immunol. 158:97-129, and Ruffing, M., et al., 1992, J. Viral. 66:6922-6930. Sources for the AAV genes may include the mammalian virus serotypes AAV-1, AAV-2, AAV-3, AAV-4, AAV-5, AAV-6, AAV-7, AAV-8, AAV-9, AAV-10, AAV-11, and/or AAV-12 as well as bovine AAV and/or avian AAV. In some embodiments, the disclosure contemplates, in addition to the nucleotide sequences disclosed therein, (1) any sequence that encodes the same amino acid sequence for AAV genes shown in Srivastava, A., et al., supra, Muzyczka, N., supra, Ruffing, M., et al. supra, and/or herein; and (2) any sequence that hybridizes to the complement of the coding sequences disclosed therein and/or herein under highly stringent conditions, e.g., washing in 0.1×SSC/0.1% SDS at 68° C. (see e.g., Ausubel F. M. et al., eds., 1989, Current Protocols in Molecular Biology, Vol. I, Green Publishing Associates, Inc., and John Wiley & sons, Inc., New York, at p. 2.10.3) and encodes a functionally equivalent gene product; and/or 3) any sequence that hybridizes to the complement of the coding sequences disclosed therein and/or herein under less stringent conditions, such as moderately stringent conditions, e.g., washing in 0.2×SSC/0.1% SDS at 42° C. (see e.g., Ausubel et al., 1989, supra), yet which still encodes a functionally equivalent gene product.

Nucleic acids which encode derivatives (including fragments) and analogs of native AAV proteins can also be used in methods and/or compositions of the present disclosure, as long as such derivatives and analogs retain the ability to provide the functions required for AAV replication and production. In particular, derivatives can be made by altering genetic sequences by substitutions, additions, or deletions that provide for functionally active molecules that may have an altered phenotype. Furthermore, due to the degeneracy of nucleotide coding sequences, other sequences that encode substantially the same or a functionally equivalent AAV amino acid sequence may be used in the practice of the methods and/or compositions of the present disclosure. In certain embodiments, a gene product may contain deletions, additions or substitutions of amino acid residues within the sequence which result in silent changes thus producing a bioactive product. Such amino acid substitutions may be made on the basis of similarity in polarity, charge, solubility, hydrophobicity, hydrophilicity and/or the amphipathic nature of the residues involved. For example, negatively charged amino acids include aspartic acid and glutamic acid; positively charged amino acids include lysine and arginine; amino acids with uncharged polar head groups or nonpolar head groups having similar hydrophilicity values include the following: leucine, isoleucine, valine, glycine, alanine, asparagine, glutamine, serine, threonine, phenylalanine, tyrosine.

In addition, nucleic acids which encode derivatives and/or analogs of native AAV proteins with altered phenotypes may also be used with the methods and/or compositions of the present disclosure. In particular, in certain embodiments, alterations that decrease or increase the biological activity of the AAV proteins and/or increase or decrease the stability of the AAV proteins may be used. For example, but not limited to, nucleic acid molecules encoding temperature sensitive mutants of the AAV proteins, nucleic acid molecules encoding AAV proteins having a shortened or elongated half-life, and/or AAV proteins which are more or less susceptible to proteolytic cleavage may be used.

In certain embodiments, the concentration of compositions described herein that comprise polynucleotides encoding AAV gene products may vary according to the effect desired. In certain embodiments, it is contemplated that concentrations of polynucleotides for dosage to a host cell may be in the range from 0.1 μg/100,000 cells to 10.0 μg/100,000 cells, and that concentrations of different polynucleotides (e.g., RNAs, etc.) can affect the production of AAV titers. Thus, in some embodiments, it is contemplated that doses can be about 0.1, 0.2, 0.25, 0.3, 0.4, 0.5, 0.6, 0.7, 0.75, 0.8, 0.9, 1, 2, 3, 4, 5, 6, 7, 8, 9, or 10 μg/100,000 cells, or any range derivable therein. In some embodiments, it is contemplated that doses can be about 0.1, 0.2, 0.25, 0.3, 0.4, 0.5, 0.6, 0.7, 0.75, 0.8, 0.9, 1, 2, 3, 4, 5, 6, 7, 8, 9, or 10 μg/100,000 cells/day, or any range derivable therein. Furthermore, such doses can be administered at multiple times during a day, and/or on multiple days, weeks, or months.

In certain embodiments, methods described herein comprise transfection of host cells with 1) AAV rep RNAs, 2) transgenic/therapeutic rAAV plasmid, and 3) various adenovirus RNA(s) at day 0; followed by transfection with 4) AAV cap genes RNA, and optionally 5) RNAs encoding AAV helper proteins at days 0, 2, 3, and 5. Any of these RNAs may optionally comprise pseudouridine, be circularized, and/or be delivered using lipid nanoparticles.

In certain embodiments, methods provided herein to produce viral particles results in an increase of greater than or equal to, about or exactly, 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 100-, or 1000-fold, or any range derivable therein, relative to a control method for producing viral particles.

In certain embodiments, polynucleotides encoding AAV cap gene products transfected into a host cell 0.1, 0.2, 0.3, 0.4, 0.5, 0.6, 0.7, 0.8, 0.9, 1, 2, 3, 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, or 168 hours, or any range derivable therein, after transfection of polynucleotides encoding AAV rep gene products. In certain embodiments, polynucleotides encoding AAV cap gene products are not introduced until the replication rate and/or concentration of a transgenic rAAV has plateaued.

In certain embodiments, polynucleotides encoding AAV rep gene products are not introduced until after a host cell is transfected with a plasmid comprising an AAV ITR flanked transgene. In certain embodiments, polynucleotides encoding AAV rep gene products are transfected into a host cell 0.1, 0.2, 0.3, 0.4, 0.5, 0.6, 0.7, 0.8, 0.9, 1, 2, 3, 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, or 168 hours, or any range derivable therein, after transfection of polynucleotides comprising a transgene (e.g., a transgene flanked by AAV ITR sequences).

In certain embodiments, AAV rep gene products are cytotoxic to a host cell, and must be titered to reduce cytotoxicity. In certain embodiments, polynucleotides encoding AAV rep gene products are transfected into a host cell at a concentration lower than other gene products.

In some embodiments, AAV gene products, consist of, consist essentially of, or comprise an amino acid sequence, or is encoded by a polynucleotide sequence, having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, sequence identity to SEQ ID NOs: 1-16 or 40-43.

SEQ ID NO: 1 - Exemplary Rep78 amino acid sequence
(SEQ ID NO: 1)
MPGFYEIVIKVPSDLDEHLPGISDSFVNWVAEKEWELPPDSDMDLNLIEQAPLTVAEKLQRDFL
TEWRRVSKAPEALFFVQFEKGESYFHMHVLVETTGVKSMVLGRFLSQIREKLIQRIYRGIEPTL
PNWFAVTKTRNGAGGGNKVVDECYIPNYLLPKTQPELQWAWTNMEQYLSACLNLTERKRLVAQH
LTHVSQTQEQNKENQNPNSDAPVIRSKISARYMELVGWLVDKGITSEKQWIQEDQASYISFNAA
SNSRSQIKAALDNAGKIMSLIKTAPDYLVGQQPVEDISSNRIYKILELNGYDPQYAASVFLGWA
TKKFGKRNTIWLFGPATTGKTNIAEAIAHTVPFYGCVNWINENFPFNDCVDKMVIWWEEGKMTA
KVVESAKAILGGSKVRVDQKCKSSAQIDPTPVIVTSNINMCAVIDGNSTTFEHQQPLQDRMFKF
ELTRRLDHDFGKVTKQEVKDFFRWAKDHVVEVEHEFYVKKGGAKKRPAPSDADISEPKRVRESV
AQPSTSDAEASINYADRYQNKCSRHVGMNLMLFPCRQCERMNQNSNICFTHGQKDCLECFPVSE
SQPVSVVKKAYQKLCYIHHIMGKVPDACTACDLVNVDLDDCIFEQ
SEQ ID NO: 2 - Exemplary rep78 polynucleotide sequence
(SEQ ID NO: 2)
ATGCCGGGGTTTTACGAGATTGTGATTAAGGTCCCCAGCGACCTTGACGAGCATCTGCCCGGCA
TTTCTGACAGCTTTGTGAACTGGGTGGCCGAGAAGGAATGGGAGTTGCCGCCAGATTCTGACAT
GGATCTGAATCTGATTGAGCAGGCACCCCTGACCGTGGCCGAGAAGCTGCAGCGCGACTTTCTG
ACGGAATGGCGCCGTGTGAGTAAGGCCCCGGAGGCCCTTTTCTTTGTGCAATTTGAGAAGGGAG
AGAGCTACTTCCACATGCACGTGCTCGTGGAAACCACCGGGGTGAAATCCATGGTTTTGGGACG
TTTCCTGAGTCAGATTCGCGAAAAACTGATTCAGAGAATTTACCGCGGGATCGAGCCGACTTTG
CCAAACTGGTTCGCGGTCACAAAGACCAGAAATGGCGCCGGAGGCGGGAACAAGGTGGTGGATG
AGTGCTACATCCCCAATTACTTGCTCCCCAAAACCCAGCCTGAGCTCCAGTGGGCGTGGACTAA
TATGGAACAGTATTTAAGCGCCTGTTTGAATCTCACGGAGCGTAAACGGTTGGGTGCGCAGCAT
CTGACGCACGTGTCGCAGACGCAGGAGCAGAACAAAGAGAATCAGAATCCCAATTCTGATGCGC
CGGTGATCAGATCAAAAACTTCAGCCAGGTACATGGAGCTGGTCGGGTGGCTCGTGGACAAGGG
GATTACCTCGGAGAAGCAGTGGATCCAGGAGGACCAGGCCTCATACATCTCCTTCAATGCGGCC
TCCAACTCGCGGTCCCAAATCAAGGCTGCCTTGGACAATGCGGGAAAGATTATGAGCCTGACTA
AAACCGCCCCCGACTACCTGGTGGGCCAGCAGCCCGTGGAGGACATTTCCAGCAATCGGATTTA
TAAAATTTTGGAACTAAACGGGTACGATCCCCAATATGCGGCTTCCGTCTTTCTGGGATGGGCC
ACGAAAAAGTTCGGCAAGAGGAACACCATCTGGCTGTTTGGGCCTGCAACTACCGGGAAGACCA
ACATCGCGGAGGCCATAGCCCACACTGTGCCCTTCTACGGGTGCGTAAACTGGACCAATGAGAA
CTTTCCCTTCAACGACTGTGTCGACAAGATGGTGATCTGGTGGGAGGAGGGGAAGATGACCGCC
AAGGTCGTGGAGTCGGCCAAAGCCATTCTCGGAGGAAGCAAGGTGCGCGTGGACCAGAAATGCA
AGTCCTCGGCCCAGATAGACCCGACTCCCGTGATCGTCACCTCCAACACCAACATGTGCGCCGT
GATTGACGGGAACTCAACGACCTTCGAACACCAGCAGCCGTTGCAAGACCGGATGTTCAAATTT
GAACTCACCCGCCGTCTGGATCATGACTTTGGGAAGGTCACCAAGCAGGAAGTCAAAGACTTTT
TCCGGTGGGCAAAGGATCACGTGGTTGAGGTGGAGCATGAATTCTACGTCAAAAAGGGTGGAGC
CAAGAAAAGACCCGCCCCCAGTGACGCAGATATAAGTGAGCCCAAACGGGTGCGCGAGTCAGTT
GCGCAGCCATCGACGTCAGACGCGGAAGCTTCGATCAACTACGCAGACAGGTACCAAAACAAAT
GTTCTCGTCACGTGGGCATGAATCTGATGCTGTTTCCCTGCAGACAATGCGAGAGAATGAATCA
GAATTCAAATATCTGCTTCACTCACGGACAGAAAGACTGTTTAGAGTGCTTTCCCGTGTCAGAA
TCTCAACCCGTTTCTGTCGTCAAAAAGGCGTATCAGAAACTGTGCTACATTCATCATATCATGG
GAAAGGTGCCAGACGCTTGCACTGCCTGCGATCTGGTCAATGTGGATTTGGATGACTGCATCTT
TGAACAATAA
SEQ ID NO: 3 - Exemplary Rep68 amino acid sequence
(SEQ ID NO: 3)
MPGFYEIVIKVPSDLDEHLPGISDSFVNWVAEKEWELPPDSDMDLNLIEQAPLTVAEKLQRDFL
TEWRRVSKAPEALFFVQFEKGESYFHMHVLVETTGVKSMVLGRFLSQIREKLIQRIYRGIEPTL
PNWFAVTKTRNGAGGGNKVVDECYIPNYLLPKTQPELQWAWTNMEQYLSACLNLTERKRLVAQH
LTHVSQTQEQNKENQNPNSDAPVIRSKTSARYMELVGWLVDKGITSEKQWIQEDQASYISFNAA
SNSRSQIKAALDNAGKIMSLTKTAPDYLVGQQPVEDISSNRIYKILELNGYDPQYAASVFLGWA
TKKFGKRNTIWLFGPATTGKTNIAEAIAHTVPFYGCVNWTNENFPFNDCVDKMVIWWEEGKMTA
KVVESAKAILGGSKVRVDQKCKSSAQIDPTPVIVTSNTNMCAVIDGNSTTFEHQQPLQDRMFKF
ELTRRLDHDFGKVTKQEVKDFFRWAKDHVVEVEHEFYVKKGGAKKRPAPSDADISEPKRVRESV
AQPSTSDAEASINYADRLARGHSL
SEQ ID NO: 4 - Exemplary rep68 polynucleotide sequence
(SEQ ID NO: 4)
ATGCCGGGGTTTTACGAGATTGTGATTAAGGTCCCCAGCGACCTTGACGAGCATCTGCCCGGCA
TTTCTGACAGCTTTGTGAACTGGGTGGCCGAGAAGGAATGGGAGTTGCCGCCAGATTCTGACAT
GGATCTGAATCTGATTGAGCAGGCACCCCTGACCGTGGCCGAGAAGCTGCAGCGCGACTTTCTG
ACGGAATGGCGCCGTGTGAGTAAGGCCCCGGAGGCCCTTTTCTTTGTGCAATTTGAGAAGGGAG
AGAGCTACTTCCACATGCACGTGCTCGTGGAAACCACCGGGGTGAAATCCATGGTTTTGGGACG
TTTCCTGAGTCAGATTCGCGAAAAACTGATTCAGAGAATTTACCGCGGGATCGAGCCGACTTTG
CCAAACTGGTTCGCGGTCACAAAGACCAGAAATGGCGCCGGAGGCGGGAACAAGGTGGTGGATG
AGTGCTACATCCCCAATTACTTGCTCCCCAAAACCCAGCCTGAGCTCCAGTGGGCGTGGACTAA
TATGGAACAGTATTTAAGCGCCTGTTTGAATCTCACGGAGCGTAAACGGTTGGTGGCGCAGCAT
CTGACGCACGTGTCGCAGACGCAGGAGCAGAACAAAGAGAATCAGAATCCCAATTCTGATGCGC
CGGTGATCAGATCAAAAACTTCAGCCAGGTACATGGAGCTGGTCGGGTGGCTCGTGGACAAGGG
GATTACCTCGGAGAAGCAGTGGATCCAGGAGGACCAGGCCTCATACATCTCCTTCAATGCGGCC
TCCAACTCGCGGTCCCAAATCAAGGCTGCCTTGGACAATGCGGGAAAGATTATGAGCCTGACTA
AAACCGCCCCCGACTACCTGGTGGGCCAGCAGCCCGTGGAGGACATTTCCAGCAATCGGATTTA
TAAAATTTTGGAACTAAACGGGTACGATCCCCAATATGCGGCTTCCGTCTTTCTGGGATGGGCC
ACGAAAAAGTTCGGCAAGAGGAACACCATCTGGCTGTTTGGGCCTGCAACTACCGGGAAGACCA
ACATCGCGGAGGCCATAGCCCACACTGTGCCCTTCTACGGGTGCGTAAACTGGACCAATGAGAA
CTTTCCCTTCAACGACTGTGTCGACAAGATGGTGATCTGGTGGGAGGAGGGGAAGATGACCGCC
AAGGTCGTGGAGTCGGCCAAAGCCATTCTCGGAGGAAGCAAGGTGCGCGTGGACCAGAAATGCA
AGTCCTCGGCCCAGATAGACCCGACTCCCGTGATCGTCACCTCCAACACCAACATGTGCGCCGT
GATTGACGGGAACTCAACGACCTTCGAACACCAGCAGCCGTTGCAAGACCGGATGTTCAAATTT
GAACTCACCCGCCGTCTGGATCATGACTTTGGGAAGGTCACCAAGCAGGAAGTCAAAGACTTTT
TCCGGTGGGCAAAGGATCACGTGGTTGAGGTGGAGCATGAATTCTACGTCAAAAAGGGTGGAGC
CAAGAAAAGACCCGCCCCCAGTGACGCAGATATAAGTGAGCCCAAACGGGTGCGCGAGTCAGTT
GCGCAGCCATCGACGTCAGACGCGGAAGCTTCGATCAACTACGCAGACAGGTACCAAAACAAAT
GTTCTCGTCACGTGGGCATGAATCTGATGCTGTTTCCCTGCAGACAATGCGAGAGAATGAATCA
GAATTCAAATATCTGCTTCACTCACGGACAGAAAGACTGTTTAGAGTGCTTTCCCGTGTCAGAA
TCTCAACCCGTTTCTGTCGTCAAAAAGGCGTATCAGAAACTGTGCTACATTCATCATATCATGG
GAAAGGTGCCAGACGCTTGCACTGCCTGCGATCTGGTCAATGTGGATTTGGATGACTGCATCTT
TGAACAATAAATGATTTAAATCAGGTATGGCTGCCGATGGTTATCTTCCAGATTGGCTCGAGGA
CACTCTCTCTGA
SEQ ID NO: 40 - Exemplary rep68 amino acid sequence
(SEQ ID NO: 40)
MPGFYEIVIKVPSDLDEHLPGISDSFVNWVAEKEWELPPDSDMDLNLIEQAPLTVAEKLQRDFL
TEWRRVSKAPEALFFVQFEKGESYFHMHVLVETTGVKSMVLGRFLSQIREKLIQRIYRGIEPTL
PNWFAVTKIRNGAGGGNKVVDECYIPNYLLPKTQPELQWAWTNMEQYLSACLNLTERKRLVAQH
LTHVSQTQEQNKENQNPNSDAPVIRSKISARYMELVGWLVDKGITSEKQWIQEDQASYISFNAA
SNSRSQIKAALDNAGKIMSLTKTAPDYLVGQQPVEDISSNRIYKILELNGYDPQYAASVFLGWA
TKKFGKRNTIWLFGPATTGKINIAEAIAHTVPFYGCVNWTNENFPFNDCVDKMVIWWEEGKMTA
KVVESAKAILGGSKVRVDQKCKSSAQIDPTPVIVTSNTNMCAVIDGNSTTFEHQQPLQDRMFKF
ELTRRLDHDFGKVTKQEVKDFFRWAKDHVVEVEHEFYVKKGGAKKRPAPSDADISEPKRVRESV
AQPSTSDAEASINYADRYQNKCSRHVGMNLMLFPCRQCERMNQNSNICFTHGQKDCLECFPVSE
SQPVSVVKKAYQKLCYIHHIMGKVPDACTACDLVNVDLDDCIFEQ
SEQ ID NO: 41 - Exemplary rep68 polynucleotide sequence
(SEQ ID NO: 41)
ATGCCGGGGTTTTACGAGATTGTGATTAAGGTCCCCAGCGACCTTGACGAGCATCTGCCCGGCA
TTTCTGACAGCTTTGTGAACTGGGTGGCCGAGAAGGAATGGGAGTTGCCGCCAGATTCTGACAT
GGATCTGAATCTGATTGAGCAGGCACCCCTGACCGTGGCCGAGAAGCTGCAGCGCGACTTTCTG
ACGGAATGGCGCCGTGTGAGTAAGGCCCCGGAGGCCCTTTTCTTTGTGCAATTTGAGAAGGGAG
AGAGCTACTTCCACATGCACGTGCTCGTGGAAACCACCGGGGTGAAATCCATGGTTTTGGGACG
TTTCCTGAGTCAGATTCGCGAAAAACTGATTCAGAGAATTTACCGCGGGATCGAGCCGACTTTG
CCAAACTGGTTCGCGGTCACAAAGACCAGAAATGGCGCCGGAGGCGGGAACAAGGTGGTGGATG
AGTGCTACATCCCCAATTACTTGCTCCCCAAAACCCAGCCTGAGCTCCAGTGGGCGTGGACTAA
TATGGAACAGTATTTAAGCGCCTGTTTGAATCTCACGGAGCGTAAACGGTTGGTGGCGCAGCAT
CTGACGCACGTGTCGCAGACGCAGGAGCAGAACAAAGAGAATCAGAATCCCAATTCTGATGCGC
CGGTGATCAGATCAAAAACTTCAGCCAGGTACATGGAGCTGGTCGGGTGGCTCGTGGACAAGGG
GATTACCTCGGAGAAGCAGTGGATCCAGGAGGACCAGGCCTCATACATCTCCTTCAATGCGGCC
TCCAACTCGCGGTCCCAAATCAAGGCTGCCTTGGACAATGCGGGAAAGATTATGAGCCTGACTA
AAACCGCCCCCGACTACCTGGTGGGCCAGCAGCCCGTGGAGGACATTTCCAGCAATCGGATTTA
TAAAATTTTGGAACTAAACGGGTACGATCCCCAATATGCGGCTTCCGTCTTTCTGGGATGGGCC
ACGAAAAAGTTCGGCAAGAGGAACACCATCTGGCTGTTTGGGCCTGCAACTACCGGGAAGACCA
ACATCGCGGAGGCCATAGCCCACACTGTGCCCTTCTACGGGTGCGTAAACTGGACCAATGAGAA
CTTTCCCTTCAACGACTGTGTCGACAAGATGGTGATCTGGTGGGAGGAGGGGAAGATGACCGCC
AAGGTCGTGGAGTCGGCCAAAGCCATTCTCGGAGGAAGCAAGGTGCGCGTGGACCAGAAATGCA
AGTCCTCGGCCCAGATAGACCCGACTCCCGTGATCGTCACCTCCAACACCAACATGTGCGCCGT
GATTGACGGGAACTCAACGACCTTCGAACACCAGCAGCCGTTGCAAGACCGGATGTTCAAATTT
GAACTCACCCGCCGTCTGGATCATGACTTTGGGAAGGTCACCAAGCAGGAAGTCAAAGACTTTT
TCCGGTGGGCAAAGGATCACGTGGTTGAGGTGGAGCATGAATTCTACGTCAAAAAGGGTGGAGC
CAAGAAAAGACCCGCCCCCAGTGACGCAGATATAAGTGAGCCCAAACGGGTGCGCGAGTCAGTT
GCGCAGCCATCGACGTCAGACGCGGAAGCTTCGATCAACTACGCAGACAGGTACCAAAACAAAT
GTTCTCGTCACGTGGGCATGAATCTGATGCTGTTTCCCTGCAGACAATGCGAGAGAATGAATCA
GAATTCAAATATCTGCTTCACTCACGGACAGAAAGACTGTTTAGAGTGCTTTCCCGTGTCAGAA
TCTCAACCCGTTTCTGTCGTCAAAAAGGCGTATCAGAAACTGTGCTACATTCATCATATCATGG
GAAAGGTGCCAGACGCTTGCACTGCCTGCGATCTGGTCAATGTGGATTTGGATGACTGCATCTT
TGAACAATAA
SEQ ID NO: 5 - Exemplary Rep52 amino acid sequence
(SEQ ID NO: 5)
MELVGWLVDKGITSEKQWIQEDQASYISFNAASNSRSQIKAALDNAGKIMSLIKTAPDYLVGQQ
PVEDISSNRIYKILELNGYDPQYAASVFLGWATKKFGKRNTIWLFGPATTGKINIAEAIAHTVP
FYGCVNWTNENFPFNDCVDKMVIWWEEGKMTAKVVESAKAILGGSKVRVDQKCKSSAQIDPTPV
IVTSNINMCAVIDGNSTTFEHQQPLQDRMFKFELTRRLDHDFGKVTKQEVKDFFRWAKDHVVEV
EHEFYVKKGGAKKRPAPSDADISEPKRVRESVAQPSTSDAEASINYADRYQNKCSRHVGMNLML
FPCRQCERMNQNSNICFTHGQKDCLECFPVSESQPVSVVKKAYQKLCYIHHIMGKVPDACTACD
LVNVDLDDCIFEQ
SEQ ID NO: 6 - Exemplary rep52 polynucleotide sequence
(SEQ ID NO: 6)
ATGGAGCTGGTCGGGTGGCTCGTGGACAAGGGGATTACCTCGGAGAAGCAGTGGATCCAGGAGG
ACCAGGCCTCATACATCTCCTTCAATGCGGCCTCCAACTCGCGGTCCCAAATCAAGGCTGCCTT
GGACAATGCGGGAAAGATTATGAGCCTGACTAAAACCGCCCCCGACTACCTGGTGGGCCAGCAG
CCCGTGGAGGACATTTCCAGCAATCGGATTTATAAAATTTTGGAACTAAACGGGTACGATCCCC
AATATGCGGCTTCCGTCTTTCTGGGATGGGCCACGAAAAAGTTCGGCAAGAGGAACACCATCTG
GCTGTTTGGGCCTGCAACTACCGGGAAGACCAACATCGCGGAGGCCATAGCCCACACTGTGCCC
TTCTACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTCTCGG
AGGAAGCAAGGTGCGCGTGGACCAGAAATGCAAGTCCTCGGCCCAGATAGACCCGACTCCCGTG
ATCGTCACCTCCAACACCAACATGTGCGCCGTGATTGACGGGAACTCAACGACCTTCGAACACC
AGCAGCCGTTGCAAGACCGGATGTTCAAATTTGAACTCACCCGCCGTCTGGATCATGACTTTGG
GAAGGTCACCAAGCAGGAAGTCAAAGACTTTTTCCGGTGGGCAAAGGATCACGTGGTTGAGGTG
GAGCATGAATTCTACGTCAAAAAGGGTGGAGCCAAGAAAAGACCCGCCCCCAGTGACGCAGATA
TAAGTGAGCCCAAACGGGTGCGCGAGTCAGTTGCGCAGCCATCGACGTCAGACGCGGAAGCTTC
GATCAACTACGCAGACAGGTACCAAAACAAATGTTCTCGTCACGTGGGCATGAATCTGATGCTG
TTTCCCTGCAGACAATGCGAGAGAATGAATCAGAATTCAAATATCTGCTTCACTCACGGACAGA
AAGACTGTTTAGAGTGCTTTCCCGTGTCAGAATCTCAACCCGTTTCTGTCGTCAAAAAGGCGTA
TCAGAAACTGTGCTACATTCATCATATCATGGGAAAGGTGCCAGACGCTTGCACTGCCTGCGAT
CTGGTCAATGTGGATTTGGATGACTGCATCTTTGAACAATAA
SEQ ID NO: 7 - Exemplary Rep40 amino acid sequence
(SEQ ID NO: 7)
MELVGWLVDKGITSEKQWIQEDQASYISFNAASNSRSQIKAALDNAGKIMSLTKTAPDYLVGQQ
PVEDISSNRIYKILELNGYDPQYAASVFLGWATKKFGKRNTIWLFGPATTGKTNIAEAIAHTVP
FYGCVNWTNENFPFNDCVDKMVIWWEEGKMTAKVVESAKAILGGSKVRVDQKCKSSAQIDPTPV
IVTSNTNMCAVIDGNSTTFEHQQPLQDRMFKFELTRRLDHDFGKVTKQEVKDFFRWAKDHVVEV
EHEFYVKKGGAKKRPAPSDADISEPKRVRESVAQPSTSDAEASINYADRLARGHSL
SEQ ID NO: 8 - Exemplary rep40 polynucleotide sequence
(SEQ ID NO: 8)
ATGGAGCTGGTCGGGTGGCTCGTGGACAAGGGGATTACCTCGGAGAAGCAGTGGATCCAGGAGG
ACCAGGCCTCATACATCTCCTTCAATGCGGCCTCCAACTCGCGGTCCCAAATCAAGGCTGCCTT
GGACAATGCGGGAAAGATTATGAGCCTGACTAAAACCGCCCCCGACTACCTGGTGGGCCAGCAG
CCCGTGGAGGACATTTCCAGCAATCGGATTTATAAAATTTTGGAACTAAACGGGTACGATCCCC
AATATGCGGCTTCCGTCTTTCTGGGATGGGCCACGAAAAAGTTCGGCAAGAGGAACACCATCTG
GCTGTTTGGGCCTGCAACTACCGGGAAGACCAACATCGCGGAGGCCATAGCCCACACTGTGCCC
TTCTACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTCTCGG
AGGAAGCAAGGTGCGCGTGGACCAGAAATGCAAGTCCTCGGCCCAGATAGACCCGACTCCCGTG
ATCGTCACCTCCAACACCAACATGTGCGCCGTGATTGACGGGAACTCAACGACCTTCGAACACC
AGCAGCCGTTGCAAGACCGGATGTTCAAATTTGAACTCACCCGCCGTCTGGATCATGACTTTGG
GAAGGTCACCAAGCAGGAAGTCAAAGACTTTTTCCGGTGGGCAAAGGATCACGTGGTTGAGGTG
GAGCATGAATTCTACGTCAAAAAGGGTGGAGCCAAGAAAAGACCCGCCCCCAGTGACGCAGATA
TAAGTGAGCCCAAACGGGTGCGCGAGTCAGTTGCGCAGCCATCGACGTCAGACGCGGAAGCTTC
GATCAACTACGCAGACAGGTACCAAAACAAATGTTCTCGTCACGTGGGCATGAATCTGATGCTG
TTTCCCTGCAGACAATGCGAGAGAATGAATCAGAATTCAAATATCTGCTTCACTCACGGACAGA
AAGACTGTTTAGAGTGCTTTCCCGTGTCAGAATCTCAACCCGTTTCTGTCGTCAAAAAGGCGTA
TCAGAAACTGTGCTACATTCATCATATCATGGGAAAGGTGCCAGACGCTTGCACTGCCTGCGAT
CTGGTCAATGTGGATTTGGATGACTGCATCTTTGAACAATAAATGATTTAAATCAGGTATGGCT
GCCGATGGTTATCTTCCAGATTGGCTCGAGGACACTCTCTCTGA
SEQ ID NO: 42 - Exemplary Rep40 amino acid sequence
(SEQ ID NO: 42)
MELVGWLVDKGITSEKQWIQEDQASYISFNAASNSRSQIKAALDNAGKIMSLTKTAPDYLVGQQ
PVEDISSNRIYKILELNGYDPQYAASVFLGWATKKFGKRNTIWLFGPATTGKTNIAEAIAHTVP
FYGCVNWTNENFPFNDCVDKMVIWWEEGKMTAKVVESAKAILGGSKVRVDQKCKSSAQIDPTPV
IVTSNTNMCAVIDGNSTTFEHQQPLQDRMFKFELTRRLDHDFGKVTKQEVKDFFRWAKDHVVEV
EHEFYVKKGGAKKRPAPSDADISEPKRVRESVAQPSTSDAEASINYADRLARGHSL
SEQ ID NO: 43 - Exemplary Rep40 polynucleotide sequence
(SEQ ID NO: 43)
ATGGAGCTGGTCGGGTGGCTCGTGGACAAGGGGATTACCTCGGAGAAGCAGTGGATCCAGGAGG
ACCAGGCCTCATACATCTCCTTCAATGCGGCCTCCAACTCGCGGTCCCAAATCAAGGCTGCCTT
GGACAATGCGGGAAAGATTATGAGCCTGACTAAAACCGCCCCCGACTACCTGGTGGGCCAGCAG
CCCGTGGAGGACATTTCCAGCAATCGGATTTATAAAATTTTGGAACTAAACGGGTACGATCCCC
AATATGCGGCTTCCGTCTTTCTGGGATGGGCCACGAAAAAGTTCGGCAAGAGGAACACCATCTG
GCTGTTTGGGCCTGCAACTACCGGGAAGACCAACATCGCGGAGGCCATAGCCCACACTGTGCCC
TTCTACGGGTGCGTAAACTGGACCAATGAGAACTTTCCCTTCAACGACTGTGTCGACAAGATGG
TGATCTGGTGGGAGGAGGGGAAGATGACCGCCAAGGTCGTGGAGTCGGCCAAAGCCATTCTCGG
AGGAAGCAAGGTGCGCGTGGACCAGAAATGCAAGTCCTCGGCCCAGATAGACCCGACTCCCGTG
ATCGTCACCTCCAACACCAACATGTGCGCCGTGATTGACGGGAACTCAACGACCTTCGAACACC
AGCAGCCGTTGCAAGACCGGATGTTCAAATTTGAACTCACCCGCCGTCTGGATCATGACTTTGG
GAAGGTCACCAAGCAGGAAGTCAAAGACTTTTTCCGGTGGGCAAAGGATCACGTGGTTGAGGTG
GAGCATGAATTCTACGTCAAAAAGGGTGGAGCCAAGAAAAGACCCGCCCCCAGTGACGCAGATA
TAAGTGAGCCCAAACGGGTGCGCGAGTCAGTTGCGCAGCCATCGACGTCAGACGCGGAAGCTTC
GATCAACTACGCAGACAGATTGGCTCGAGGACACTCTCTCTGA
SEQ ID NO: 9 - Exemplary VP1 amino acid sequence
(SEQ ID NO: 9)
MAADGYLPDWLEDTLSEGIRQWWKLKPGPPPPKPAERHKDDSRGLVLPGYKYLGPFNGLDKGEP
VNEADAAALEHDKAYDRQLDSGDNPYLKYNHADAEFQERLKEDTSFGGNLGRAVFQAKKRVLEP
LGLVEEPVKTAPGKKRPVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVITTSTRTWALPTYNNHL
YKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKISADNNNSEYSWTGATKYHLNG
RDSLVNPGPAMASHKDDEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNY
NKSVNVDFTVDINGVYSEPRPIGTRYLTRNL
SEQ ID NO: 10 - Exemplary vp1 polynucleotide sequence
(SEQ ID NO: 10)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGAGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACGACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGTTCTTGAACCT
CTGGGCCTGGTTGAGGAACCTGTTAAGACGGCTCCGGGAAAAAAGAGGCCGGTAGAGCACTCTC
CTGTGGAGCCAGACTCCTCCTCGGGAACCGGAAAGGCGGGCCAGCAGCCTGCAAGAAAAAGATT
GAATTTTGGTCAGACTGGAGACGCAGACTCAGTACCTGACCCCCAGCCTCTCGGACAGCCACCA
GCAGCCCCCTCTGGTCTGGGAACTAATACGATGGCTACAGGCAGTGGCGCACCAATGGCAGACA
ATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTC
TACAAACAAATTTCCAGCCAATCAGGAGCCTCGAACGACAATCACTACTTTGGCTACAGCACCC
CTTGGGGGTATTTTGACTTCAACAGATTCCACTGCCACTTTTCACCACGTGACTGGCAAAGACT
CATCAACAACAACTGGGGATTCCGACCCAAGAGACTCAACTTCAAGCTCTTTAACATTCAAGTC
AAAGAGGTCACGCAGAATGACGGTACGACGACGATTGCCAATAACCTTACCAGCACGGTTCAGG
TGTTTACTGACTCGGAGTACCAGCTCCCGTACGTCCTCGGCTCGGCGCATCAAGGATGCCTCCC
GCCGTTCCCAGCAGACGTCTTCATGGTGCCACAGTATGGATACCTCACCCTGAACAACGGGAGT
CAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAG
CCAGAGTCTGGACCGTCTCATGAATCCTCTCATCGACCAGTACCTGTATTACTTGAGCAGAACA
AACACTCCAAGTGGAACCACCACGCAGTCAAGGCTTCAGTTTTCTCAGGCCGGAGCGAGTGACA
TTCGGGACCAGTCTAGGAACTGGCTTCCTGGACCCTGTTACCGCCAGCAGCGAGTATCAAAGAC
ATCTGCGGATAACAACAACAGTGAATACTCGTGGACTGGAGCTACCAAGTACCACCTCAATGGC
AGAGACTCTCTGGTGAATCCGGGCCCGGCCATGGCAAGCCACAAGGACGATGAAGAAAAGTTTT
TTCCTCAGAGCGGGGTTCTCATCTTTGGGAAGCAAGGCTCAGAGAAAACAAATGTGGACATTGA
AAAGGTCATGATTACAGACGAAGAGGAAATCAGGACAACCAATCCCGTGGCTACGGAGCAGTAT
GGTTCTGTATCTACCAACCTCCAGAGAGGCAACAGACAAGCAGCTACCGCAGATGTCAACACAC
AAGGCGTTCTTCCAGGCATGGTCTGGCAGGACAGAGATGTGTACCTTCAGGGGCCCATCTGGGC
AAAGATTCCACACACGGACGGACATTTTCACCCCTCTCCCCTCATGGGTGGATTCGGACTTAAA
CACCCTCCTCCACAGATTCTCATCAAGAACACCCCGGTACCTGCGAATCCTTCGACCACCTTCA
GTGCGGCAAAGTTTGCTTCCTTCATCACACAGTACTCCACGGGACAGGTCAGCGTGGAGATCGA
GTGGGAGCTGCAGAAGGAAAACAGCAAACGCTGGAATCCCGAAATTCAGTACACTTCCAACTAC
AACAAGTCTGTTAATGTGGACTTTACTGTGGACACTAATGGCGTGTATTCAGAGCCTCGCCCCA
TTGGCACCAGATACCTGACTCGTAATCTGTAA
SEQ ID NO: 11 - Exemplary VP2 amino acid sequence
(SEQ ID NO: 11)
MAPGKKRPVEHSPVEPDSSSGTGKAGQQPARKRLNFGQTGDADSVPDPQPLGQPPAAPSGLGTN
TMATGSGAPMADNNEGADGVGNSSGNWHCDSTWMGDRVITTSTRIWALPTYNNHLYKQISSQSG
ASNDNHYFGYSTPWGYFDFNRFHCHFSPRDWQRLINNNWGFRPKRLNFKLFNIQVKEVTQNDGT
TTIANNLISTVQVFTDSEYQLPYVLGSAHQGCLPPFPADVFMVPQYGYLTLNNGSQAVGRSSFY
CLEYFPSQMLRTGNNFTFSYTFEDVPFHSSYAHSQSLDRLMNPLIDQYLYYLSRTNTPSGTTTQ
SRLQFSQAGASDIRDQSRNWLPGPCYRQQRVSKTSADNNNSEYSWTGATKYHLNGRDSLVNPGP
AMASHKDDEEKFFPQSGVLIFGKQGSEKTNVDIEKVMITDEEEIRTTNPVATEQYGSVSTNLQR
GNRQAATADVNTQGVLPGMVWQDRDVYLQGPIWAKIPHTDGHFHPSPLMGGFGLKHPPPQILIK
NTPVPANPSTTFSAAKFASFITQYSTGQVSVEIEWELQKENSKRWNPEIQYTSNYNKSVNVDFT
VDINGVYSEPRPIGTRYLTRNL
SEQ ID NO: 12 - Exemplary vp2 polynucleotide sequence
(SEQ ID NO: 12)
ACGGCTCCGGGAAAAAAGAGGCCGGTAGAGCACTCTCCTGTGGAGCCAGACTCCTCCTCGGGAA
CCGGAAAGGCGGGCCAGCAGCCTGCAAGAAAAAGATTGAATTTTGGTCAGACTGGAGACGCAGA
CTCAGTACCTGACCCCCAGCCTCTCGGACAGCCACCAGCAGCCCCCTCTGGTCTGGGAACTAAT
ACGATGGCTACAGGCAGTGGCGCACCAATGGCAGACAATAACGAGGGCGCCGACGGAGTGGGTA
ATTCCTCGGGAAATTGGCATTGCGATTCCACATGGATGGGCGACAGAGTCATCACCACCAGCAC
CCGAACCTGGGCCCTGCCCACCTACAACAACCACCTCTACAAACAAATTTCCAGCCAATCAGGA
GCCTCGAACGACAATCACTACTTTGGCTACAGCACCCCTTGGGGGTATTTTGACTTCAACAGAT
TCCACTGCCACTTTTCACCACGTGACTGGCAAAGACTCATCAACAACAACTGGGGATTCCGACC
CAAGAGACTCAACTTCAAGCTCTTTAACATTCAAGTCAAAGAGGTCACGCAGAATGACGGTACG
ACGACGATTGCCAATAACCTTACCAGCACGGTTCAGGTGTTTACTGACTCGGAGTACCAGCTCC
CGTACGTCCTCGGCTCGGCGCATCAAGGATGCCTCCCGCCGTTCCCAGCAGACGTCTTCATGGT
GCCACAGTATGGATACCTCACCCTGAACAACGGGAGTCAGGCAGTAGGACGCTCTTCATTTTAC
TGCCTGGAGTACTTTCCTTCTCAGATGCTGCGTACCGGAAACAACTTTACCTTCAGCTACACTT
TTGAGGACGTTCCTTTCCACAGCAGCTACGCTCACAGCCAGAGTCTGGACCGTCTCATGAATCC
TCTCATCGACCAGTACCTGTATTACTTGAGCAGAACAAACACTCCAAGTGGAACCACCACGCAG
TCAAGGCTTCAGTTTTCTCAGGCCGGAGCGAGTGACATTCGGGACCAGTCTAGGAACTGGCTTC
CTGGACCCTGTTACCGCCAGCAGCGAGTATCAAAGACATCTGCGGATAACAACAACAGTGAATA
CTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTG
GGAAGCAAGGCTCAGAGAAAACAAATGTGGACATTGAAAAGGTCATGATTACAGACGAAGAGGA
AATCAGGACAACCAATCCCGTGGCTACGGAGCAGTATGGTTCTGTATCTACCAACCTCCAGAGA
GGCAACAGACAAGCAGCTACCGCAGATGTCAACACACAAGGCGTTCTTCCAGGCATGGTCTGGC
AGGACAGAGATGTGTACCTTCAGGGGCCCATCTGGGCAAAGATTCCACACACGGACGGACATTT
TCACCCCTCTCCCCTCATGGGTGGATTCGGACTTAAACACCCTCCTCCACAGATTCTCATCAAG
AACACCCCGGTACCTGCGAATCCTTCGACCACCTTCAGTGCGGCAAAGTTTGCTTCCTTCATCA
CACAGTACTCCACGGGACAGGTCAGCGTGGAGATCGAGTGGGAGCTGCAGAAGGAAAACAGCAA
ACGCTGGAATCCCGAAATTCAGTACACTTCCAACTACAACAAGTCTGTTAATGTGGACTTTACT
GTGGACACTAATGGCGTGTATTCAGAGCCTCGCCCCATTGGCACCAGATACCTGACTCGTAATC
TGTAA
SEQ ID NO: 13 - Exemplary VP3 amino acid sequence
(SEQ ID NO: 13)
MATGSGAPMADNNEGADGVGNSSGNWHCDSTWMGDRVITTSTRTWALPTYNNHLYKQISSQSGA
SNDNHYFGYSTPWGYFDFNRFHCHFSPRDWQRLINNNWGFRPKRLNFKLFNIQVKEVTQNDGTT
TIANNLTSTVQVFTDSEYQLPYVLGSAHQGCLPPFPADVFMVPQYGYLTLNNGSQAVGRSSFYC
LEYFPSQMLRTGNNFTFSYTFEDVPFHSSYAHSQSLDRLMNPLIDQYLYYLSRINTPSGTTTQS
RLQFSQAGASDIRDQSRNWLPGPCYRQQRVSKTSADNNNSEYSWTGATKYHLNGRDSLVNPGPA
MASHKDDEEKFFPQSGVLIFGKQGSEKTNVDIEKVMITDEEEIRTINPVATEQYGSVSTNLQRG
NRQAATADVNTQGVLPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQYTSNYNKSVNVDFTV
DTNGVYSEPRPIGTRYLTRNL
SEQ ID NO: 14 - Exemplary vp3 polynucleotide sequence
(SEQ ID NO: 14)
ATGGCTACAGGCAGTGGCGCACCAATGGCAGACAATAACGAGGGCGCCGACGGAGTGGGTAATT
CCTCGGGAAATTGGCATTGCGATTCCACATGGATGGGCGACAGAGTCATCACCACCAGCACCCG
AACCTGGGCCCTGCCCACCTACAACAACCACCTCTACAAACAAATTTCCAGCCAATCAGGAGCC
TCGAACGACAATCACTACTTTGGCTACAGCACCCCTTGGGGGTATTTTGACTTCAACAGATTCC
ACTGCCACTTTTCACCACGTGACTGGCAAAGACTCATCAACAACAACTGGGGATTCCGACCCAA
GAGACTCAACTTCAAGCTCTTTAACATTCAAGTCAAAGAGGTCACGCAGAATGACGGTACGACG
ACGATTGCCAATAACCTTACCAGCACGGTTCAGGTGTTTACTGACTCGGAGTACCAGCTCCCGT
ACGTCCTCGGCTCGGCGCATCAAGGATGCCTCCCGCCGTTCCCAGCAGACGTCTTCATGGTGCC
ACAGTATGGATACCTCACCCTGAACAACGGGAGTCAGGCAGTAGGACGCTCTTCATTTTACTGC
CTGGAGTACTTTCCTTCTCAGATGCTGCGTACCGGAAACAACTTTACCTTCAGCTACACTTTTG
AGGACGTTCCTTTCCACAGCAGCTACGCTCACAGCCAGAGTCTGGACCGTCTCATGAATCCTCT
CATCGACCAGTACCTGTATTACTTGAGCAGAACAAACACTCCAAGTGGAACCACCACGCAGTCA
AGGCTTCAGTTTTCTCAGGCCGGAGCGAGTGACATTCGGGACCAGTCTAGGAACTGGCTTCCTG
GACCCTGTTACCGCCAGCAGCGAGTATCAAAGACATCTGCGGATAACAACAACAGTGAATACTC
GTGGACTGGAGCTACCAAGTACCACCTCAATGGCAGAGACTCTCTGGTGAATCCGGGCCCGGCC
ATGGCAAGCCACAAGGACGATGAAGAAAAGTTTTTTCCTCAGAGCGGGGTTCTCATCTTTGGGA
AGCAAGGCTCAGAGAAAACAAATGTGGACATTGAAAAGGTCATGATTACAGACGAAGAGGAAAT
CAGGACAACCAATCCCGTGGCTACGGAGCAGTATGGTTCTGTATCTACCAACCTCCAGAGAGGC
AACAGACAAGCAGCTACCGCAGATGTCAACACACAAGGCGTTCTTCCAGGCATGGTCTGGCAGG
ACAGAGATGTGTACCTTCAGGGGCCCATCTGGGCAAAGATTCCACACACGGACGGACATTTTCA
CCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACAC
AGTACTCCACGGGACAGGTCAGCGTGGAGATCGAGTGGGAGCTGCAGAAGGAAAACAGCAAACG
CTGGAATCCCGAAATTCAGTACACTTCCAACTACAACAAGTCTGTTAATGTGGACTTTACTGTG
GACACTAATGGCGTGTATTCAGAGCCTCGCCCCATTGGCACCAGATACCTGACTCGTAATCTGT
AA
SEQ ID NO: 15 - Exemplary AAP amino acid sequence
(SEQ ID NO: 15)
METQTQYLTPSLSDSHQQPPLVWELIRWLQAVAHQWQTITRAPTEWVIPREIGIAIPHGWATES
SPPAPEPGPCPPTTTTSTNKFPANQEPRTTITTLATAPLGGILTSTDSTATFHHVTGKDSSTTT
GDSDPRDSTSSSLTFKSKRSRRMTVRRRLPITLPARFRCLLTRSTSSRTSSARRIKDASRRSQQ
TSSWCHSMDTSP
SEQ ID NO: 16 - Exemplary AAP polynucleotide sequence
(SEQ ID NO: 16)
CTGGAGACGCAGACTCAGTACCTGACCCCCAGCCTCTCGGACAGCCACCAGCAGCCCCCTCTGG
TCTGGGAACTAATACGATGGCTACAGGCAGTGGCGCACCAATGGCAGACAATAACGAGGGCGCC
GACGGAGTGGGTAATTCCTCGGGAAATTGGCATTGCGATTCCACATGGATGGGCGACAGAGTCA
TCACCACCAGCACCCGAACCTGGGCCCTGCCCACCTACAACAACCACCTCTACAAACAAATTTC
CAGCCAATCAGGAGCCTCGAACGACAATCACTACTTTGGCTACAGCACCCCTTGGGGGTATTTT
GACTTCAACAGATTCCACTGCCACTTTTCACCACGTGACTGGCAAAGACTCATCAACAACAACT
GGGGATTCCGACCCAAGAGACTCAACTTCAAGCTCTTTAACATTCAAGTCAAAGAGGTCACGCA
GAATGACGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCCCAGCAG
ACGTCTTCATGGTGCCACAGTATGGATACCTCACCCTGA

### B. Helper Factors

Helper viruses for AAV replication, include but are not limited to herpesvirus family (for example, but not limited to, HSV-1, HSV-2, VIV, HHV6, and human cytomegalovirus), adenovirus (for example, but not limited to, hAdV5, hAdV2), papillomaviruses (for example, but not limited to, HPV-16), baculovirus, parvovirus (for example, but not limited to, human bocavirus 1 (hBoV1), hBoV2-4, and gorilla bocavirus), and/or varicella zoster virus (for example, but not limited to, VZV). Helper viruses for use with the present disclosure can be any virus that induces and/or supports AAV replication.

Helper factors for AAV replication can derive from one or more helper viruses and can be used alone or in combination. Examples of helper factors include, but are not limited to, HSV-1 HP complex, single stranded DNA (ssDNA) binding protein ICP8, ICPO, ICP4, ICP22, HSV1 polymerase, E1A, E2A, E1B55K, E4orf6 (or E4orf3), virus-associated RNAs 1 and 11 (VA RNA), Protein IX, and/or E1B19K. Other helper factors not listed here, but known by one skilled in the art to induce and/or support AAV replication are considered within the scope of the present disclosure.

In certain embodiments adenovirus helper factors are used and can include for example, but not limited to, E1A, E2A, E1B55K, VA RNA, E4A, E4orf6, E4orf3, Protein IX, E1B19K, and/or any combinations thereof. In certain embodiments, adenovirus helper factors are E1A, E2A, E1B55K, and/or E4orf6 (or E4orf3). In certain embodiments, E1A and E2A activate p5 and p19 to induce expression of Rep genes. E2A is a ssDNA binding protein that, in certain embodiments, promotes AAV DNA replication, participates in splicing along with Rep proteins, mRNA processing and export, and capsid production. In certain embodiments, E1B55K and E4orf6 complex to facilitate second strand synthesis and DNA replication. In certain embodiments, both E1B55K and E4orf6 also participate in AAV mRNA export to promote AAV gene expression. In certain embodiments, E4orf6 can degrade Mre11, a component of the MRN complex that recognizes double stranded breaks and can initiate the DNA damage response; MRN is known to limit AAV transduction and replication. In certain embodiments, E4orf6 can mediate degradation of de novo-assembled AAV particles and Rep52 via ubiquitination. Virus-associated RNAs (VA RNA) are non-coding RNA transcripts from adenovirus, of which at least two, VA RNA_{I/II}, in some embodiments, can act as helper factors by inhibiting the cellular innate immune protein double-stranded RNA-activated kinase (PKR) response, thereby preventing shutdown of general translation during viral infection and ensuring enhance expression of viral proteins.

In certain embodiments, the concentration of compositions described herein that comprise helper factors may vary according to the effect desired. In certain embodiments, it is contemplated that concentrations of polynucleotides for dosage to a host cell may be in the range from 0.1 μg/100,000 cells to 10.0 μg/100,000 cells, and that concentrations of different polynucleotides (e.g., RNAs, etc.) can affect the production of AAV titers. Thus, in some embodiments, it is contemplated that doses can be about 0.1, 0.2, 0.25, 0.3, 0.4, 0.5, 0.6, 0.7, 0.75, 0.8, 0.9, 1, 2, 3, 4, 5, 6, 7, 8, 9, or 10 μg/100,000 cells, or any range derivable therein. In some embodiments, it is contemplated that doses can be about 0.1, 0.2, 0.25, 0.3, 0.4, 0.5, 0.6, 0.7, 0.75, 0.8, 0.9, 1, 2, 3, 4, 5, 6, 7, 8, 9, or 10 μg/100,000 cells/day, or any range derivable therein. Furthermore, such doses can be administered at multiple times during a day, and/or on multiple days, weeks, or months.

In some embodiments, a helper factor gene products consist of, consist essentially of, or comprise an amino acid sequence, or is encoded by a polynucleotide sequence, having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identity to SEQ ID NOs: 17 to 26.

SEQ ID NO: 17 - Exemplary E1A amino acid sequence
(SEQ ID NO: 17)
MRHIICHGGVITEEMAASLLDQLIEEVLADNLPPPSHFEPPTLHELYDLDVTAPEDPNEEAVSQ
IFPESVMLAVQEGIDLFTFPPAPGSPEPPHLSRQPEQPEQRALGPVSMPNLVPEVIDLTCHEAG
FPPSDDEDEEGEEFVLDYVEHPGHGCRSCHYHRRNTGDPDIMCSLCYMRTCGMFVYSPVSEPEP
EPEPEPEPARPTRRPKLVPAILRRPTSPVSRECNSSTDSCDSGPSNTPPEIHPVVPLCPIKPVA
VRVGGRRQAVECIEDLLNESGQPLDLSCKRPRP
SEQ ID NO: 18 - Exemplary E1A polynucleotide sequence
(SEQ ID NO: 18)
ATGAGACATATTATCTGCCACGGAGGTGTTATTACCGAAGAAATGGCCGCCAGTCTTTTGGACC
AGCTGATCGAAGAGGTACTGGCTGATAATCTTCCACCTCCTAGCCATTTTGAACCACCTACCCT
TCACGAACTGTATGATTTAGACGTGACGGCCCCCGAAGATCCCAACGAGGAGGCGGTTTCGCAG
ATTTTTCCCGAGTCTGTAATGTTGGCGGTGCAGGAAGGGATTGACTTATTCACTTTTCCGCCGG
CGCCCGGTTCTCCGGAGCCGCCTCACCTTTCCCGGCAGCCCGAGCAGCCGGAGCAGAGAGCCTT
GGGTCCGGTTTCTATGCCAAACCTTGTGCCGGAGGTGATCGATCTTACCTGCCACGAGGCTGGC
TTTCCACCCAGTGACGACGAGGATGAAGAGGGTGAGGAGTTTGTGTTAGATTATGTGGAGCACC
CCGGGCACGGTTGCAGGTCTTGTCATTATCACCGGAGGAATACGGGGGACCCAGATATTATGTG
TTCGCTTTGCTATATGAGGACCTGTGGCATGTTTGTCTACAGTAAGTGAAAATTATGGGCAGTC
GGTGATAGAGTGGTGGGTTTGGTGTGGTAATTTTTTTTTAATTTTTACAGTTTTGTGGTTTAAA
GAATTTTGTATTGTGATTTTTTAAAAGGTCCTGTGTCTGAACCTGAGCCTGAGCCCGAGCCAGA
ACCGGAGCCTGCAAGACCTACCCGGCGTCCTAAATTGGTGCCTGCTATCCTGAGACGCCCGACA
TCACCTGTGTCTAGAGAATGCAATAGTAGTACGGATAGCTGTGACTCCGGTCCTTCTAACACAC
CTCCTGAGATACACCCGGTGGTCCCGCTGTGCCCCATTAAACCAGTTGCCGTGAGAGTTGGTGG
GCGTCGCCAGGCTGTGGAATGTATCGAGGACTTGCTTAACGAGTCTGGGCAACCTTTGGACTTG
AGCTGTAAACGCCCCAGGCCATAA
SEQ ID NO: 19 - Exemplary E1B55K amino acid sequence
(SEQ ID NO: 19)
MERRNPSERGVPAGFSGHASVESGGETQESPATVVFRPPGNNTDGGATAGGSQAAAAAGAEPME
PESRPGPSGMNVVQVAELFPELRRILTINEDGQGLKGVKRERGASEATEEARNLTFSLMTRHRP
ECVTFQQIKDNCANELDLLAQKYSIEQLTTYWLQPGDDFEEAIRVYAKVALRPDCKYKISKLVN
IRNCCYISGNGAEVEIDTEDRVAFRCSMINMWPGVLGMDGVVIMNVRFTGPNFSGTVFLANTNL
ILHGVSFYGFNNTCVEAWTDVRVRGCAFYCCWKGVVCRPKSRASIKKCLFERCTLGILSEGNSR
VRHNVASDCGCFMLVKSVAVIKHNMVCGNCEDRASQMLTCSDGNCHLLKTIHVASHSRKAWPVF
EHNILTRCSLHLGNRRGVFLPYQCNLSHTKILLEPESMSKVNLNGVFDMTMKIWKVLRYDETRT
RCRPCECGGKHIRNQPVMLDVTEELRPDHLVLACTRAEFGSSDEDTD
SEQ ID NO: 20 - Exemplary E1B55K polynucleotide sequence
(SEQ ID NO: 20)
ATGGAGCGAAGAAACCCATCTGAGCGGGGGGTACCTGCTGGATTTTCTGGCCATGCATCTGTGG
AGAGCGGTGGTGAGACACAAGAATCGCCTGCTACTGTTGTCTTCCGTCCGCCCGGCAATAATAC
CGACGGAGGAGCAACAGCAGGAGGAAGCCAGGCGGCGGCGGCGGCAGGAGCAGAGCCCATGGAA
CCCGAGAGCCGGCCTGGACCCTCGGGAATGAATGTTGTACAGGTGGCTGAACTGTTTCCAGAAC
TGAGACGCATTTTAACCATTAACGAGGATGGGCAGGGGCTAAAGGGGGTAAAGAGGGAGCGGGG
GGCTTCTGAGGCTACAGAGGAGGCTAGGAATCTAACTTTTAGCTTAATGACCAGACACCGTCCT
GAGTGTGTTACTTTTCAGCAGATTAAGGATAATTGCGCTAATGAGCTTGATCTGCTGGCGCAGA
AGTATTCCATAGAGCAGCTGACCACTTACTGGCTGCAGCCAGGGGATGATTTTGAGGAGGCTAT
TAGGGTATATGCAAAGGTGGCACTTAGGCCAGATTGCAAGTACAAGATTAGCAAACTTGTAAAT
ATCAGGAATTGTTGCTACATTTCTGGGAACGGGGCCGAGGTGGAGATAGATACGGAGGATAGGG
TGGCCTTTAGATGTAGCATGATAAATATGTGGCCGGGGGTGCTTGGCATGGACGGGGTGGTTAT
TATGAATGTGAGGTTTACTGGTCCCAATTTTAGCGGTACGGTTTTCCTGGCCAATACCAATCTT
ATCCTACACGGTGTAAGCTTCTATGGGTTTAACAATACCTGTGTGGAAGCCTGGACCGATGTAA
GGGTTCGGGGCTGTGCCTTTTACTGCTGCTGGAAGGGGGTGGTGTGTCGCCCCAAAAGCAGGGC
TTCAATTAAGAAATGCCTGTTTGAAAGGTGTACCTTGGGTATCCTGTCTGAGGGTAACTCCAGG
GTGCGCCACAATGTGGCCTCCGACTGTGGTTGCTTCATGCTAGTGAAAAGCGTGGCTGTGATTA
AGCATAACATGGTGTGTGGCAACTGCGAGGACAGGGCCTCTCAGATGCTGACCTGCTCGGACGG
CAACTGTCACTTGCTGAAGACCATTCACGTAGCCAGCCACTCTCGCAAGGCCTGGCCAGTGTTT
GAGCACAACATACTGACCCGCTGTTCCTTGCATTTGGGTAACAGGAGGGGGGTGTTCCTACCTT
ACCAATGCAATTTGAGTCACACTAAGATATTGCTTGAGCCCGAGAGCATGTCCAAGGTGAACCT
GAACGGGGTGTTTGACATGACCATGAAGATCTGGAAGGTGCTGAGGTACGATGAGACCCGCACC
AGGTGCAGACCCTGCGAGTGTGGCGGTAAACATATTAGGAACCAGCCTGTGATGCTGGATGTGA
CCGAGGAGCTGAGGCCCGATCACTTGGTGCTGGCCTGCACCCGCGCTGAGTTTGGCTCTAGCGA
TGAAGATACAGATTGA
SEQ ID NO: 21 - Exemplary E2A amino acid sequence
(SEQ ID NO: 21)
MASREEEQRETTPERGRGAARRPPTMEDVSSPSPSPPPPRAPPKKRLRRRLESEDEEDSSQDAL
VPRTPSPRPSTSTADLAIASKKKKKRPSPKPERPPSPEVIVDSEEEREDVALQMVGFSNPPVLI
KHGKGGKRTVRRLNEDDPVARGMRTQEEKEESSEAESESTVINPLSLPIVSAWEKGMEAARALM
DKYHVDNDLKANFKLLPDQVEALAAVCKTWLNEEHRGLQLTFTSNKTFVTMMGRFLQAYLQSFA
EVTYKHHEPTGCALWLHRCAEIEGELKCLHGSIMINKEHVIEMDVISENGQRALKEQSSKAKIV
KNRWGRNVVQISNTDARCCVHDAACPANQFSGKSCGMFFSEGAKAQVAFKQIKAFMQALYPNAQ
TGHGHLLMPLRCECNSKPGHAPFLGRQLPKLTPFALSNAEDLDADLISDKSVLASVHHPALIVF
QCCNPVYRNSRAQGGGPNCDFKISAPDLLNALVMVRSLWSENFTELPRMVVPEFKWSTKHQYRN
VSLPVAHSDARQNPFDF
SEQ ID NO: 22 - Exemplary E2A polynucleotide sequence
(SEQ ID NO: 22)
ATGGCCAGTCGGGAAGAGGAGCAGCGCGAAACCACCCCCGAGCGCGGACGCGGTGCGGCGCGAC
GTCCACCAACCATGGAGGACGTGTCGTCCCCGTCGCCGTCGCCGCCGCCTCCCCGCGCGCCCCC
AAAAAAGCGGCTGAGGCGGCGTCTCGAGTCCGAGGACGAAGAAGACTCGTCACAAGATGCGCTG
GTGCCGCGCACACCCAGCCCGCGGCCATCGACCTCGACGGCGGATTTGGCCATTGCGTCCAAAA
AGAAAAAGAAGCGCCCCTCTCCCAAGCCCGAGCGCCCGCCATCCCCAGAGGTGATCGTGGACAG
CGAGGAAGAAAGAGAAGATGTGGCGCTACAAATGGTGGGTTTCAGCAACCCACCGGTGCTAATC
AAGCACGGCAAGGGAGGTAAGCGCACGGTGCGGCGGCTGAATGAAGACGACCCAGTGGCGCGGG
GTATGCGGACGCAAGAGGAAAAGGAAGAGTCCAGTGAAGCGGAAAGTGAAAGCACGGTGATAAA
CCCGCTGAGCCTGCCGATCGTGTCTGCGTGGGAGAAGGGCATGGAGGCTGCGCGCGCGTTGATG
GACAAGTACCACGTGGATAACGATCTAAAGGCAAACTTCAAGCTACTGCCTGACCAAGTGGAAG
CTCTGGCGGCCGTATGCAAGACCTGGCTAAACGAGGAGCACCGCGGGTTGCAGCTGACCTTCAC
CAGCAACAAGACCTTTGTGACGATGATGGGGCGATTCCTGCAGGCGTACCTGCAGTCGTTTGCA
GAGGTAACCTACAAGCACCACGAGCCCACGGGCTGCGCGTTGTGGCTGCACCGCTGCGCTGAGA
TCGAAGGCGAGCTTAAGTGTCTACACGGGAGCATTATGATAAATAAGGAGCACGTGATTGAAAT
GGATGTGACGAGCGAAAACGGGCAGCGCGCGCTGAAGGAGCAGTCTAGCAAGGCCAAGATCGTG
AAGAACCGGTGGGGCCGAAATGTGGTGCAGATCTCCAACACCGACGCAAGGTGCTGCGTGCATG
ACGCGGCCTGTCCGGCCAATCAGTTTTCCGGCAAGTCTTGCGGCATGTTCTTCTCTGAAGGCGC
AAAGGCTCAGGTGGCTTTTAAGCAGATCAAGGCTTTCATGCAGGCGCTGTATCCTAACGCCCAG
ACCGGGCACGGTCACCTTCTGATGCCACTACGGTGCGAGTGCAACTCAAAGCCTGGGCATGCAC
CCTTTTTGGGAAGGCAGCTACCAAAGTTGACTCCGTTCGCCCTGAGCAACGCGGAGGACCTGGA
CGCGGATCTGATCTCCGACAAGAGCGTGCTGGCCAGCGTGCACCACCCGGCGCTGATAGTGTTC
CAGTGCTGCAACCCTGTGTATCGCAACTCGCGCGCGCAGGGCGGAGGCCCCAACTGCGACTTCA
AGATATCGGCGCCCGACCTGCTAAACGCGTTGGTGATGGTGCGCAGCCTGTGGAGTGAAAACTT
CACCGAGCTGCCGCGGATGGTTGTGCCTGAGTTTAAGTGGAGCACTAAACACCAGTATCGCAAC
GTGTCCCTGCCAGTGGCGCATAGCGATGCGCGGCAGAACCCCTTTGATTTTTAA
SEQ ID NO: 23 - Exemplary E4orf6 amino acid sequence
(SEQ ID NO: 23)
MTTSGVPFGMTLRPTRSRLSRRTPYSRDRLPPFETETRATILEDHPLLPECNTLTMHNAWTSPS
PPVEQPQVGQQPVAQQLDSDMNLSELPGEFINITDERLARQETVWNITPKNMSVTHDMMLFKAS
RGERTVYSVCWEGGGRLNTRVL
SEQ ID NO: 24 - Exemplary E4orf6 polynucleotide sequence
(SEQ ID NO: 24)
ATGACTACGTCCGGCGTTCCATTTGGCATGACACTACGACCAACACGATCTCGGTTGTCTCGGC
GCACTCCGTACAGTAGGGATCGCCTACCTCCTTTTGAGACAGAGACCCGCGCTACCATACTGGA
GGATCATCCGCTGCTGCCCGAATGTAACACTTTGACAATGCACAACGTGAGTTACGTGCGAGGT
CTTCCCTGCAGTGTGGGATTTACGCTGATTCAGGAATGGGTTGTTCCCTGGGATATGGTTCTGA
CGCGGGAGGAGCTTGTAATCCTGAGGAAGTGTATGCACGTGTGCCTGTGTTGTGCCAACATTGA
TATCATGACGAGCATGATGATCCATGGTTACGAGTCCTGGGCTCTCCACTGTCATTGTTCCAGT
CCCGGTTCCCTGCAGTGCATAGCCGGCGGGCAGGTTTTGGCCAGCTGGTTTAGGATGGTGGTGG
ATGGCGCCATGTTTAATCAGAGGTTTATATGGTACCGGGAGGTGGTGAATTACAACATGCCAAA
AGAGGTAATGTTTATGTCCAGCGTGTTTATGAGGGGTCGCCACTTAATCTACCTGCGCTTGTGG
TATGATGGCCACGTGGGTTCTGTGGTCCCCGCCATGAGCTTTGGATACAGCGCCTTGCACTGTG
GGATTTTGAACAATATTGTGGTGCTGTGCTGCAGTTACTGTGCTGATTTAAGTGAGATCAGGGT
GCGCTGCTGTGCCCGGAGGACAAGGCGTCTCATGCTGCGGGCGGTGCGAATCATCGCTGAGGAG
ACCACTGCCATGTTGTATTCCTGCAGGACGGAGCGGCGGCGGCAGCAGTTTATTCGCGCGCTGC
TGCAGCACCACCGCCCTATCCTGATGCACGATTATGACTCTACCCCCATGTAGGCGTGGACTTC
CCCTTCGCCGCCCGTTGAGCAACCGCAAGTTGGACAGCAGCCTGTGGCTCAGCAGCTGGACAGC
GACATGAACTTAAGCGAGCTGCCCGGGGAGTTTATTAATATCACTGATGAGCGTTTGGCTCGAC
AGGAAACCGTGTGGAATATAACACCTAAGAATATGTCTGTTACCCATGATATGATGCTTTTTAA
GGCCAGCCGGGGAGAAAGGACTGTGTACTCTGTGTGTTGGGAGGGAGGTGGCAGGTTGAATACT
AGGGTTCTGTGA
SEQ ID NO: 25 - Exemplary VA_{I} polynucleotide sequence
(SEQ ID NO: 25)
GGGCACTCTTCCGTGGTCTGGTGGATAAATTCGCAAGGGTATCATGGCGGACGACCGGGGTTCG
AACCCCGGATCCGGCCGTCCGCCGTGATCCATGCGGTTACCGCCCGCGTGTCGAACCCAGGTGT
GCGACGTCAGACAACGGGGGAGCGCTCCT
SEQ ID NO: 26 - Exemplary VA_{I} polynucleotide sequence
(SEQ ID NO: 26)
GGCTCGCTCCCTGTAGCCGGAGGGTTATTTTCCAAGGGTTGAGTCGCAGGACCCCCGGTTCGAG
TCTCGGGCCGGCCGGACTGCGGCGAACGGGGGTTTGCCTCCCCGTCATGCAAGACCCCGCTTGC
AAATTCCTCCGGAAACAGGGACGAGCCCCT

### C. Expression Systems

A variety of host-expression vector systems may be utilized to express genes for rAAV production. The expression systems that may be used for purposes of the immediate disclosure may include, but are not limited to, mammalian cell systems (for example, but not limited to, COS cell lines, CHO cell lines, BHK cell lines, 293 cell lines, 3T3 cell lines, etc.) harboring recombinant expression constructs containing promoters derived from the genome of mammalian cells (for example, but not limited to, metallothionein promoter) or from mammalian viruses (for example, but not limited to, the adenovirus late promoter, or the vaccinia virus 7.5 K promoter). In certain embodiments, promoters to express AAV gene products within a cell line may be drawn from those that are highly regulated within the host cell. In some embodiments, inducible gene regulation may be achieved using simple inducible promoter systems, including but not limited to, the metallothionine promoter (MT) and heat shock promoter, or by using the mouse mammary tumor virus promoter (MMTV) which is responsive to glucocorticoid stimulation. Alternatively and/or additionally, in some embodiments, a more flexible, though more complex inducible regulation system can be achieved through a “binary” gene approach. These binary regulation systems utilize a trans-activator gene product to control expression of a second gene of interest. In addition, in certain embodiments, repressor based binary systems may be used to regulate gene expression (see e.g., Hu et al., 1987, Cell 48:555-566; and Brown et al., 1987, Cell 49:603-612). For example, the tetR system utilizes the bacterial repressor tetR and insertion of tetR operator sequences in the promoter region of a gene of interest. Induction of gene expression in such a system involves the application of an inducer molecule that binds to and inactivates the repressor molecule resulting in activation of gene expression.

In some embodiments, the coding region of any AAV production genes may be linked to any number of promoters in an expression vector that can be activated in the chosen system, for example but not limited to, in cellulo expression. Additionally and/or alternatively, in some embodiments, an expression vector may contain a selectable marker so that cells receiving the vector may be identified. In certain embodiments, selectable markers and their attendant selection agents can be drawn from the group including but not limited to aminoglycoside phosphotransferase/G418, hygromycin-B phosphotransferase/hygromycin-B, and amplifiable selection markers such as dihydrofolate reductase/methotrexate and others known to skilled practitioners. Additionally and/or alternatively, in some embodiments, an expression vector may contain a reporter so that cells receiving the vector may be identified. Reporters may be any fluorescent, colorimetric, or light-sensitive proteins known in the art.

In some embodiments, specific initiation signals are also required for initiation of translation of inserted protein coding sequences. These initiation signals include the ATG initiation codon and adjacent sequences. In certain embodiments, exogenous translational control signals and initiation sequences can be of a variety of origins, both natural and/or synthetic. For example, but not limited to, E. coli expression vectors can contain translational control sequences, such as an appropriately positioned ribosome binding site and initiation ATG.

Expression of the AAV gene products may be controlled at the level of translation by the replacement of the ATG start codon with the less efficient ACG codon resulting in a decrease in the production of the desired protein. Conversely, in some embodiments, coding sequences may be codon optimized to increase translation efficiency and subsequent virion production. In addition, in some embodiments, the 5′ end of the gene may be genetically engineered to contain specific nucleotide sequences to which translation repressor proteins bind (see e.g., Melefors, 1993, Bioessays 15:85-90). In some embodiments, binding of a translation repressor protein to an mRNA molecule decreases the translation of the mRNA. In some embodiments, utilizing such a system, the levels of protein may be controlled by regulating the level or activity of the translational repressor protein. In some embodiments, such sequences include, but are not limited to, sequences such as the iron-response element. The iron response element folds into a stem-loop structure that binds a translation repressor protein called aconitase which blocks the translation of any RNA sequence downstream. Aconitase is an iron-binding protein, and exposure of the cell to iron causes it to dissociate from the RNA, releasing the block to translation. Therefore, in some embodiments, modification of the 5′ end of the desired gene to include the iron responsive element provides a system for selectively and efficiently inducing the expression of the desired protein by exposing cells to iron.

In some embodiments, translation of an mRNA may also be controlled taking advantage of a translational process referred to as translational recoding. In such a process, a specific recoding signal in the mRNA molecule causes the growing polypeptide chain occasionally to slip backward by one nucleotide on the ribosome as translation proceeds. The ribosome then resumes translation in a new reading frame resulting in production of a truncated protein. In some embodiments, a recoding signal sequence, which consists of the nucleotides UUUUUUA, may be included to produce the desired level of expression.

In some embodiments, level of expression of a gene product may also be controlled by altering the stability of an mRNA. More specifically, in some embodiments, the half-life of an mRNA may be significantly decreased by including, or increased by excluding, specific sequences known to stimulate RNA degradation. For example, in some embodiments, the half-life of an mRNA may be significantly decreased by cloning of a sequence rich in A and U nucleotides in the 3′ untranslated (UTR). In some embodiments, such an AU-rich sequence accelerates mRNA degradation. In addition, in some embodiments, mRNAs containing recognition sites in their 3′ UTR for specific endonucleases that cleave RNA may be generated or avoided. In some embodiments, use of such sequences can result in refined control of gene expression.

### D. RNA, MRNA

RNA(s) is the usual abbreviation for ribonucleic acid(s). It is a nucleic acid molecule, that is, a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, that is, ribose, of a first and a phosphate moiety of a second, adjacent monomer. The specific order of the monomers, that is, the order of the bases linked to the sugar/phosphate-backbone, is called the RNA sequence. Usually RNA may be obtainable by transcription of a DNA sequence, for example, but not limited to, inside a cell. In eukaryotic cells, transcription is typically performed inside the nucleus or the mitochondria. In vivo transcription of DNA usually results in the so-called premature RNA which has to be processed into so-called messenger-RNA, usually abbreviated as mRNA. Processing of the premature RNA, for example, but not limited to, in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, a 5′ UTR, an open reading frame (ORF), a 3′ UTR, a poly(A) sequence, and/or any combination thereof.

In addition to messenger RNA, several non-coding types of RNA exist which may be involved in regulation of transcription and/or translation, and immunostimulation. The term “RNA” further encompasses RNA molecules, such as viral RNA, retroviral RNA and replicon RNA, small interfering RNA (siRNA), antisense RNA, CRISPR/Cas9 guide RNA, ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), and Piwi-interacting RNA (piRNA).

### 1. Ribonucleoside Triphosphates

The ribonucleoside triphosphates (NTPs) GTP, ATP, CTP and UTP are the monomers which are polymerized during the RNA polynucleotide synthesis process. They may be provided with a monovalent or divalent cation as counterion. In certain embodiments, the monovalent cation is selected from the group consisting of Li⁺, Na⁻, K⁻, NH⁴⁺ or tris(hydroxymethyl)-aminomethane (Tris). In certain embodiments, the divalent cation is selected from the group consisting of Mg²⁺, Ba²⁺ and Mn²⁺. In certain embodiments, the monovalent cation is Na⁺ or tris(hydroxymethyl)-aminomethane (Tris).

In certain embodiments, a part of, or all of, at least one ribonucleoside triphosphate in the RNA synthesis is replaced with a modified nucleoside triphosphate as defined below.

### 2. Modified Nucleoside Triphosphate

The term “modified nucleoside triphosphate” as used herein refers to chemical modifications comprising backbone modifications as well as sugar modifications or base modifications. These modified nucleoside triphosphates are also termed herein as “nucleotide analogs.”

In this context, the modified nucleoside triphosphates as defined herein are nucleotide analogs/modifications, for example, but not limited to, backbone modifications, sugar modifications or base modifications. A backbone modification in connection with the present disclosure is a modification, in which phosphates of the backbone of the nucleotides are chemically modified. A sugar modification in connection with the present disclosure is a chemical modification of the sugar of the nucleotides. Furthermore, a base modification in connection with the present disclosure is a chemical modification of the base moiety of the nucleotides. In this context nucleotide analogs or modifications are preferably selected from nucleotide analogs which are applicable for RNA synthesis and/or translation.

### 3. Sugar Modifications

The modified nucleosides and nucleotides, which may be used in the context of the present disclosure, can be modified in the sugar moiety. For example, but not limited to, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, for example, but not limited to, R=H, alkyl, cycloalkyl, aryl, aralkyl, heteroaryl or sugar); polyethyleneglycols (PEG), —O(CH2CH20)nCH2CH20R; “locked” nucleic acids (LNA) in which the 2′ hydroxyl is connected, for example, but not limited to, by a methylene bridge, to the 4′ carbon of the same ribose sugar; and amino groups (—O-amino, wherein the amino group, for example, but not limited to, NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (for example, but not limited to, NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and/or O.

The sugar group can also contain one or more carbons that possess the opposite stereochemical configuration than that of the corresponding carbon in ribose. Thus, a modified nucleotide can include nucleotides containing, for example, but not limited to, arabinose as the sugar.

### 4. Backbone Modifications

The phosphate backbone may further be modified in the modified nucleosides and nucleotides. The phosphate groups of the backbone can be modified by replacing one or more of the oxygen atoms with a different substituent. Further, the modified nucleosides and nucleotides can include the full replacement of an unmodified phosphate moiety with a modified phosphate. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. In certain embodiments, the phosphate linker can also be modified by the replacement of a linking oxygen with nitrogen (bridged phosphoroamidates), sulfur (bridged phosphorothioates) and carbon (bridged methylene-phosphonates).

### 5. Base Modifications

The modified nucleosides and nucleotides, which in certain embodiments, may be used in embodiments of the present disclosure, can further be modified in the nucleobase moiety. Examples of nucleobases found in RNA include, but are not limited to, adenine, guanine, cytosine and uracil. For example, the nucleosides and nucleotides described herein can be chemically modified on the major groove face. In certain embodiments, the major groove chemical modifications can include an amino group, a thiol group, an alkyl group, or a halo group.

In certain embodiments of the present disclosure, the nucleotide analogs/modifications are selected from base modifications including, but not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidinetriphosphate, 2-thiocytidine-5′-triphosphate, 2-thiouridine-5′-triphosphate, 2′-Fluorothymidine-5′-triphosphate, 2′-O-Methyl inosine-5′-triphosphate, 4-thiouridine-5′-triphosphate, 5-aminoally lcytidine-5′-triphosphate, 5-aminoally luridine-5′-triphosphate, 5-bromocytidine-5′triphosphate, 5-bromouridine-5′-triphosphate, 5-Bromo-2′deoxycytidine-5′-triphosphate, 5-Bromo-2′-deoxyuridine-5′triphosphate, 5-iodocytidine-5′-triphosphate, 5-Iodo-2′deoxycytidine-5′-triphosphate, 5-iodouridine-5′triphosphate, 5-Iodo-2′-deoxyuridine-5′-triphosphate, 5-methylcytidine-5′-triphosphate, 5-methyluridine-5′triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, puromycin-5′-triphosphate, xanthosine-5′-triphosphate, and/or any combinations thereof. In certain embodiments, nucleotides for base modifications comprise 5-methylcytidine-5′-triphosphate, 7-deazaguanosine-5′-triphosphate, 5-bromocytidine-5′-triphosphate, pseudouridine-5′-triphosphate, and/or any combinations thereof.

In certain embodiments, modified nucleosides comprise pyridin-4-one ribonucleoside, 5-aza-uridine, 2-thio-5-aza-uridine, 2-thiouridine, 4-thio-pseudouridine, 2-thiopseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methylpseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thiodihydrouridine, 2-thio-dihydropseudouridine, 2-methoxyuridine, 2-methoxy-4-thio-uridine, 4-methoxypseudouridine, 4-methoxy-2-thio-pseudouridine, and/or any combinations thereof.

In certain embodiments, modified nucleosides comprise 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thiopseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methylpseudoisocytidine, and/or any combinations thereof.

In certain embodiments, modified nucleosides comprise 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cishydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthioadenine, 2-methoxy-adenine, and/or any combinations thereof.

In certain embodiments, modified nucleosides comprise inosine, 1-methyl-inosine, wyosine, wybutosine, 7-deaza-guanosine, 7-deaza-8-aza-guanosine, 6-thioguanosine, 6-thio-7-deaza-guanosine, 6-thio-7-deaza-8-azaguanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, and/or any combinations thereof.

In certain embodiments, the nucleotide can be modified on the major groove face and can include replacing hydrogen on C-5 of uracil with a methyl group or a halo group.

In certain embodiments, a modified nucleoside comprises 5′-O-(1-Thiophosphate)-Adenosine, 5′-O-(1-Thiophosphate)-Cytidine, 5′-O-(1-Thiophosphate)-Guano sine, 5′-0(1-Thiophosphate)-Uridine, 5′-O-(1-Thiophosphate) Pseudouridinem, and/or any combinations thereof.

In certain embodiments the modified nucleotides comprise nucleoside modifications, for example, but not limited to, 6-aza-cytidine, 2-thio-cytidine, α-thio-cytidine, Pseudo-isocytidine, 5-aminoallyl-uridine, 5-iodo-uridine, N1-methylpseudouridine, 5,6-dihydrouridine, α-thio-uridine, 4-thiouridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-isocytidine, 6-Chloro-purine, N6-methyl-adenosine, α-thioadenosine, 8-azido-adenosine, 7-deaza-adenosine.

Further modified nucleotides have been described previously in WO 2013/052523 (the contents of which are incorporated herein by reference in their entirety).

### 6. Circular Polynucleotide Architecture

In certain embodiments, the present disclosure contemplates polynucleotides which are circular or cyclic. As the name implies, circular polynucleotides are circular in nature, meaning that the termini are joined in some fashion, whether by ligation, covalent bond, common association with the same protein or other molecule or complex or by hybridization.

In certain embodiments, circular polynucleotides (circPs) of the present disclosure which encode at least one peptide or polypeptide of interest are known as circular RNAs (circRNA). In certain embodiments, AAV and/or helper gene products of the present disclosure may be encoded by one or more circRNAs.

### 7. 5′-CAP

A 5′ cap is typically a modified nucleotide, particularly a guanine nucleotide, added to the 5′ end of an RNA molecule. In some embodiments, the 5′ cap is added using a 5′-5′-triphosphate linkage. In some embodiments, a 5′ cap may be methylated, for example, but not limited to, m7GpppN, wherein N is the terminal 5′ nucleotide of the nucleic acid carrying the 5′ cap, typically the 5′-end of an RNA. A naturally occurring 5′ cap is m7GpppN.

Further examples of 5′ cap structures include, but are not limited to, glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

In certain embodiments, 5′ cap structures are CAP1 (methylation of the ribose of the adjacent nucleotide of m7G), CAP2 (methylation of the ribose of the 2^{ad} nucleotide downstream of the m7G), CAP3 (methylation of the ribose of the 3^{rd} nucleotide downstream of the m7G) and CAP4 (methylation of the ribose of the 4^{th} nucleotide downstream of the m7G).

In certain embodiments, a 5′ cap structure may be formed by a cap analog. A 5′ cap analog refers to a non-extendable di-nucleotide that has cap functionality which means that it facilitates translation or localization, and/or prevents degradation of the RNA molecule when incorporated at the 5′ end of the RNA molecule. Non-extendable means that the cap analog will be incorporated only at the 5′ terminus because it does not have a 5′ triphosphate and therefore cannot be extended in the 3′ direction by a template dependent RNA polymerase.

5′ cap analogs include, but are not limited to, m7GpppG, m7GpppA, m7GpppC; unmethylated cap analogs (for example, but not limited to, GpppG); dimethylated cap analog (for example, but not limited to, m2,7GpppG), trimethylated cap analog (for example, but not limited to, m2,2,7GpppG), demethylated symmetrical cap analogs (for example, but not limited to, m7Gpppm7G), or anti-reverse cap analogs (for example, but not limited to, ARCA; m7,2′OmeGpppG, m7,2′dGpppG, m7,3′OmeGpppG, m7,3′dGpppG and their tetraphosphate derivatives) (Stepinski et al., 2001. RNA 7(10): 1486-95).

Further 5′ cap analogs have been described previously in U.S. Pat. No. 7,074,596, WO 2008/016473, WO 2008/157688, WO 2009/149253, WO 2011/015347, WO 2013/059475, and US 2019/0010485 A1 (all of which are incorporated herein by reference in their entirety for the purposes described herein). The synthesis of N7-(4-chlorophenoxyethyl) substituted dinucleotide cap analogs has been described in Kore et al., 2013 (see e.g., Bioorg. Med. Chem. 21(15):4570-4), the contents of which are incorporated herein by reference in their entirety for the purposes described herein.

In certain embodiments, 5′ cap analogs comprise G[5′]ppp[5′]G, m⁷G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{7,2′-O}GppspG D1 (β-S-ARCA D1), m₂^{7,2′-O}GppspG D2 (β-S-ARCA D2), and/or any combinations thereof.

In certain embodiments, a 5′ cap analog is added with an initial concentration in the range of about 1 to 20 mM, 1 to 17.5 mM, 1 to 15 mM, 1 to 12.5 mM, 1 to 10 mM, 1 to 7.5 mM, 1 to 5 mM or 1 to 2.5 mM. In certain embodiments, a 5′ cap analog is added with an initial concentration of about 5 to 20 mM, 7.5 to 20 mM, 10 to 20 mM or 12.5 to 20 mM.

### E. In Vitro Transcription

In the art, straight-forward processes for the recombinant production of RNA molecules in preparative amounts have been developed in a process called “RNA in vitro transcription”. The term “RNA in vitro transcription” relates to a process wherein RNA is synthesized in a cell-free system (in vitro). RNA is commonly obtained by enzymatic DNA dependent in vitro transcription of an appropriate DNA template, which is often a linearized plasmid DNA template. The promoter for controlling RNA in vitro transcription can be any promoter for any DNA dependent RNA polymerase. Particular examples of DNA dependent RNA polymerases are the bacteriophage enzymes T7, T3, and/or SP6 RNA polymerases.

Methods for RNA in vitro transcription are known in the art (see for example Geall et al. (2013) Semin. Immunol. 25(2): 152-159; Brunelle et al. (2013) Methods Enzymol. 530: 101-14). Reagents used in said methods may include: a linear DNA template with a promoter sequence that has a high binding affinity for its respective RNA polymerase; ribonucleoside triphosphates (NTPs) for the four bases (adenine, cytosine, guanine and uracil); a cap analog (for example, but not limited to, m7G(5′)ppp(5′)G (m7G)); other modified nucleotides; DNA-dependent RNA polymerase (for example, but not limited to, T7, T3 or SP6 RNA polymerase); ribonuclease (RNase) inhibitor to inactivate any contaminating RNasc; pyrophosphatase to degrade pyrophosphate, which inhibits transcription; MgCl₂, which supplies Mg²⁺ as a cofactor for the RNA polymerase; antioxidants (for example, but not limited to, DTT); polyamines such as spermidine; and a buffer to maintain a suitable pH value.

Common buffer systems used in RNA in vitro transcription include 4-(2-hydroxy-ethyl)-1-piperazineethanesulfonic acid (HEPES) and tris(hydroxymethyl) amino-methane (Tris). The pH value of the buffer is commonly adjusted to a pH value of about 6 to 8.5. Some commonly used transcription buffers comprise 80 mM HEPES/KOH, pH 7.5 and 40 mM Tris/HCl, pH 7.5.

The transcription buffer can also contain a magnesium salt such as MgCl₂ commonly in a range between 5-50 mM. Magnesium ions (Mg²⁺) are an essential component in an RNA in vitro transcription buffer system because free Mg²⁺ acts as cofactor in the catalytic center of the RNA polymerase and is critical for the RNA polymerization reaction. In diffuse binding, fully hydrated Mg²⁺, ions also interact with the RNA product via nonspecific long-range electrostatic interactions.

RNA in vitro transcription reactions are typically performed as batch reactions in which all components are combined and then incubated to allow the synthesis of RNA molecules until the reaction terminates. In addition, fed-batch reactions were developed to increase the efficiency of the RNA in vitro transcription reaction (see e.g., Kern et al. (1997) Biotechnol. Prag. 13: 747-756; and Kern et al. (1999) Biotechnol. Prog. 15: 174-184). In a fed-batch system, all components are combined, but then additional amounts of some of the reagents are added over time (for example, but not limited to, NTPs, and/or MgCl₂) to maintain constant reaction conditions.

Moreover, the use of a bioreactor (transcription reactor) for the synthesis of RNA molecules by in vitro transcription has been reported (see e.g., WO 1995/08626). The bioreactor is configured such that reactants are delivered via a feed line to the reactor core and RNA products are removed by passing through an ultrafiltration membrane (having a nominal molecular weight cut-off, for example, but not limited to, 100,000 Daltons) to the exit stream.

The concentration of the nucleic acid template comprised in the in vitro transcription mixture is in a range from about 1 to 50 nM, 1 to 40 nM, 1 to 30 nM, 1 to 20 nM, or about 1 to 10 nM. In certain embodiments, the concentration of the nucleic acid template is from about 10 to 30 nM. In certain embodiments, the concentration of the nucleic acid template is about 20 nM. In certain embodiments, a concentration of the nucleic acid template is of about 1 to 200 μg/ml, about 10 to 100 μg/ml, or about 20 to 50 μg/ml.

### 1. RNA Polymerase

The RNA polymerase is an enzyme which catalyzes the transcription of a DNA template into RNA. In certain embodiments, suitable RNA polymerases for use in methods and/or compositions of the present disclosure include, but are not limited to, T7, T3, SP6 and E. coli RNA polymerase. In certain embodiments, a T7 RNA polymerase is used. In certain embodiments, an RNA polymerase for use in the present disclosure is a recombinant RNA polymerase, meaning that it is added to the RNA in vitro transcription reaction as a single component and not as part of a cell extract which contains other components in addition to the RNA polymerase. The skilled person knows that the choice of the RNA polymerase depends on the promoter present in the DNA template which has to be bound by the suitable RNA polymerase. In certain embodiments, the concentration of the RNA polymerase is from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the RNA polymerase is from about 10 to 50 nM, 20 to 50 nM, or 30 to 50 nM. In certain embodiments, the RNA polymerase concentration is about 40 nM. In certain embodiments, a concentration of 500 to 10000 U/ml of the RNA polymerase is used. In certain embodiments, a concentration of 1000 to 7500 U/ml, or a concentration of 2500 to 5000 Units/ml of the RNA polymerase is used. The person skilled in the art will understand that the choice of the RNA polymerase concentration is influenced by the concentration of the DNA template.

### 2. Pyrophosphatase

A pyrophosphatase is an acid anhydride hydrolase that hydrolyses diphosphate bonds. In an in vitro transcription reaction it serves to hydrolyze the bonds within the diphosphate released upon incorporation of the ribonucleoside triphosphates into the nascent RNA chain. In certain embodiments, the concentration of the pyrophosphatase is from about 1 to 20 units/ml, 1 to 15 units/ml, 1 to 10 units/ml, 1 to 5 units/ml, or 1 to 2.5 units/ml. In certain embodiments, the concentration of the pyrophosphatase is about 1 unit/ml or is about 5 units/ml.

### 3. Ribonuclease Inhibitor

A ribonuclease inhibitor inhibits the action of a ribonuclease which degrades RNA. In certain embodiments, the concentration of the ribonuclease inhibitor is from about 1 to 500 units/ml, 1 to 400 units/ml, 1 to 300 units/ml, 1 to 200 units/ml, or 1 to 100 units/ml. In certain embodiments, the concentration of the ribonuclease inhibitor is about 200 units/ml.

### 4. Antioxidant

An antioxidant inhibits the oxidation of other molecules. Suitable antioxidants for use in the present disclosure include, but are not limited to, DTT (dithiothreitol), TCEP (tris(2-carboxyethyl)phosphine), NAC (N-acetylcysteine), beta-mercaptoethanol, glutathione, cysteine and cystine. In certain embodiments, DTT is used in the in vitro transcription reaction.

In certain embodiments, the concentration of DTT is about 1 to 50 mM, 5 to 48 mM, 8 to 47 mM, 10 to 46 mM, 15 to 45 mM, 18 to 44 mM, 20 to 43 mM, 23 to 42 mM, 25 to 41 mM or 28 to 40 mM. In certain embodiments, the concentration of DTT is 40 mM.

### F. Solid-Phase Chemical Synthesis

Polynucleotides or circular polynucleotides of the present disclosure may be manufactured in whole or in part using solid phase techniques.

Solid-phase chemical synthesis of polynucleotides or nucleic acids is an automated method wherein molecules are immobilized on a solid support and synthesized step by step in a reactant solution. Impurities and excess reagents are washed away and no purification is required after each step. The automation of the process is amenable on a computer controlled solid-phase synthesizer. Solid-phase synthesis allows rapid production of polynucleotides or nucleic acids in a relatively large scale that leads to the commercial availability of some polynucleotides or nucleic acids. Furthermore, it is useful in site-specific introduction of chemical modifications in the polynucleotide or nucleic acid sequences. It is an indispensable tool in designing modified derivatives of natural nucleic acids.

In automated solid-phase synthesis, the chain is synthesized in 3′ to 5′ direction. The hydroxyl group in the 3′ end of a nucleoside is tethered to a solid support via a chemically cleavable or light-cleavable linker. Activated nucleoside monomers, such as 2′-deoxynucleosides (dA, dC, dG, and T), ribonucleosides (A, C, G, and U), or chemically modified nucleosides, are added to the support-bound nucleoside sequentially. Currently, most widely utilized monomers are the 3′-phosphoramidite derivatives of nucleoside building blocks. The 3′ phosphorus atom of the activated monomer couples with the 5′ oxygen atom of the support-bound nucleoside to form a phosphite triester. To prevent side reactions, all functional groups not involved in the coupling reaction, such as the 5′ hydroxyl group, the hydroxyl group on the 3′ phosphorus atom, the 2′ hydroxyl group in ribonucleosides monomers, and the amino groups on the purine or pyrimidine bases, may all be blocked with protection groups. The next step may involve oxidation of the phosphite triester to form a phosphate triester or phosphotriester, where the phosphorus atom is pentavalent. The protection group on the 5′ hydroxyl group at the end of the growing chain may then be removed, ready to couple with an incoming activated monomer building block. At the end of the synthesis, a cleaving agent such as ammonia or ammonium hydroxide may be added to remove all the protecting groups and release the polynucleotide chains from the solid support. Light may also be applied to cleave the polynucleotide chain. The product can then be further purified with high pressure liquid chromatography (HPLC) or electrophoresis.

In solid-phase synthesis, the polynucleotide chain may be covalently bound to the solid support via its 3′ hydroxyl group. The solid supports are insoluble particles also called resins, typically 50-200 m in diameter. Many different kinds of resins are now available, as reviewed in “Solidphase supports for polynucleotide synthesis” by Guzaev (see e.g., Guzaev, Current Protocols in Nucleic Acid Chemistry, 3.1.1-3.1.60 (2013), the contents of which are incorporated herein by reference in their entirety for the purposes described herein). The most common materials for the resins include highly cross-linked polystyrene beads and controlled pore glass (CPG) beads. The surface of the beads may be treated to have functional groups, such as amino or aminomethyl groups that can be used as anchoring points for linkers to tether nucleosides. They can be implemented in columns, multi-well plates, microarrays or microchips. The column-based format allows relatively large scale synthesis of the polynucleotides or nucleic acids. The resins are held between filters in columns that enable all reagents and solvents to pass through freely. Multi-well plates, microarrays, or microchips are designed specifically for cost-effective small scale synthesis. Up to a million polynucleotides can be produced on a single microarray chip. However, the error rates of microchip based synthesis are higher than traditional column-based methods (Borovkov et al., Nucleic Acids Research, vol. 38(19), e180 (2010)), the contents of which are incorporated herein by reference in their entirety for the purposes described herein). Multi-well plates allow parallel synthesis of polynucleotides or nucleic acids with different sequences simultaneously (Sindelar, et al., Nucleic Acids Research, vol. 23, 982-987 (1995), the contents of which are incorporated herein by reference in their entirety for the purposes described herein). The loading on the solid supports is limited. In addition, as the extension progresses, the morphology and bulkiness of the growing chains on the solid supports might hinder the incoming monomers from reacting with the terminal group of the growing chains. Therefore, the number of monomers that can be added to the growing chain is also limited.

Linkers may be attached to the solid support for further extension of the chain. They are stable to all the reagents used in the synthesis process, except in the end of the synthesis when the chain is detached from the solid support. Solid supports with a specific nucleoside linker, for example, but not limited to, A, C, dT, G, or U, can be used to prepare polynucleotides with A, C, T, G, or U as the first nucleotide in the sequence, respectively. Universal solid supports with non-nucleoside linkers can be used for all polynucleotide sequences (U.S. Pat. No. 6,653,468 to Guzaev et al., the contents of which are incorporated herein by reference in their entirety for the purposes described herein). Various non-nucleoside linkers have been developed for universal supports, a lot of them with two vicinal hydroxyl groups. For example, but not limited to, a succinyl group is a frequently used linker.

As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety. A linker may be nucleic acid based or non-nucleosidic. The linker may be of sufficient length as to not interfere with incorporation into a nucleic acid sequence. The linker can be used for any useful purpose, such as to form multimers (for example, but not limited to, through linkage of two or more chimeric polynucleotides molecules) or conjugates, as well as to administer a therapeutic molecule or incorporate a label, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (for example, but not limited to, ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, but not limited to, a disulfide bond (—S—S—) or an azo bond (—N N—), which can be cleaved using a reducing agent or photolysis. Non-limiting examples of a selectively cleavable bond include an amido bond that can be cleaved for example, but not limited to, by the use of tris(2-carboxyethyl)phosphine (TCEP), or other reducing agents, and/or photolysis, as well as an ester bond that can be cleaved for example, but not limited to, by acidic or basic hydrolysis.

Besides the functional groups on the activated monomer and the growing chain needed for the coupling reaction to extend the chain, all other functional groups need to be protected to avoid side reactions. The conditions for protection and deprotection, and the selection of appropriate protecting groups can be readily determined by one skilled in the art. The chemistry of protecting groups can be found and/or described, for example, but not limited to, in Greene, et al. “Protective Groups in Organic Synthesis”, 2d. Ed., Wiley & Sons, 1991 (the contents of which is incorporated herein by reference in its entirety for the purposes described herein). For example, the 5′ hydroxyl group on the activated nucleoside phosphoramidite monomers may be protected with 4,4′-dimethoxytrityl (DMT) and the hydroxyl group on the phosphorus atom may be protected with 2-cyanoethyl. The exocyclic amino groups on the A, C, G bases may be protected with acyl groups.

In a solid-phase synthesis system, the reactivity of the activated monomers is important, because of the heterogeneity of the media. A majority of solid-phase synthesis uses phosphoramidite nucleosides, the mechanism of which is discussed above. Another activated monomer example is nucleoside H-phosphonates (see e.g., Abramova, Molecules, vol. 18, 1063-1075 (2013), the contents of which are incorporated herein by reference in their entirety for the purposes described herein). A large excess of reagents, such as monomers, oxidizing agents, and deprotection agents, may be required in order to ensure high yields in the solid-phase synthesis system.

Scientific studies and research are going on to further improve the solid-phase synthesis method. For example, instead of the well-established 3′-to-5′ synthesis, U.S. Pat. No. 8,309,707 and US Pat. Publication No. 2013/0072670, the contents of which are incorporated herein by reference in their entirety for the purposes described herein, disclosed a 5′-to-3′ synthesis of RNA utilizing a novel phosphoramidite and a novel nucleoside derivative, thereby allowing easy modifications of the synthetic RNA at the 3′ end. PCT application PCT/US2013/026045 published as WO2013123125A1, the contents of which are incorporated herein by reference in their entirety for the purposes described herein, describes assembly of a target nucleic acid sequence from a plurality of subsequences, wherein resins with the subsequences are placed in an emulsion droplet. The subsequences are cleaved off the resins and assemble within the emulsion droplet. To reduce the cost of solid supports, a reusable CPG solid support has been developed with a hydroquinone-O, O′-diacetic acid linker (Q-linker) (Pon et al., Nucleic Acid Research, vol. 27, 1531-1538 (1999), the contents of which are incorporated herein by reference in their entirety for the purposes described herein).

New protecting groups for solid-phase synthesis have also been developed. Nagata et al. 2010 (see e.g., Nucleic Acid Res. 38(21):7845-57) has successfully synthesized 110-nt-long RNA with the sequence of a candidate precursor microRNA by using 2-cyanoethoxymethyl (CEM) as the 2′-hydroxy protecting group (Shiba et al., Nucleic Acids Research, vol. 35, 3287-3296 (2007), the contents of which are incorporated herein by reference in their entirety). Also with CEM as 2-O-protecting group, a 130-nt mRNA has been synthesized encoding a 33-amino acid peptide that includes the sequence of glucagon-like peptide-I (GLP-1). The biological activity of the artificial 130-nt mRNA is shown by producing GLP-1 in a cell-free protein synthesis system and in Chinese hamster ovary (CHO) cells (Nagata et al., Nucleic Acids Research, vol. 38(21), 7845-7857 (2010), the contents of which are incorporated herein by reference in their entirety for the purposes described herein). Protecting groups for solid-phase synthesis monomers include, but are not limited to, carbonate protecting groups disclosed in U.S. Pat. No. 8,309,706, orthoester-type 2′-hydroxyl protecting group and an acyl carbonate-type hydroxyl protecting group disclosed in U.S. Pat. No. 8,242,258, 2′-hydroxyl thiocarbon protecting group disclosed in U.S. Pat. No. 8,202,983, 2′-silyl containing thiocarbonate protecting group disclosed in U.S. Pat. No. 7,999,087, 9-fluorenylmethoxycarbonyl (FMOS) derivatives as an amino protecting group disclosed in U.S. Pat. No. 7,667,033, fluoride-labile 5′-silyl protecting group disclosed in U.S. Pat. No. 5,889,136, and pixyl protecting groups disclosed in US Pat. Publication No. 2008/0119645, US Pat. Publication No. 2011/0275793 teaches RNA synthesis using a protecting group of the hyoxyls in position 2 of the ribose that can be removed by a base, solid supports include polymers made from monomers comprising protected hydroxypolyC2-4 alkyleneoxy chain attached to a polymerizable unit taught in U.S. Pat. No. 7,476,709, the contents of each of the aforementioned are incorporated herein by reference in their entirety for the purposes described herein.

### G. Liquid Phase Chemical Synthesis

In certain embodiments, synthesis of recombinant polynucleotides or circular polynucleotides of the present disclosure by the sequential addition of monomer building blocks may be carried out in a liquid phase. A covalent bond is formed between the monomers or between a terminal functional group of the growing chain and an incoming monomer. Functional groups not involved in the reaction must be temporarily protected. After the addition of each monomer building block, the reaction mixture may be purified before adding the next monomer building block. The functional group at one terminal of the chain may be deprotected to be able to react with the next monomer building blocks. A liquid phase synthesis is labor- and time-consuming and may not be automated. Despite the limitations, liquid phase synthesis is still useful in preparing short polynucleotides in a large scale. Because the system is homogenous, it does not require a large excess of reagents and is cost effective in this respect.

### H. Nucleotide Purification

Purification of the polynucleotides of the disclosure described herein may include, but is not limited to, polynucleotide clean-up, quality assurance and quality control. Clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), hydrophobic interaction HPLC (HIC-HPLC), and/or any combinations thereof. The term “purified” when used in relation to a polynucleotide such as a “purified polynucleotide” refers to one that is separated from at least one contaminant. As used herein, a “contaminant” is any substance which makes another unfit, impure, and/or inferior. Thus, a purified polynucleotide (for example, but not limited to, DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In certain embodiments, the polynucleotides may be sequenced by methods including, but not limited, to reverse-transcriptase-PCR.

### I. Transfection

As used herein, the term “transfection” is defined as the introduction of an extracellular nucleic acid into a host cell by any means known in the art, including, but not limited to, calcium phosphate co-precipitation, viral transduction, liposome fusion, microinjection, microparticle bombardment, electroporation. The terms “uptake of nucleic acid by a host cell”, “taking up of nucleic acid by a host cell”, “uptake of particles comprising nucleic acid by a host cell”, and “taking up of particles comprising nucleic acid by a host cell” denote any process wherein an extracellular nucleic acid, with or without accompanying material, enters a host cell.

A variety of methods are known in the art and suitable for transfection of nucleic acid into a cell. The polynucleotides of the present disclosure may be formulated, using the methods described herein. The formulations may comprise polynucleotides which may be modified and/or unmodified. The formulations may further comprise, but are not limited to, cell penetration agents, a pharmaceutically acceptable carrier, a delivery agent, a bioerodible or biocompatible polymer, a solvent, a sustained-release delivery depot, and/or any combinations thereof.

The formulated polynucleotides may be delivered to the cell using routes of administration known in the art and described herein. Examples of typical methods include, but are not limited to, naked delivery, lipidoid mediate transfer, liposome-, lipoplexes, and/or lipid nanoparticle-mediated transfer, electroporation, calcium phosphate mediated transfer, nucleofection, sonoporation, heat shock, magnetofection, microinjection, microprojectile mediated transfer (for example, but not limited to, nanoparticles), cationic polymer mediated transfer (for example, but not limited to, DEAE-dextran, polyethylenimine, polyethylene glycol (PEG) and the like) or cell fusion.

### 1. Naked Delivery

The polynucleotides of the present disclosure may be delivered to a cell naked. As used herein in, “naked” refers to delivering polynucleotides free from agents which promote transfection. For example, but not limited to, the polynucleotides delivered to the cell may contain no modifications and/or delivery agents. The naked polynucleotides may be delivered to the cell using routes of administration known in the art and described herein. In certain embodiments, the polynucleotides are delivered naked and the cells uptake them by endocytosis.

### 2. Lipidoids

The synthesis of lipidoids has been extensively described and formulations containing these compounds are particularly suited for delivery of polynucleotides (see e.g., Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107: 1864-1869; and Siegwart et al., Proc Natl Acad Sci USA. 2011 108:12996-3001; all of which are incorporated herein by reference in their entireties for the purposes described herein).

While these lipidoids have been used to effectively deliver double stranded small interfering RNA molecules in rodents and non-human primates (see e.g., Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010; all of which are incorporated herein by reference in their entirety for the purposes described herein), lipidoids may be used with the formulations of the present disclosure for delivering one or more of the polynucleotides contained herein.

Complexes, micelles, liposomes or particles may be prepared containing these lipidoids and therefore, can result in an effective delivery of the polynucleotide, as judged by the production of an encoded protein (for example, but not limited to, AAV proteins), following the delivery of a lipidoid formulation to one or more host cells.

Delivery of nucleic acids may be affected by many parameters, including, but not limited to, the formulation composition, nature of particle PEGylation, degree of loading, polynucleotide to lipid ratio, and biophysical parameters such as, but not limited to, particle size (see e.g., Akinc et al., Mol Ther. 2009 17:872-879; herein incorporated by reference in its entirety for the purposes described herein). As an non-limiting example, small changes in the anchor chain length of poly(ethylene glycol) (PEG) lipids may result in significant effects on transfection efficacy. Formulations with the different lipidoids, including, but not limited to penta[3-(1-laury laminopropionyl)]-triethylenetetramine hydrochloride (TETA-5LAP; aka 98N12-5, see Murugaiah et al., Analytical Biochemistry, 401:6-7 (2010); herein incorporated by reference in its entirety for the purposes described herein), C12-200 (including derivatives and variants), MDI, and/or any combinations thereof.

The lipidoid formulations can include particles comprising either 3 or 4 or more components in addition to polynucleotides. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In certain embodiment, the polynucleotides may be formulated in an aminoalcohol lipidoid. Aminoalcohol lipidoids which may be prepared by the methods described in U.S. Pat. No. 8,450,298, herein incorporated by reference in its entirety for the purposes described herein.

### 3. Liposomes, Lipoplexes, and Lipid Nanoparticles

In certain embodiments, compositions and/or formulations of the present disclosure can be formulated using one or more liposomes, lipoplexes, lipid nanoparticles, and/or any combinations thereof. In certain embodiments, delivery compositions comprise liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUY) which may be between 50 and 500 nm in diameter. Liposome design may include, but is not limited to, opsonins or ligands in order to improve the attachment of liposomes to cells or to activate events such as, but not limited to, endocytosis. Liposomes may contain a low or a high pH to improve the delivery of the formulations.

The formation of liposomes may depend on the physicochemical characteristics such as, but not limited to, the polynucleotide formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in their entirety for the purposes described herein.

In certain embodiments, compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), MC3 (US20100324120; herein incorporated by reference in its entirety for the purposes described herein), liposomes which may deliver small molecule drugs such as, but not limited to, DOXIL® from Janssen Biotech, Inc. (Horsham, Pa.), and/or any combinations thereof.

In certain embodiments, compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and/or in vivo (see e.g., Wheeler et al. Gene Therapy. 1999 6:271-281; Zhang et al. Gene Therapy. 1999 6:1438-1447; Jeffs et al. Phann Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; each of which are incorporated herein by reference in their entireties for the purposes described herein). The original manufacture method by Wheeler et al. was a detergent dialysis method, which was later improved by Jeffs et al. and is referred to as the spontaneous vesicle formation method. In certain embodiments, liposome formulations are composed of 3 to 4 lipid components in addition to the polynucleotide. A liposome can contain, for example, but not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In certain embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In certain embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In certain embodiments, the polynucleotides may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety for the purposes described herein.

In certain embodiments, the lipid formulation may include at least a cationic lipid, a lipid which may enhance transfection and/or a least one lipid which contains a hydrophilic head group linked to a lipid moiety (see e.g., International Pub. No. WO2011076807 and U.S. Pub. No. 20110200582; the contents of each of which are herein incorporated by reference in their entirety for the purposes described herein). In certain embodiments, the polynucleotides may be formulated in a lipid vesicle which may have crosslinks between functionalized lipid bilayers (see e.g., U.S. Pub. No. 20120177724, the contents of which is herein incorporated by reference in its entirety for the purposes described herein).

In certain embodiments, the polynucleotides may be formulated in a liposome as described in International Patent Publication No. WO2013086526, the contents of which is herein incorporated by reference in its entirety for the purposes described herein. The polynucleotides may be encapsulated in a liposome using reverse pH gradients and/or optimized internal buffer compositions as described in International Patent Publication No. WO2013086526, the contents of which is herein incorporated by reference in its entirety for the purposes described herein.

In certain embodiments, the polynucleotide compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (Landen et al. Cancer Biology & Therapy 2006 5(12)1708-1713); herein incorporated by reference in its entirety) hyaluronan-coated liposomes (Quiet Therapeutics, Israel), and/or any combinations thereof.

In certain embodiments, the cationic lipid may be a low molecular weight cationic lipid such as those described in US Patent Application No. 20130090372, the contents of which are herein incorporated by reference in its entirety for the purposes described herein.

In certain embodiments, the polynucleotides may be formulated in a lipid vesicle which may have crosslinks between functionalized lipid bilayers.

In certain embodiments, the polynucleotides may be formulated in a liposome comprising a cationic lipid. The liposome may have a molar ratio of nitrogen atoms in the cationic lipid to the phosphates in the RNA (N:P ratio) of between 1:1 and 20:1 as described in International Publication No. WO2013006825, herein incorporated by reference in its entirety for the purposes described herein. In certain embodiments, the liposome may have a N:P ratio of greater than 20:1 or less than 1:1.

In certain embodiments, the polynucleotides may be formulated in a lipid-polycation complex. The formation of the lipidpolycation complex may be accomplished by methods known in the art and/or as described in U.S. Pub. No. 20120178702, herein incorporated by reference in its entirety for the purposes described herein. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety for the purposes described herein. In certain embodiments, the polynucleotides may be formulated in a lipid-polycation complex which may further include a non-cationic lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. As a non-limiting example by Semple et al. (see e.g., Semple et al. Nature Biotech. 2010 28:172-176; herein incorporated by reference in its entirety for the purposes described herein), the liposome formulation was composed of 57.1% cationic lipid, 7.1% dipalmitoylphosphatidylcholine (DPPC), 34.3% cholesterol, and 1.4% PEG-c-DMA. As a non-limiting example, changing the composition of the cationic lipid could more effectively deliver siRNA to various antigen presenting cells (see e.g., Basha et al. Mol Ther. 2011 19:2186-2200; herein incorporated by reference in its entirety for the purposes described herein). In certain embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1.

In certain embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-cDOMG (R-3-[(w-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In certain embodiments, the PEG-cDOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200, DLin-KC2-DMA, and/or any combinations thereof.

In certain embodiments, the LNP formulation may be formulated by the methods described in International Publication Nos. WO2011127255 or WO2008103276, the contents of each of which are herein incorporated by reference in their entirety for the purposes described herein. As a non-limiting example, modified RNA described herein may be encapsulated in LNP formulations as described in WO2011127255 and/or WO2008103276; the contents of each of which are herein incorporated by reference in their entirety for the purposes described herein.

### 4. Sonoporation

The technique of sonoporation, or cellular sonication, is the use of sound (for example, but not limited to, ultrasonic frequencies) for modifying the permeability of the cell plasma membrane. Sonoporation methods are known to those in the art and are used to deliver nucleic acids (see e.g., Yoon and Park, Expert Opin Drug Deliv. 2010 7:321-330; Postema and Gilja, Curr Pharm Biotechnol. 2007 8:355-361; and Newman and Bettinger, Gene Ther. 2007 14:465-475; each of which is herein incorporated by reference in their entirety for the purposes described herein). Sonoporation methods are known in the art and are also taught for example, but not limiting to, as it relates to bacteria in US Patent Publication 20100196983 and as it relates to other cell types in, for example, but not limited to, US Patent Publication 20100009424, each of which are incorporated herein by reference in their entirety for the purposes described herein.

### 5. Electroporation

Electroporation techniques are also well known in the art and are used to deliver nucleic acids to cells (see e.g., Andre et al., Curr Gene Ther. 2010 10:267-280; Chiarella et al., Curr Gene Ther. 2010 10:281-286; Hojman, Curr Gene Ther. 2010 10:128-138; each of which are herein incorporated by reference in their entirety for the purposes described herein). Electroporation devices are sold by many companies worldwide including, but not limited to, BTX® Instruments (Holliston, Mass.) (e.g., the AgilePulse In Vivo System) and Inovio (Blue Bell, Pa.) (e.g., Inovio SP-5P intramuscular delivery device or the CELLECTRA® 3000 intradermal delivery device).

### 6. Calcium Phosphate

In certain embodiments, transfection is mediated by calcium phosphate. In general, calcium phosphate transfection proceeds as follows. Nucleic acid associates strongly with the calcium phosphate particles formed in a calcium phosphate precipitate. In the presence of host cells, calcium phosphate particles carrying nucleic acid contact the host cell surface, and the nucleic acid enters the host cell, at least in part through endocytosis and/or phagocytosis.

As previously described (EP 0779931, the contents of which are herein incorporated in their entirety for the purposes described herein), there is a correlation between the host cell's ability to take up nucleic acid and the size of the nucleic acid-calcium phosphate particles, and there is an optimum particle size that maximizes the host cell's ability to uptake nucleic acids. The limitation of particle size can increase transfection efficiency by increasing the number of particles that come into contact with a host cell. The limitation of particle growth after particles reach a particular size reduces the decline in particle number caused by aggregation or rearrangement of smaller particles. The limitation of particle size can also increase transfection efficiency by optimizing the particle surface area available for association with nucleic acid. In certain embodiments, the optimum particle size may be any size up to about 300 nm in length, wherein “length” is defined as the diameter at the widest part of the particle as measured by laser light scattering according to the method of Weiss and Frock, “Rapid Analysis of Particle Size Distribution by Laser Light Scattering”, Powder Technology, 14: 287 (1976).

In certain embodiments, the calcium phosphate transfection method may further comprise treatment of cells with chloroquine and/or glycerol. Both chloroquine and glycerol are known to improve transfection (see e.g., Molecular Cloning, 3^{rd} edition, Sambrook and Russell, Cold Spring Harbor Laboratory Press, 2001).

### J. Viral Vectors

Among other things, the present disclosure provides that in certain embodiments, gene products as described herein are encoded by a polynucleotide, for example, but not limited to, a vector or RNA molecule. Vectors comprising polynucleotide constructs according to the present disclosure include all those known in the art, including cosmids, plasmids (e.g., naked or contained in liposomes) and viral constructs (e.g., lentiviral, retroviral, adenoviral, and adeno associated viral constructs) that incorporate a polynucleotide comprising a desired gene product or characteristic portion thereof (e.g., as utilized herein, a “characteristic portion thereof” refers to the portion of said protein required to perform the desired function. Those of skill in the art will be capable of selecting suitable constructs, as well as cells, for making any of the polynucleotides described herein. In certain embodiments, a construct is a plasmid (i.e., a circular DNA molecule that can autonomously replicate inside a cell). In certain embodiments, a construct can be a cosmid (e.g., pWE or sCos series). In certain embodiments, a construct can be one or more RNA molecules.

In certain embodiments, a construct is a viral construct. In certain embodiments, a viral construct is a lentivirus, retrovirus, adenovirus, or adeno-associated virus. In certain embodiments, a viral construct is an adenovirus construct. In certain embodiments, a construct is an adeno-associated virus (AAV) construct or a recombinant AAV (rAAV) (see, e.g., Asokan et al., Mol. Ther. 20: 699-7080, 2012, which is incorporated herein by reference for the purposes described herein). In certain embodiments, the AAV or rAAV may be, or be derived from, AAV-1, AAV-2, AAV-3, AAV-4, AAV-5, AAV-6, AAV-7, AAV-8, AAV-9, AAV-10, AAV-11, and/or AAV-12, AAV-DJ, AAV-DJ8 as well as bovine AAV and avian AAV, pseudotyped, and/or chimeric. In certain embodiments, a viral construct may also be based on, or derived from, an alphavirus. Alphaviruses include but are not limited to, Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference for the purposes described herein.

In certain embodiments, constructs provided herein can be of different sizes. In certain embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In certain embodiments, a construct is a viral construct and can have a total number of nucleotides of up to 10 kb. In certain embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In certain embodiments, a construct is a lentivirus construct and can have a total number of nucleotides of up to 8 kb. In certain embodiments, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In certain embodiments, a construct is an adenovirus construct and can have a total number of nucleotides of up to 8 kb. In certain embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In certain embodiments, a promoter can be a native promoter, a constitutive promoter, an inducible promoter, and/or a tissue-specific promoter. Non-limiting examples of control sequences are described herein.

### 1. AAV Particles

Among other things, the present disclosure provides AAV particles that comprise a polynucleotide construct encoding one or more gene products, for example, but not limited to a gene product selected for use in gene therapy, and an AAV capsid. In certain embodiments, AAV particles can be described as having a serotype, which is a description of the construct strain and the capsid strain. For example, but not limited to, in some embodiments an AAV particle may be described as AAV2, wherein the particle has an AAV2 capsid and a construct that comprises characteristic AAV2 Inverted Terminal Repeats (ITRs). In certain embodiments, an AAV particle may be described as a pseudotype, wherein the capsid and construct are derived from different AAV strains, for example, but not limited to, AAV2/9 would refer to an AAV particle that comprises a construct utilizing the AAV2 ITRs and an AAV9 capsid. Additional non-limiting examples of pseudotyped AAV vectors include, but are not limited to, AAV2/1, AAV2/2, AAV2/3, AAV2/4, AAV2/5, AAV2/6, AAV2/7, AAV2/8, and AAV2/9.

In certain embodiments, AAV particles suitable for use according to the present disclosure may comprise or be derived from any natural or recombinant AAV serotype. In certain embodiments, an AAV according to the present disclosure is selected from natural serotypes such as AAV1, AAV2, AAV3, AAV4, AAV5, AAV6, AAV7, AAV8, AAV9, AAV10, AAV11, and AAV12; or pseudotypes, chimeras, and variants thereof.

As used herein, the term “chimera” when referring to an AAV vector, or a “chimeric AAV vector”, refers to an AAV vector which comprises a capsid containing VP1, VP2 and VP3 proteins from at least two different AAV serotypes; or alternatively, which comprises VP1, VP2 and VP3 proteins, at least one of which comprises at least a portion from another AAV serotype. Examples of chimeric AAV vectors include, but are not limited to, AAV-DJ, AAV-DJ/8, AAV2G9, AAV2i8, AAV2i8G9, AAV8G9, and AAV9i1.

In certain embodiments, an AAV serotype and/or pseudotype according to the present disclosure is selected from the group comprising or consisting of AAV1, AAV2, AAV3, AAV 4, AAV5, AAV6, AAV7, AAV8, AAV9, AAV10, AAV11, AAV12, AAV106.1/hu.37, AAV114.3/hu.40, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2i8, AAV2i8G9, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV2.5T, AAV27.3, AAV29.3/bb.1, AAV29.5/bb.2, AAV2G9, AAV3B, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rh.64, AAV4-9/rh.54, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6.1, AAV6.1.2, AAV6.2, AAV7m8, AAV7.2, AAV7.3/hu.7, AAV-8b, AAV8G9, AAV-8h, AAV9i1, AAV9.11, AAV9.13, AAV9.16, AAV9.24, AAV9.45, AAV9.47, AAV9.61, AAV9.68, AAV9.84, AAV9.9, AAVcy.2, AAVcy.3, AAVcy.4, AAVcy.5, AAVcy.5R1, AAVcy.5R2, AAVcy.5R3, AAVcy.5R4, AAVcy.6, AAVhu.1, AAVhu.2, AAVhu.3, AAVhu.4, AAVhu.5, AAVhu.6, AAVhu.7, AAVhu.8, AAVhu.9, AAVhu.10, AAVhu.11, AAVhu.12, AAVhu.13, AAVhu.14/9, AAVhu.15, AAVhu.16, AAVhu.17, AAVhu.18, AAVhu.19, AAVhu.20, AAVhu.21, AAVhu.22, AAVhu.23.2, AAVhu.24, AAVhu.25, AVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.53, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVpi.1, AAVpi.2, AAVpi.3, AAVrh.2, AAVrh.2R, AAVrh.8, AAVrh.8R, AAVrh8R R533A mutant, AAVrh8R A586R mutant, AAVrh.10, AAVrh.12, AAVrh.13, AAVrh. 13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAV-PHP.B, AAVPHP.A, AAV-G2B-26, AAV-G2B-13, AAV-TH1.1-32, AAVTH1.1-35, AAV-PHP.B2, AAV-PHP.B3, AAV-PHP.N/PHP.B-DGT, AAV-PHP.B-EST, AAV-PHP.B-GGT, AAV-PHP.BATP, AAV-PHP.B-ATT-T, AAV-PHP.B-DGT-T, AAV-PHP.B-GGT-T, AAV-PHP.B-SGS, AAV-PHP.B-AQP, AAV-PHP.B-QQP, AAV-PHP.B-SNP(3), AAV-PHP.B-SNP, AAV-PHP.B-QGT, AAV-PHP.B-NQT, AAV-PHP.B-EGS, AAV-PHP.BSGN, AAV-PHP.B-EGT, AAV-PHP.B-DST, AAV-PHP.BDST, AAV-PHP.B-STP, AAV-PHP.B-PQP, AAV-PHP.BSQP, AAV-PHP.B-Q1P, AAV-PHP.B-TMP, AAV-PHP.BTTP, AAV-PHP.S/G2A12, AAV-G2A15/G2A3, AAV-G2B4, AAV-G2B5, PHP.S, AAAV, AAV A3.3, AAV A3.4, AAV A3.5, AAV A3.7, AAV CBr-7.3, AAV CBr-7.1, AAV CBr-7.10, AAV CBr-7.2, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-N4, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKdB4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-M9, AAV CLv-R6, AAV CLv-1, AAV CLvl-1, AAV CLyl-10, AAV CLvl-2, AAV CLv-12, AAV CLvl-3, AAV CLv-13, AAV CLvl-4, AAV CLvl-7, AAV CLvl-8, AAV CLvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLvM7, AAV CLv-M8, AAV CLv-R1, AAV CLv-R2, AAV CLv-R3, AAV CLv-R4, AAV CLv-R5, AAV CLv-R7, AAV CLv-R8, AAV CLv-R9, AAV CSp-8.10, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.2, AAV CSp-8.4, AAV CSp-8.5, AAV CSp-8.6, AAV CSp-8.7, AAV CSp-8.8, AAV CSp-8.9, AAV CSp-9, AAVLK08, AAV-LK15, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV.VR-355, AAV-b, AAVC1, AAVC2, AAVC5, AAVCh.5, AAVCh.5R1, AAV-DJ, AAV-DJ8, AAVF1/HSC1, AAVF1l/HSC11, AAVF12/HSC12, AAVF13/HSC13, AAVF14/HSC14, AVF15/HSC15, AAVF16/HSC16, AAVF17/HSC17, AAVF2/HSC2, AAVF3, AAVF3/HSC3, AAVF4/HSC4, AAVF5, AAVF5/HSC5, AAVF6/HSC6, AAVF7/HSC7, AAVF8/HSC8, AAVF9/HSC9, AAV-h, AAVH-1/hu.1, AAVH2, AAVH-5/hu.3, AAVH6, AAVhE1.1, AAVhEr1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVLG-10/rh.40, AAVLG-4/rh.38, AAVLG-9/hu.39, AAVLG-9/hu.39, AAV-LKO1, AAV-LK02, AAV-LK03, AAV-LK03, AAV-LK04, AAV-LK05, AAV-LK06, AAVLK07, AAV-LK09, AAV-LK10, AAV-LK11, AAV-LK12, AAV-LK13, AAV-LK14, AAV-LK16, AAV-LK17, AAVLK18, AAV-LK19, AAVN721-8/rh.43, AAV-PAEC, AAVPAEC12, AAV-PAEC11, AAV-PAEC2, AAV-PAEC4, AAVPAEC6, AAV-PAEC7, AAV-PAECS, Anc80, Anc80L65, Anc81, Anc82, Anc83, Anc84, Anc94, Anc110, Anc113, Anc126, Anc127, BAAV, BNP61 AAV, BNP62 AAV, BNP63 AAV, bovine AAV, caprine AAV, Japanese AAV10 serotype, UPENN AAV10, VOY101, and VOY201.

In certain embodiments, an AAV serotypes and/or pseudotype is AAV-DJ/8, AAV9, AAV Php.B, and/or AAV Php.eB. In certain embodiments, an AAV serotype and/or pseudotype comprises AAV-DJ/8. In certain embodiments, an AAV serotype and/or pseudotype comprises AAV9. In certain embodiments, an AAV serotype and/or pseudotype comprises AAV Php.B. In certain embodiments, an AAV serotype and/or pseudotype comprises AAV Php.eB.

In certain embodiments, an AAV is an AAV variant that has been genetically modified, e.g., by substitution, deletion or addition of one or several amino acid residues in one or more capsid proteins. Examples of such variants include, but are not limited to, AAV2 with one or more of Y444F, Y500F, Y730F and/or S662V mutations; AAV3 with one or more of Y705F, Y731F and/or T492V mutations; and AAV6 with one or more of S663V and/or T492V mutations.

In certain embodiments, an AAV capsid is modified to comprise at least one surface-bound saccharide or a derivative thereof. As used herein, the term “surface-bound”, when referring to the at least one saccharide, means that said at least one saccharide is bound to and exposed at the outer surface of the AAV vector. Suitable examples of saccharides include, but are not limited to, monosaccharides, oligosaccharides, polysaccharides, and derivatives thereof.

### 2. AAV Constructs

In certain embodiments, the present disclosure provides polynucleotide vectors (e.g., polynucleotide constructs) that comprise a nucleotide sequence encoding a gene product for gene therapy or a characteristic portion thereof. In certain embodiments described herein, a polynucleotide vector comprising a gene product for gene therapy is a polynucleotide construct, and can be comprised in an AAV capsid to produce a recombinant AAV particle (e.g., a recombinant AAV particle comprises a polynucleotide construct comprised in an AAV capsid).

In certain embodiments, a polynucleotide construct comprises one or more components derived from or modified from a naturally occurring AAV genomic construct. In certain embodiments, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV DJ/8 construct, an AAV9 construct, an AAV2.7m8 construct, an AAV8BP2 construct, an AAV293 construct, an AAVPhp.B construct, or AAVPhp.eB construct (see e.g., Chan et al., 2017, Nat Neuroscience 20(8):1172-9). Additional exemplary AAV constructs that can be used herein are known in the art. See, e.g., Kanaan et al., Mol. Ther. Nucleic Acids 8: 184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which are incorporated herein by reference for the purposes described herein).

In certain embodiments, AAV derived sequences (e.g., which are comprised in a polynucleotide construct) typically include the cis-acting 5′ and 3′ inverted terminal repeat (ITR) sequences (see, e.g., B. J. Carter, in “Handbook of Parvoviruses,” ed., P. Tijsser, CRC Press, pp. 155 168, 1990, which is incorporated herein by reference for the purposes described herein). Typical AAV2-derived ITR sequences are about 145 nucleotides in length. In some embodiments, at least or exactly 80% of a typical ITR sequence (e.g., at least or exactly 85%, at least or exactly 90%, at least or exactly 95%, or at least or exactly 100%, etc.) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated herein by reference for the purposes described herein). In certain embodiments, any of the coding sequences and/or polynucleotide constructs described herein are flanked by 5′ and 3′ AAV ITR sequences. The AAV ITR sequences may be obtained from any known AAV, including presently identified AAV types.

In certain embodiments, polynucleotide constructs described in accordance with this disclosure and in a pattern known to the art (see, e.g., Asokan et al., Mal. Ther. 20: 699-7080, 2012, which is incorporated herein by reference for the purposes described herein) are typically comprised of, a coding sequence or a portion thereof, at least one and/or control sequence, and optionally 5′ and 3′ AAV inverted terminal repeats (ITRs). In certain embodiments, provided constructs can be packaged into a capsid to create an AAV particle. An AAV particle may be delivered to a selected target cell. In certain embodiments, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In certain embodiments, a nucleic acid coding sequence is operatively linked to and/or control components in a manner that permits coding sequence transcription, translation, and/or expression in a cell of a target tissue.

In certain embodiments, an unmodified AAV endogenous genome includes two open reading frames, “cap” and “rep,” which are flanked by ITRs. In certain embodiments, recombinant AAV constructs similarly comprise one or more open reading frames flanked by ITR sequences. In certain embodiments, an AAV construct also comprises conventional control elements that are operably linked to the coding sequence in a manner that permits its transcription, translation and/or expression in a cell transfected with the polynucleotide construct or infected with a virus particle produced by the disclosure. In certain embodiments, an AAV construct comprises a promoter, an enhancer, an untranslated region (e.g., a 5′ UTR, 3′ UTR), a Kozak sequence, an internal ribosomal entry site (IRES), splicing sites (e.g., an acceptor site, a donor site), a polyadenylation site, and/or any combination thereof.

In certain embodiments, a construct is an AAV construct. In certain embodiments, an AAV construct can include at least 500 bp, at least 1 kb, at least 1.5 kb, at least 2 kb, at least 2.5 kb, at least 3 kb, at least 3.5 kb, at least 4 kb, or at least 4.5 kb. In some embodiments, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some embodiments, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In certain embodiments, a promoter can be a native promoter, a constitutive promoter, an inducible promoter, and/or a tissue-specific promoter. Non-limiting examples of control sequences are described herein and others are known in the art.

### 3. AAV Capsids

In certain embodiments, the present disclosure provides one or more polynucleotide constructs packaged into an AAV capsid. In certain embodiments, an AAV capsid is from or is derived from an AAV capsid of an AAV2, 3, 4, 5, 6, 7, 8, 9, 10, 11, 12, rh8, rh10, rh39, rh43 or Ancestral serotype, or one or more hybrids thereof. In certain embodiments, an AAV capsid is from an AAV ancestral serotype. In certain embodiments, an AAV capsid is an ancestral (Ane) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. In certain embodiments, an AAV capsid is engineered and/or derived from an AAV9 capsid. In certain embodiments, an AAV capsid is an AAV PHP.eB capsid. In certain embodiments, an AAV capsid is an AAV PHP.B capsid (see e.g., Diptaman Chatterjee et al., Gene Therapy 29, 390-397 (2022)).

As provided herein, in certain embodiments, any combination of AAV capsids and AAV constructs (e.g., comprising AAV ITRs) may be used in recombinant AAV particles of the present disclosure. For example, but not limited to, wild type or variant AAV2 ITRs and AAV DJ/8, AAV9, AAV PHP.B, and/or AAV PHP.eB capsid, or variant AAV2 ITRs and AAV6 capsid, etc. In certain embodiments of the present disclosure, an AAV particle is wholly comprised of AAV2 and/or AAV9 components (e.g., capsid and ITRs are AAV2 and/or AAV9 serotype). In certain embodiments, an AAV particle is an AAV2/6, AAV2/8 or AAV2/9 particle (e.g., an AAV6, AAV8 or AAV9 capsid with an AAV construct having AAV2 ITRs).

### 4. Exemplary AAV Construct Components

a. Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated herein by reference for the purposes described herein). Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV particle.

An AAV particle of the present disclosure can comprise an AAV construct comprising a coding sequence (e.g., a gene product for gene therapy) and associated elements flanked by a 5′ and a 3′ AAV ITR sequences. In certain embodiments, an ITR is or comprises about 130 nucleic acids. In certain embodiments, an ITR is or comprises about 145 nucleic acids. In certain embodiments, all or substantially all of a sequence encoding an ITR is used. In certain embodiments, an AAV ITR sequence may be obtained from any known AAV, including presently identified mammalian AAV types. In certain embodiments an ITR is an AAV2 ITR. In certain embodiments, an ITR is an AAV9 ITR.

A non-limiting example of a polynucleotide construct of the present disclosure is a “cis acting” construct comprising a transgene, in which said transgene sequence and any associated regulatory elements are flanked by 5′ or “left” and 3′ or “right” AAV ITR sequences. 5′ and left designations refer to a position of an ITR sequence relative to an entire construct, read left to right, in a sense direction. In certain embodiments, a 5′ or left ITR is an ITR that is closest to a promoter (e.g., as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence (e.g., as opposed to a promoter sequence) for a given construct, when a construct is depicted in a sense orientation, linearly. In general, TTRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to anti sense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

In certain embodiments, an ITR (e.g., a 5′ ITR) can have a sequence according to SEQ ID NO: 27. In certain embodiments, an ITR (e.g., a 3′ ITR) can have a sequence according to SEQ ID NO: 28. In certain embodiments, an ITR includes one or more modifications, e.g., truncations, deletions, substitutions or insertions, as is known in the art. In certain embodiments, an ITR comprises fewer than 145 nucleotides, for example, but not limited to, 120, 121, 122, 123, 124, 125, 126, 127, 128, 129, 130, 131, 132, 133, 134, 135, or 141 nucleotides. In certain embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides.

In certain embodiments, a 5′ ITR sequence is at least or exactly 85%, 90%, 95%, 96%, 97%, 98%, 99%, or 100% identical to a 5′ ITR sequence represented by SEQ ID NO: 27. In certain embodiments, a 3′ ITR sequence is at least or exactly 85%, 90%, 95%, 96%, 97%, 98%, 99%, or 100% identical to a 3′ ITR sequence represented by SEQ ID NO: 28.

SEQ ID NO: 27 - Exemplary 5′ AAV ITR nucleotide
sequence
(SEQ ID NO: 27)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT
SEQ ID NO: 28 - Exemplary 3′ AAV ITR nucleotide
sequence
(SEQ ID NO: 28)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG

b. Promoters

In certain embodiments, a construct (e.g., an AAV construct) comprises a promoter. The term “promoter” refers to a DNA sequence recognized by enzymes/proteins that can promote and/or initiate transcription of an operably linked gene (e.g., a gene product for gene therapy). For example, a promoter typically refers to, e.g., a nucleotide sequence to which an RNA polymerase and/or any associated factor binds and from which it can initiate transcription. Thus, in certain embodiments, a construct (e.g., an AAV construct) comprises a promoter operably linked to one of the non-limiting example promoters described herein.

In certain embodiments, a promoter is an inducible promoter, a constitutive promoter, a mammalian cell promoter, a viral promoter, a chimeric promoter, an engineered promoter, a tissue-specific promoter, or any other type of promoter known in the art. In certain embodiments, a promoter is a RNA polymerase II promoter, such as a mammalian RNA polymerase II promoter. In certain embodiments, a promoter is a RNA polymerase III promoter, including, but not limited to, a HI promoter, a human U6 promoter, a mouse U6 promoter, or a swine U6 promoter.

A variety of promoters are known in the art, which in certain embodiments, can be used herein. Nonlimiting examples of promoters that can be used herein in some embodiments include: human EF1α, human cytomegalovirus (CMV) (U.S. Pat. No. 5,168,062, which is incorporated herein by reference for the purposes described herein), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), a-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GAL V) promoters. Additional examples of promoters are known in the art. See, e.g., Lodish, Molecular Cell Biology, Freeman and Company, New York 2007, each of which is incorporated herein by reference for the purposes described herein. In certain embodiments, a promoter is the CMV immediate early promoter. In certain embodiments, the promoter is a CAG promoter and/or a CAG/CBA promoter.

The term “constitutive” promoter refers to a nucleotide sequence that, when operably linked with a nucleic acid encoding a protein, causes RNA to be transcribed from the nucleic acid in a cell under most or all physiological conditions. Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated herein by reference for the purposes described herein), the SV 40 promoter, the dihydrofolate reductase promoter, the beta-actin promoter, the phosphoglycerol kinase (PGK) promoter, the EF1-alpha promoter (Invitrogen), or any other constitutive promoters known in the art.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art. Examples of inducible promoters regulated by exogenously supplied compounds include the zinc-inducible sheep metallothionein (MT) promoter, the dexamethasone (Dex) inducible mouse mammary tumor virus (MMTV) promoter, the T7 polymerase promoter system (see e.g., WO 98/10088, which is incorporated herein by reference for the purposes described herein); the ecdysone insect promoter (see e.g., No et al., Proc. Natl. Acad Sci. U.S.A. 93:3346-3351, 1996, which is incorporated herein by reference for the purposes described herein), the tetracycline-repressible system (see e.g., Gossen et al., Proc. Natl. Acad Sci. U.S.A. 89:5547-5551, 1992, which is incorporated herein by reference for the purposes described herein), the tetracycline-inducible system (see e.g., Gossen et al., Science 268: 1766-1769, 1995, see also Harvey et al., Curr. Opin. Chem. Biol. 2:512-518, 1998, each of which is incorporated herein by reference for the purposes described herein), the RU486-inducible system (see e.g., Wang et al., Nat. Biotech. 15:239-243, 1997, and Wang et al., Gene Ther. 4:432-441, 1997, each of which is incorporated herein by reference for the purposes described herein), and the rapamycin-inducible system (see e.g., Magari et al., J Clin. Invest. 100:2865-2872, 1997, which is incorporated herein by reference for the purposes described herein).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter). In certain embodiments, regulatory and/or control sequences impart tissue-specific gene expression capabilities. In certain cases, tissue-specific regulatory and/or control sequences bind tissue-specific transcription factors that induce transcription in a tissue-specific manner.

SEQ ID NO: 29 - Exemplary CAG promoter nucleotide
sequence
(SEQ ID NO: 29)
TCGAGGTGAGCCCCACGTTCTGCTTCACTCTCCCCATCTCCCCCCCCTCC
CCACCCCCAATTTTGTATTTATTTATTTTTTAATTATTTTGTGCAGCGAT
GGGGGCGGGGGGGGGGGGGGGGCGCGCGCCGGGGGGGGGGGGGGGGGGGG
GGGGGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAA
AAGCG
SEQ ID NO: 30 - Exemplary CAG promoter/enhancer
nucleotide sequence
(SEQ ID NO: 30)
TCGAGGTGAGCCCCACGTTCTGCTTCACTCTCCCCATCTCCCCCCCCTCC
CCACCCCCAATTTTGTATTTATTTATTTTTTAATTATTTTGTGCAGCGAT
GGGGGCGGGGGGGGGGGGGGGGCGCGCGCCGGGGGGGGGGGGGGGGGGGG
GGGGGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAA
AAGCGAAGCGCGCGGCGGGCGGGAGTCGCTGCGCGCTGCCTTCGCCCCGT
GCCCCGCTCCGCCGCCGCCTCGCGCCGCCCGCCCCGGCTCTGACTGACCG
CGTTACTCCCACAGGTGAGCGGGCGGGACGGCCCTTCTCCTCCGGGCTGT
AATTAGCGCTTGGTTTAATGACGGCTTGTTTCTTTTCTGTGGCTGCGTGA
AAGCCTTGAGGGGCTCCGGGAGGGCCCTTTGTGCGGGGGGAGCGGCTCGG
GGCTGTCCGCGGGGGGACGGCTGCCTTCGGGGGGGACGGGGCAGGGGGGG
GTTCGGCTTCTGGCGTGTGACCGGCGGCTCTAGAGCCTCTGCTAACCATG
TTCATGCCTTCTTCTTTTTCCTACAGCTCCTGGGCAACGTGCTGGTTATT
GTGCTGTCTCATCATTTTGGCAAAGAATTGGATCCGCCACC

c. Enhancers

In certain embodiments, a construct can include an enhancer sequence. The term “enhancer” as used herein refers to a nucleotide sequence that can increase the level of transcription of a nucleic acid encoding a protein of interest (e.g., a gene product for gene therapy), and/or increase or modify the translational efficiency of a transcript following transcription. In certain embodiments, enhancer sequences (generally 50-1500 bp in length) generally increase the level of transcription by providing additional binding sites for transcription-associated proteins (e.g., transcription factors), and/or stabilize or modify post-transcriptional regulatory machinery. In certain embodiments, an enhancer sequence is found within an intronic sequence. In certain embodiments, an enhancer sequence is found in a 3′ and/or 5′ UTR. In certain embodiments, an enhancer region is found downstream of a coding sequence comprising a transgene and proximal to a polyadenylation sequence. Unlike promoter sequences, enhancer sequences can act at much larger distance away from the transcription start site (e.g., as compared to a promoter). Non-limiting examples of enhancers include a woodchuck hepatitis virus post-transcriptional regulatory element (WPRE), RSV enhancer, a CMV enhancer, and/or a SV40 enhancer.

SEQ ID NO: 31 - Exemplary WPRE nucleotide sequence
(SEQ ID NO: 31)
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGC

d. Flanking Untranslated Regions, 5′ UTR and 3′ UTR

In certain embodiments, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, particles, polynucleotides, compositions, kits, or methods as described herein to enhance or otherwise modulate the expression of a gene product for gene therapy.

Natural 5′ UTRs include a sequence that plays a role in translation initiation. In certain embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In certain embodiments, 5′ UTRs also form secondary structures that are involved in elongation factor binding. In certain embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (see e.g., Chen et al., Mol. Cell. Biol. 15:5777-5788, 1995; Chen et al., Mol. Cell Biol. 15:2010-2018, 1995, each of which is incorporated herein by reference for the purposes described herein): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger RNA, whereas members of the ELAV family, most notably HuR, have been documented to increase the stability of mRNA. HuR binds to AREs of all the three classes. Engineering the HuR specific binding sites into the 3′ UTR of nucleic acid molecules may lead to HuR binding and thus, stabilization of the RNA.

In certain embodiments, the introduction, removal, or modification of 3′ UTR AREs can be used to modulate the stability of an mRNA encoding a gene product for gene therapy. In certain embodiments, AREs can be removed or mutated to increase the intracellular stability and thus increase translation and production of a gene for gene therapy.

In certain embodiments, non-ARE sequences may be incorporated into the 5′ or 3′ UTRs. In certain embodiments, introns or portions of intron sequences may be incorporated into the flanking regions of the polynucleotides in any of the constructs, particles, polynucleotides, compositions, kits, and methods provided herein. Incorporation of intronic sequences may increase protein production as well as mRNA levels.

e. Internal Ribosome Entry Sites (IRES)

In certain embodiments, a construct encoding a gene product for gene therapy can include an internal ribosome entry site (IRES). An IRES forms a complex secondary structure that allows translation initiation to occur from any position with an mRNA immediately downstream from where the IRES is located (see, e.g., Pelletier and Sonenberg, Mol. Cell. Biol. 8(3): 1103-1112, 1988, which is incorporated herein by reference for the purposes described herein). There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HA V), hepatitis C virus (HCV), and poliovirus (PV) (see e.g., Alberts, Molecular Biology of the Cell, Garland Science, 2002; and Hellen et al., Genes Dev. 15(13):1593-612, 2001, each of which are incorporated herein by reference for the purposes described herein).

In certain embodiments, an IRES sequence that is incorporated into a construct that encodes gene product for gene therapy may be the foot and mouth disease virus (FMDV) 2A sequence. The Foot and Mouth Disease Virus 2A sequence is a small peptide (approximately 18 amino acids in length) that has been shown to mediate the cleavage of polyproteins (see e.g., Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated herein by reference for the purposes described herein). The cleavage activity of the 2A sequence has previously been demonstrated in artificial systems including plasmids and gene therapy constructs (e.g., AAV and retroviruses) (see e.g., Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6: 198-208, 1999; de Felipe et al., Human Gene Therapy II: 1921-1931, 2000; and Klump et al., Gene Therapy 8:811-817, 2001, each of which is incorporated herein by reference for the purposes described herein).

In certain embodiments, an IRES can be utilized in an AAV construct. In some embodiments, a construct encoding a gene product for gene therapy can include a polynucleotide internal ribosome entry site (IRES). In certain embodiments, an IRES can be part of a composition comprising more than one construct. In certain embodiments, an IRES is used to produce more than one polypeptide from a single gene transcript.

f. Splice Sites

In certain embodiments, any of the constructs provided herein can include splice donor and/or splice acceptor sequences, which are functional during RNA processing occurring during transcription. In certain embodiments, splice sites are involved in trans-splicing.

g. Polyadenylation Sequences

In certain embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference for the purposes described herein). A poly(A) tail confers mRNA stability and transferability (see e.g., Molecular Biology of the Cell, Third Edition by B. Alberts et al., Garland Publishing, 1994, which is incorporated herein by reference for the purposes described herein). In some embodiments, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., about 50, 60, 70, 100, 200, 500, 1000, 2000, 3000, 4000, or 5000) added to the pre-mRNA through the action of an enzyme, polyadenylate polymerase. In certain embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used in some embodiments, including those derived from bovine growth hormone (bGH) (Woychik et al., Proc. Natl. Acad Sci. U.S.A. 81(13):3944-3948, 1984; U.S. Pat. No. 5,122,458, each of which is incorporated herein by reference for the purposes described herein), mouse-p-globin, mouse-a-globin (Orkin et al., EMBO J 4(2):453-456, 1985; Thein et al., Blood 71(2):313-319, 1988, each of which is incorporated herein by reference for the purposes described herein), human collagen, polyoma virus (Batt et al., Mol. Cell Biol. 15(9):4783-4790, 1995, which is incorporated herein by reference for the purposes described herein), the Herpes simplex virus thymidine kinase gene (HSV TK), IgG heavy-chain gene polyadenylation signal (US 2006/0040354, which is incorporated herein by reference for the purposes described herein), human growth hormone (hGH) (Szymanski et al., Mol Therapy 15(7):1340-1347, 2007, which is incorporated herein by reference for the purposes described herein), and/or the group consisting of SV40 poly(A) site, such as the SV40 late and early poly(A) site (see e.g., Schek et al., Mol Cell Biol. 12(12):5386-5393, 1992, which is incorporated herein by reference for the purposes described herein).

In certain embodiments, the poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference for the purposes described herein). In certain embodiments, a poly(A) signal sequence can be a synthetic polyadenylation site (see, e.g., the pCl-neo expression construct of Promega that is based on Levitt et al., Genes Dev. 3(7):1019-1025, 1989, which is incorporated herein by reference for the purposes described herein).

SEQ ID NO: 32 - Exemplary SV40 poly A signal
nucleotide sequence
(SEQ ID NO: 32)
TAAGATACATTGATGAGTTTGGACAAACCACAACTAGAATGCAGTGAAAA
AAATGCTTTATTTGTGAAATTTGTGATGCTATTGCTTTATTTGTAACCAT
TATAAGCTGCAATAAACAAGTT

h. Additional Sequences

In certain embodiments, constructs of the present disclosure may comprise a 2A element or sequence. In certain embodiments, constructs of the present disclosure may include one or more cloning sites. In certain embodiments, cloning sites may not be fully removed prior to manufacturing for administration to a subject. In certain embodiments, cloning sites may have functional roles including as linker sequences, or as portions of a Kozak site. As will be appreciated by those skilled in the art, cloning sites may vary significantly in primary sequence while retaining their desired function.

In certain embodiments, a 2A element is a T2A, P2A, E2A, and/or F2A element. In certain embodiments, a 2A sequence may comprise a 5′ linker sequence, for example, but not limited to GSG (e.g., Glycine, Serine, Glycine).

SEQ ID NO: 33 - Exemplary T2A element amino acid
sequence
(SEQ ID NO: 33)
EGRGSLLTCGDVEENPGP
SEQ ID NO: 34 - Exemplary P2A element amino acid
sequence
(SEQ ID NO: 34)
ATNFSLLKQAGDVEENPGP
SEQ ID NO: 35 - Exemplary E2A element amino acid
sequence
(SEQ ID NO: 35)
QCTNYALLKLAGDVESNPGP
SEQ ID NO: 36 - Exemplary F2A element amino acid
sequence
(SEQ ID NO: 36)
VKQTLNFDLLKLAGDVESNPGP
SEQ ID NO: 37 - Exemplary P2A element nucleotide
sequence
(SEQ ID NO: 37)
CTCGAGGCCACCAACTTCTCCCTGCTGAAGCAGGCCGGCGACGTGGAGGA
GAACCCCGGCCCC

i. Destabilization Domains

In certain embodiments, any of the constructs provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays, fluorescent activating cell sorting (FACS) assays, and/or immunological assays (e.g., enzyme linked immunosorbent assay (ELISA), radioimmunoassay (RIA), and immunohistochemistry).

Additional examples of destabilizing sequences are known in the art. In certain embodiments, the destabilizing sequence is a FK506- and rapamycin-binding protein (FKBP12) sequence, and the stabilizing ligand is Shield-I (Shld1) (see e.g., Banaszynski et al. (2012) Cell 126(5):995-1004, which is incorporated herein by reference for the purposes described herein). In certain embodiments, a destabilizing sequence is a DHFR sequence, and a stabilizing ligand is trimethoprim (TMP) (see e.g., Iwamoto et al., (2010) Chem Biol 17:981-988, which is incorporated herein by reference for the purposes described herein).

j. Reporter Sequences or Elements

In certain embodiments, constructs provided herein can optionally include a sequence encoding a reporter polypeptide and/or protein (“a reporter sequence”). Non-limiting examples of reporter sequences include DNA sequences encoding: a beta-lactamase, a betagalactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays, fluorescent activating cell sorting (FACS) assays and/or immunological assays (e.g., enzyme linked immunosorbent assay (ELISA), radioimmunoassay (RIA), and immunohistochemistry).

In certain embodiments, a reporter sequence is the LacZ gene, and the presence of a construct carrying the LacZ gene in a cell is detected by assays for beta-galactosidase activity. In certain embodiments, a reporter sequence is a fluorescent protein (e.g., green fluorescent protein (GFP)) or luciferase. In certain embodiments where a reporter sequence is a fluorescent protein or luciferase, the presence of a construct carrying the fluorescent protein or luciferase in a cell may be measured by fluorescent imaging techniques (e.g., fluorescent microscopy or FACS) or light production in a luminometer (e.g., a spectrophotometer or an IVIS imaging instrument). In certain embodiments, a reporter sequence can be used to verify tissue-specific targeting capabilities and/or tissue-specific promoter regulatory and/or control activity of any of the constructs described herein. An exemplary GFP tag sequence is provided as SEQ ID NO: 39.

In certain embodiments, a reporter sequence is a FLAG tag (e.g., a 3×FLAG tag), and the presence of a construct carrying the FLAG tag in a cell is detected by protein binding or detection assays (e.g., Western blots, immunohistochemistry, radioimmunoassay (RIA), mass spectrometry). An exemplary 3×FLAG tag sequence is provided as SEQ ID NO: 38.

SEQ ID NO: 38 - Exemplary 3xFLAG tag nucleotide
sequence
(SEQ ID NO: 38)
GGATCCCGGGCTGACTACAAAGACCATGACGGTGATTATAAAGATCATGA
CATCGACTACAAGGATGACGATGACAAG
SEQ ID NO: 39 - Exemplary GFP nucleotide sequence
(SEQ ID NO: 39)
ATGGTGAGCAAGGGCGAGGAGCTGTTCACCGGGGGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATTTGCACCA
CCGGCAAGCTGCCCGTGCCCTGGCCCACCCTCGTGACCACCCTGACCTAC
GGCGTGCAGTGCTTCAGCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACG
TCTATATCATGGCCGACAAGCAGAAGAACGGCATCAAGGTGAACTTCAAG
ATCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAG

### 5. Virus Purification

Recombinant AAV particles are produced in permissive (packaging) host cell cultures. Typically, cell supernatant and/or cell lysates are used to release rAAV particles and maximize yield of recovered rAAV. However, the supernatants and/or cell lysate contains various cellular components such as host cell DNA, host cell proteins, media components, and in some instances, helper virus or helper virus nucleic acids, which must be separated from the rAAV vector before it is suitable for in vivo use. Advances in rAAV production include the use of non-adherent cell suspension processes in stirred tank bioreactors and production conditions whereby rAAV vectors are released into the media or supernatant reducing the concentration of host cellular components present in the production material but still containing appreciable amounts of in-process impurities (see e.g., U.S. Pat. No. 6,566,118 and PCT WO 99/11764). In certain embodiments, rAAV particles may be collected from the media and/or cell lysate and further purified.

Methods of rAAV purification are known in the art and may be used for the purification of rAAV produced using the present disclosure. Examples of purification methods include, but are not limited to, lysate clarification, cesium chloride gradient centrifugation, iodixanol gradient separation, diafiltration, chromatography purification, and/or any combination thereof.

In certain embodiments, the methods of the present disclosure comprise an ultracentrifugation step during which a density gradient is formed. Though not wishing to be bound to a theory, it is believed that the ultracentrifugation step allows for full AAV capsids to be separated from empty AAV capsids. Examples of ultracentrifugation protocols can be found in, for example, but not limited to, WO2008135229, which is incorporated herein in its entirety for the purposes described herein.

The methods of the present disclosure may comprise yet other additional steps, which may further increase the purity of the AAV and remove other unwanted components and/or concentrate the fraction and/or condition the fraction for a subsequent step.

In certain embodiments, the method comprises a depth filtration step. In certain embodiments, the method comprises subjecting a fraction of a transfected cell culture supernatant to depth filtration using a filter comprising cellulose and perlites and having a minimum permeability of about 500 L/m². In certain embodiments, the method further comprises use of a filter having a minimum pore size of about 0.2 μm. In certain embodiments, the depth filtration is followed by filtration through the filter having a minimum pore size of about 0.2 μm. In certain embodiments, one or both of the depth filter and filter having a minimum pore size of about 0.2 μm are washed and the washes are collected. In certain embodiments, the washes are pooled together and combined with the filtrate obtained upon depth filtration and filtration with the filter having a minimum pore size of about 0.2 pico-meters. In certain embodiments, the depth filtration step and other filtration step occurs prior to the ultracentrifugation step described herein.

In certain embodiments, the methods of the present disclosure comprise one or more chromatography steps. In certain embodiments, the methods comprise a negative chromatography step whereby unwanted components bind to the chromatography resin and the desired AAV does not bind to the chromatography resin. In certain embodiments, the methods comprise a negative anion exchange (AEX) chromatography step, or an AEX chromatography step in the “non-binding mode”. Advantages of “non-binding mode” include relative ease of carrying out the procedure and in conducting subsequent assaying. Accordingly, in certain embodiments, the methods of purifying AAV particles comprise performing negative anion exchange (AEX) chromatography on a fraction comprising AAV particles by applying the fraction to an AEX chromatography column or membrane under conditions that allow for the AAV to flow through the AEX chromatography column or membrane and collecting AAV particles. In certain embodiments, the fraction is applied to the AEX chromatography column or membrane with a loading buffer comprising about 100 mM to about 150 mM salt, e.g., NaCl, optionally, wherein the pH of the loading buffer is about 8 to about 9. In certain embodiments, the loading buffer comprises about 115 mM to about 130 mM salt, e.g., NaCl, optionally, wherein the loading buffer comprises about 120 mM to about 125 mM salt, e.g., NaCl. In certain embodiments, the negative AEX step occurs prior to the ultracentrifugation step described herein.

In certain embodiments, the methods of the present disclosure comprise concentrating an AAV fraction using an ultra/diafiltration system. In certain embodiments, the methods of the present disclosure comprise one more tangential flow filtration (TFF) steps. In certain embodiments, the AAV fraction undergoes ultra/diafiltration. In certain embodiments, the AAV fraction is concentrated with the ultra/diafiltration system before a step comprising performing negative AEX chromatography, after a step comprising performing negative AEX chromatography, or before and after comprising performing negative AEX chromatography.

In certain embodiments, the methods of the present disclosure comprise treating a fraction comprising AAV particles with a solvent detergent to inactivate lipid enveloped viruses.

In certain embodiments, the methods of the present disclosure comprise filtration of a fraction comprising rAAV particles to remove viruses of greater size than the rAAV particles in the fraction. In certain embodiments, the method of the present disclosure comprises filtration of a fraction comprising AAV to remove viruses sized greater than or about 35 nm. In certain embodiments, the pore size of the filter is in the nanometer range, and, in certain embodiments, the method comprises nanofiltration. In certain embodiments, the method of the present disclosure comprises use of a nanofilter of pore size in the range of 35 nanometer±2 nanometer, as determined by a water flow method. Classification of the type of filter is dependent on membrane structure, material, and vendor.

In certain embodiments, during the filtration step, a pressure difference over the filter is maintained. In certain embodiments, the pressure (pressure drop across the filter) is about 0.02 MN to about 0.1 MPa. In certain embodiments, the pressure (e.g., pressure drop across the filter) is about 0.02 MPa to about 0.08 MPa. In case the filter is run in dead-end mode, the pressure difference can be affected by the feed pressure of the sample applied (i.e., by adjustment of a pump to a specific flow, which affects the feed pressure).

In certain embodiments, the filtration step for removal of viruses larger than the rAAV particles occurs once during the process of the present disclosure. In certain embodiments, the filtration step occurs twice during the process. In certain embodiments, the filtration step for removal of viruses larger than the rAAV particles occurs after the ultracentrifugation step described herein. In certain embodiments, the filtration step for removal of viruses larger than the rAAV particles occurs after a polish step.

In certain embodiments, the methods of the present disclosure comprise a polish step comprising performing AEX chromatography, optionally with a column comprising tentacle gel.

### 6. Titer Assaying

Generally, methods for assaying for empty capsids and AAV vector particles with packaged genomes have been known in the art. See, e.g., Grimm et al., Gene Therapy (1999) 6:1322-1330; Sommer et al., Malec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, for example, but not limited to, an anti-AAV capsid monoclonal antibody, for example, but not limited to, the B1 anti-AAV-2 monoclonal antibody (Wobus et al., J. Viral. (2000), 9281-9293). A secondary antibody is then used, one that binds to the primary antibody and contains a means for detecting binding with the primary antibody, more preferably an anti-IgG antibody containing a detection molecule covalently bound to it, for example, but not limited to, a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, colorimetric changes, chemiluminescence, and/or fluorescent detection kit. For example, for SDS-PAGE, samples from column fractions can be taken and heated in SDS-PAGE loading buffer containing reducing agent (e.g., DTT), and capsid proteins resolved on pre-cast gradient polyacrylamide gels (e.g., Novex). Silver staining may be performed using SilverXpress (Invitrogen, Calif.) according to the manufacturer's instructions.

In certain embodiments, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector may be employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, or alternatively, droplet digital PCR (ddPCR) may be used. For example, methods for determining single-stranded and self-complementary AAV vector genome titers by ddPCR have been described (see, e.g., M. Lock et al, Hum Gene Ther Methods. 2014 April; 25(2): 15-25).

In certain embodiments, the method comprises testing an AAV preparation via an AAV-specific ELISA assay to quantify the total number of AAV capsids in the AAV preparation. The number of capsids can then be used to determine the dose to administer a subject. In certain embodiments, the method comprises testing an AAV preparation via an AAV-specific ELISA assay in combination with a method that evaluates or confirms the percentage or ratio of full versus empty (full:empty) AAV capsids in the AAV preparation. Methods of quantifying AAV particles using ELISA are known in the art, as described in US 2020/0018752, which is incorporated herein in its entirety for the purpose described herein.

### K. Compositions

In certain embodiments, compositions described herein can comprise products generated through a method described herein. In some embodiments, compositions described herein may be comprised in a formulation with one or more additional therapeutic agents. In certain embodiments, compositions described herein may be comprised in a formulation wherein the formulation comprises pharmaceutically acceptable excipients.

In certain embodiments, constructs, particles, polypeptides, polynucleotides, and/or compositions described herein may be comprised.

In certain embodiments, compositions described herein may be administered to a subject in need thereof, such as but not limited to, a subject with a genetic disorder, infection, autoimmune disease, and/or cancer.

In certain embodiments, a subject is a mammal. In certain embodiments, a subject is a domestic animal. In certain embodiments, a subject is a farm animal. In certain embodiments, a subject is a zoo animal. In certain embodiments, a subject is a dog or a cat. In certain embodiments, a subject is a cow, a horse, a sheep, or a goat. In certain embodiments, a subject can be, but is not limited to, a dog, cat, ferret, rabbit, cow, duck, pig, goat, chicken, horse, llama, camel, ostrich, deer, turkey, dove, sheep, goose, oxen, and/or reindeer. In certain embodiments, a subject is a human. In certain embodiments, a subject is equal to, less than, or greater than 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age.

In certain embodiments, compositions described herein and/or additional therapeutic agents are administered to cells in vitro. In certain embodiments, compositions described herein and/or additional therapeutic agents are administered intravenously, intramuscularly, subcutaneously, topically, orally, transdermally, intraperitoneally, intraorbitally, by implantation, by inhalation, intrathecally, intraventricularly, intranasally, and/or any combinations thereof. In certain embodiments, compositions described herein and/or additional therapeutic agents are administered intravenously, intramuscularly, subcutaneously, topically, orally, transdermally, intraperitoneally, intraorbitally, by implantation, by inhalation, intrathecally, intraventricularly, intranasally, and/or any combinations thereof. In certain embodiments, an appropriate dosage of compositions described herein may be determined based on the type of disease to be treated, severity and course of the disease, the clinical condition of the individual, the individual's clinical history and response to the treatment, and/or the discretion of the attending physician.

In certain embodiments, the compositions comprising virions produced as described herein can comprise an amount of packaged virions in the range of about 1.0×10⁹ to 1.0×10¹⁶ including all integers or fractional amounts within the range. In certain embodiments, compositions can comprise about 1.0×10¹² to 1.0×10¹⁴ packaged virions. In certain embodiments, the compositions are formulated to contain at least about 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹¹, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ packaged virions, or any range derivable therein.

### VI. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

A standard recombinant AAV (rAAV) construct, comprising a promoter, transgene, and polyadenylation signal placed between the 145 nucleotide (nt) ITRs of AAV is used to introduce a specific transgenes into a virus. To circumvent the problems with delivery of rep and cap in trans, rep and cap RNAs are utilized. RNAs for gene products required from adenovirus, which includes E1, E2, and/or E4 are also utilized.

The rAAV is made by transfection of host cells (e.g., 293 cells) first with rAAV and RNAs encoding Rep78, Rep68, Rep52, and/or Rep40, followed later by cap proteins VP1, VP2, and/or VP3. The adenovirus RNAs E2 and/or E4 are transfected with the RNAs encoding rep gene products, and E1 product is supplied by the transgenic host cells (e.g., 293 cells). Specific infectivity of rAAV prepared using the AAV rep and cap, but using transfection on alternative cell lines (e.g., 84-31 cells, a derivative of 293 cells) that provide E4 adenoviral help in trans is also compared.

AAV rep, and AAV cap encoding polynucleotides, and adenovirus protein encoding polynucleotides are cloned into specific plasmids comprising a T7 RNA polymerase promoter start site and poly adenylation site. Linear RNAs are produced. Linear RNAs are purified using an HPLC method. Synthetic RNAs (rep) are generated, and doses of polynucleotides at 0.1, 0.25, 0.5 and 1 ug/100,000 cells are transfected into host cells (e.g., 293 cells) along with various combinations of adenovirus helper genes. AAV rep gene product encoding polynucleotides, rAAV plasmid, and adenovirus RNAs are transfected on day 0, and then cap gene product encoding RNA are added 0, 1, 2, 3, 4, and/or 5 days after the original transfections. The optimum time window for delivery of cap gene product encoding polynucleotides is determined. rAAV is harvested from the cells approximately 1-2 weeks later. Crude rAAV preparations are tested to determine how adjustment of variables such as delivery timing, concentrations, and combinations improve rAAV production.

TABLE 1
Groups used for RNA testing

| | Rep | Cap | E1 | E2 | E4 | |
|---|---|---|---|---|---|---|
| | RNA | RNA | RNA | RNA | RNA | Plasmid |
| Group 1 | | | | | | X |
| Group 2 | | | | | | X |
| Group 3 | X | X | | | | |
| Group 4 | X | X | X | X | X | |
| Group 5 | X | X | X | | | |
| Group 6 | X | X | | X | | |
| Group 7 | X | X | | | X | |
| Group 8 | X | X | X | X | | |
| Group 9 | X | X | | X | X | |
| Group 10 | X | X | X | | X | |

Standard linear RNAs are utilized, which have stable translation for approximately 48 hrs. If some RNAs, such as adenovirus helper RNAs, need to persist longer, pseudo-uridine modifications are utilized to increase the RNA lifespan in cells. If additional stability is required, circular RNAs are utilized. The optimal exposure time length & amount of RNA within the cells is determined to provide optimal rAAV yield. Improved complementation, rAAV yield, and/or rAAV configuration is observed in bands using density gradients. Western blots are used to determine transfection success & rates for the various RNAs. Lipid nanoparticles (cationic or ionizable) can be utilized for improved delivery.

rep68 synthetic RNA and/or rep78 synthetic RNA are delivered to cells containing a plasmid with AAV ITRs flanking an expression cassette (e.g., a GFP expression cassette, etc). A significant increase in expression cassette product (e.g., transcription and/or translation product(s)) is observed (e.g., as determined by a suitable method, such as FACS).

rep78 synthetic RNA was delivered (transfected) into host cells one hour after transfection of a plasmid containing AAV ITRs flanking a GFP expression cassette. A significant 18-fold increase in GFP signal was observed relative to controls when determined by FACs. The results showed that synthetic rep78 RNA made rep78 protein that was able to replicate the plasmid DNA containing the AAV sequences.

All of the methods disclosed and claimed herein can be made and executed without undue experimentation in light of the present disclosure. While the compositions and methods of this invention have been described in terms of particular embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.
Muzyczka N. Use of adeno-associated virus as a general transduction vector for mammalian cells. Curr Top Microbiol Immunol. 1992; 158:97-129. doi: 10.1007/978-3-642-75608-5_5. PMID: 1316261.
Cheung A K, Hoggan M D, Hauswirth W W, Berns K I. Integration of the adeno-associated virus genome into cellular DNA in latently infected human Detroit 6 cells. J Virol. 1980 February; 33(2):739-48. doi: 10.1128/JVI.33.2.739-748.1980. PMID: 6251245; PMCID: PMC288599.
Muzyczka, 1991, “In Vitro Replication of Adeno-Associated Virus DNA”, Virol. 2:281-290.
Pereira, et al., 1997, “The adeno-associated virus (AAV) Rep protein acts as both a repressor and an activator to regulate AAV transcription during a productive infection.” J Virol. 71(2):1079-88.
Tratschin et al., 1986, “Negative and Positive in trans of Gene Expression from Adeno-Associated Virus Vectors on Mammalian Cells by a Viral rep Gene Product”, Mol. Cell. Biol. 6:2884-2894.
Antoni et al., 1991, “Adeno-Associated Virus Rep Protein Inhibits Human Immunodeficiency Virus Type 1 Production in Human Cells”, J. Virol. 65:396-404.
Heilbronn et al., 1990, “The Adeno-Associated Virus rep Gene Suppresses Herpes Simplex Virus-Induced DNA Amplification”, J. Virol. 64:3012-3018.
Hermonat, 1994, “Down-Regulation of the Human c-fos and c-myc Proto-Oncogene Promoters by Adeno-Associated Virus Rep78”, Cancer Lett. 81:129-136.
Horer et al., 1995, “Mutational Analysis of Adeno-Associated Virus Rep Protein-Mediated Inhibition of Heterologous and Homologous Promoters”, J. Virol. 69:5485-5496.
Labow et al., 1987, “Adeno-Associated Virus Gene Expression Inhibits Cellular Transformation by Heterologous Genes”, Mol. Cell. Biol. 7:1320-1325.
Lewis et al., 1995, “Adenovirus E1A Proteins Interact with the Cellular YY1 Transcription Factor”, J. Virol 69, 1628-1636.
Shi Y, Seto E, Chang L S, Shenk T. Transcriptional repression by YY1, a human GLI-Kruppel-related protein, and relief of repression by adenovirus E1A protein. Cell. 1991 Oct. 18; 67(2):377-88. doi: 10.1016/0092-8674(91)90189-6. PMID: 1655281.
Labow M A, Berns K I. The adeno-associated virus rep gene inhibits replication of an adeno-associated virus/simian virus 40 hybrid genome in cos-7 cells. J Virol. 1988 May; 62(5):1705-12. doi: 10.1128/JVI.62.5.1705-1712.1988. PMID: 2833621; PMCID: PMC253208.
Li J, Samulski R J, Xiao X. Role for highly regulated rep gene expression in adeno-associated virus vector production. J Virol. 1997 July; 71(7):5236-43. doi: 10.1128/JVI.71.7.5236-5243.1997. PMID: 9188591; PMCID: PMC191759.
Meier A F, Fraefel C, Seyffert M. The Interplay between Adeno-Associated Virus and its Helper Viruses. Viruses. 2020 Jun. 19; 12(6):662. doi: 10.3390/v12060662. PMID: 32575422; PMCID: PMC7354565.
Nguyen T N T, Sha S, Hong M S, Maloney A J, Barone P W, Neufeld C, Wolfrum J, Springs S L, Sinskey A J, Braatz R D. Mechanistic model for production of recombinant adeno-associated virus via triple transfection of HEK293 cells. Mol Ther Methods Clin Dev. 2021 Apr. 16; 21:642-655. doi: 10.1016/j.omtm.2021.04.006. PMID: 34095346; PMCID: PMC8143981.
Srivastava A, Lusby E W, Berns K I. Nucleotide sequence and organization of the adeno-associated virus 2 genome. J Virol. 1983 February; 45(2):555-64. doi: 10.1128/JVI.45.2.555-564.1983. PMID: 6300419; PMCID: PMC256449.
Muzyczka N. Use of adeno-associated virus as a general transduction vector for mammalian cells. Curr Top Microbiol Immunol. 1992; 158:97-129. doi: 10.1007/978-3-642-75608-5_5. PMID: 1316261.
Ruffing M, Zentgraf H, Kleinschmidt J A. Assembly of viruslike particles by recombinant structural proteins of adeno-associated virus type 2 in insect cells. J Virol. 1992 December; 66(12):6922-30. doi: 10.1128/JVI.66.12.6922-6930.1992. PMID: 1331503; PMCID: PMC240317.
Ausubel F. M. et al., eds., 1989, Current Protocols in Molecular Biology, Vol. I, Green Publishing Associates, Inc., and John Wiley & sons, Inc., New York.
Hu M C, Davidson N. The inducible lac operator-repressor system is functional in mammalian cells. Cell. 1987 Feb. 27; 48(4):555-66. doi: 10.1016/0092-8674(87)90234-0. PMID: 3028641.
Brown M, Figge J, Hansen U, Wright C, Jeang K T, Khoury G, Livingston D M, Roberts T M. lac repressor can regulate expression from a hybrid SV40 early promoter containing a lac operator in animal cells. Cell. 1987 Jun. 5; 49(5):603-12. doi: 10.1016/0092-8674(87)90536-8. PMID: 3034429.
Melefors O, Hentze M W. Translational regulation by mRNA/protein interactions in eukaryotic cells: ferritin and beyond. Bioessays. 1993 February; 15(2):85-90. doi: 10.1002/bies.950150203. PMID: 8471060.
Geall A J, Mandl C W, Ulmer J B. RNA: the new revolution in nucleic acid vaccines. Semin Immunol. 2013 April; 25(2):152-9. doi: 10.1016/j.smim.2013.05.001. Epub 2013 Jun. 2. PMID: 23735226.
Brunelle J L, Green R. In vitro transcription from plasmid or PCR-amplified DNA. Methods Enzymol. 2013; 530:101-14. doi: 10.1016/B978-0-12-420037-1.00005-1. PMID: 24034317.
Kern J A, Davis R H. Application of solution equilibrium analysis to in vitro RNA transcription. Biotechnol Prog. 1997 November-December; 13(6):747-56. doi: 10.1021/bp970094p. PMID: 9413132.
Kern J A, Davis R H. Application of a fed-batch system to produce RNA by in vitro transcription. Biotechnol Prog. 1999 March-April; 15(2):174-84. doi: 10.1021/bp990008g. PMID: 10194392.
WO 1995/008626, Marble and Davis (March 1995).
Guzaev A P. Solid-phase supports for oligonucleotide synthesis. Curr Protoc Nucleic Acid Chem. 2013 June; Chapter 3:Unit3.1. doi: 10.1002/0471142700.nc0301s53. PMID: 23775808.
Borovkov A Y, Loskutov A V, Robida M D, Day K M, Cano J A, Le Olson T, Patel H, Brown K, Hunter P D, Sykes K F. High-quality gene assembly directly from unpurified mixtures of microarray-synthesized oligonucleotides. Nucleic Acids Res. 2010 October; 38(19):e180. doi: 10.1093/nar/gkg677. Epub 2010 Aug. 6. PMID: 20693531; PMCID: PMC2965257.
Sindelar L E, Jaklevic J M. High-throughput DNA synthesis in a multichannel format. Nucleic Acids Res. 1995 Mar. 25; 23(6):982-7. doi: 10.1093/nar/23.6.982. PMID: 7731813; PMCID: PMC306795.
U.S. Pat. No. 6,653,468, Guzaev and Manoharan (November 2011)
Greene, et al. “Protective Groups in Organic Synthesis”, 2d. Ed., Wiley & Sons, 1991.
Abramova T. Frontiers and approaches to chemical synthesis of oligodeoxyribonucleotides. Molecules. 2013 Jan. 15; 18(1):1063-75. doi: 10.3390/molecules18011063. PMID: 23322070; PMCID: PMC6269945.
U.S. Pat. No. 8,309,707, Srivastava et al. (November 2012).
US Pat. Publication No. 2013/0072670, Srivastava et al. (March 2013).
PCT Pat. Application No. WO2013123125, Church et al. (August 2013).
Pon R T, Yu S, Guo Z, Sanghvi Y S. Multiple oligodeoxyribonucleotide syntheses on a reusable solid-phase CPG support via the hydroquinone-O,O′-diacetic acid (Q-Linker) linker arm. Nucleic Acids Res. 1999 Mar. 15; 27(6):1531-8. doi: 10.1093/nar/27.6.1531. PMID: 10037817; PMCID: PMC148349.
Nagata S, Hamasaki T, Uetake K, Masuda H, Takagaki K, Oka N, Wada T, Ohgi T, Yano J. Synthesis and biological activity of artificial mRNA prepared with novel phosphorylating reagents. Nucleic Acids Res. 2010 November; 38(21):7845-57. doi: 10.1093/nar/gkg638. Epub 2010 Jul. 26. PMID: 20660478; PMCID: PMC2995060.
Shiba Y, Masuda H, Watanabe N, Ego T, Takagaki K, Ishiyama K, Ohgi T, Yano J. Chemical synthesis of a very long oligoribonucleotide with 2-cyanoethoxymethyl (CEM) as the 2′-O-protecting group: structural identification and biological activity of a synthetic 110mer precursor-microRNA candidate. Nucleic Acids Res. 2007; 35(10):3287-96. doi: 10.1093/nar/gkm202. Epub 2007 Apr. 25. PMID: 17459888; PMCID: PMC1904286.
U.S. Pat. No. 8,309,706, Dellinger et al. (November 2011).
U.S. Pat. No. 8,242,258, Dellinger et al. (August 2012).
U.S. Pat. No. 8,202,983, Dellinger et al. (June 2012).
U.S. Pat. No. 7,999,087, Dellinger et al. (August 2011).
U.S. Pat. No. 7,667,033, Alvarado (February 2010).
U.S. Pat. No. 5,889,136, Scaringe et al. (March 1999).
U.S. Publication No. 2008/0119645, Griffey et al. (May 2008).
U.S. Publication No. 2011/0275793, Debart et al. (November 2011).
U.S. Pat. No. 7,476,709, Moody et al. (January 2009).
WO Publication No. 2013/052523, Fourgerolles et al. (April 2013).
Stepinski J, Waddell C, Stolarski R, Darzynkiewicz E, Rhoads R E. Synthesis and properties of mRNAs containing the novel “anti-reverse” cap analogs 7-methyl(3′-O-methyl)GpppG and 7-methyl (3′-deoxy)GpppG. RNA. 2001 October; 7(10):1486-95. PMID: 11680853; PMCID: PMC1370192.
U.S. Pat. No. 7,074,596, Darzynkiewicz et al. (July 2006).
WO Publication No. 2008/016473, Kore et al. (February 2008).
WO Publication No. 2008/157688, Jemielity et al. (December 2008).
WO Publication No. 2009/149253, Kowalska et al. (December 2009).
WO Publication No. 2011/015347, Sahin et al. (February 2011).
WO Publication No. 2013/059475, Kore et al. (April 2013).
U.S. Publication No. 2019/0010485 A1, Yazdan Panah et al. (January 2019).
Kore A R, Xiao Z, Li M. Synthesis and biological validation of N⁷-(4-chlorophenoxyethyl) substituted dinucleotide cap analogs for mRNA translation. Bioorg Med Chem. 2013 Aug. 1; 21(15):4570-4. doi: 10.1016/j.bmc.2013.05.041. Epub 2013 Jun. 1. PMID: 23777824.
Mahon K P, Love K T, Whitehead K A, Qin J, Akinc A, Leshchiner E, Leshchiner I, Langer R, Anderson D G. Combinatorial approach to determine functional group effects on lipidoid-mediated siRNA delivery. Bioconjug Chem. 2010 Aug. 18; 21(8):1448-54. doi: 10.1021/bc100041r. PMID: 20715849: PMCID: PMC2931596.
Schroeder A, Levins C G, Cortez C, Langer R, Anderson D G. Lipid-based nanotherapeutics for siRNA delivery. J Intern Med. 2010 January; 267(1):9-21. doi: 10.1111/j.1365-2796.2009.02189.x. PMID: 20059641; PMCID: PMC5308083.
Akinc A, Zumbuehl A, Goldberg M, Leshchiner E S, Busini V, Hossain N, Bacallado S A, Nguyen D N, Fuller J, Alvarez R, Borodovsky A, Borland T, Constien R, de Fougerolles A, Dorkin J R, Narayanannair Jayaprakash K, Jayaraman M, John M, Koteliansky V, Manoharan M, Nechev L, Qin J, Racie T, Raitcheva D, Rajeev K G, Sah D W, Soutschek J, Toudjarska I, Vornlocher H P, Zimmermann T S, Langer R, Anderson D G. A combinatorial library of lipid-like materials for delivery of RNAi therapeutics. Nat Biotechnol. 2008 May; 26(5):561-9. doi: 10.1038/nbt1402. Epub 2008 Apr. 27. PMID: 18438401; PMCID: PMC3014085.
Love K T, Mahon K P, Levins C G, Whitehead K A, Querbes W, Dorkin J R, Qin J, Cantley W, Qin L L, Racie T, Frank-Kamenetsky M, Yip K N, Alvarez R, Sah D W, de Fougerolles A, Fitzgerald K, Koteliansky V, Akinc A, Langer R, Anderson D G. Lipid-like materials for low-dose, in vivo gene silencing. Proc Natl Acad Sci USA. 2010 Feb. 2; 107(5):1864-9. doi: 10.1073/pnas.0910603106. Epub 2010 Jan. 11. Erratum in: Proc Natl Acad Sci USA. 2010 May 25; 107(21):9915. PMID: 20080679; PMCID: PMC2804742.
Siegwart D J, Whitehead K A, Nuhn L, Sahay G, Cheng H, Jiang S, Ma M, Lytton-Jean A, Vegas A, Fenton P, Levins C G, Love K T, Lee H, Cortez C, Collins S P, Li Y F, Jang J, Querbes W, Zurenko C, Novobrantseva T, Langer R, Anderson D G. Combinatorial synthesis of chemically diverse core-shell nanoparticles for intracellular delivery. Proc Natl Acad Sci USA. 2011 Aug. 9; 108(32):12996-3001. doi: 10.1073/pnas.1106379108. Epub 2011 Jul. 22. PMID: 21784981; PMCID: PMC3156157.
Frank-Kamenetsky M, Grefhorst A, Anderson N N, Racie T S, Bramlage B, Akinc A, Butler D, Charisse K, Dorkin R, Fan Y, Gamba-Vitalo C, Hadwiger P, Jayaraman M, John M, Jayaprakash K N, Maier M, Nechev L, Rajeev K G, Read T, Röhl I, Soutschek J, Tan P, Wong J, Wang G, Zimmermann T, de Fougerolles A, Vornlocher H P, Langer R, Anderson D G, Manoharan M, Koteliansky V, Horton J D, Fitzgerald K. Therapeutic RNAi targeting PCSK9 acutely lowers plasma cholesterol in rodents and LDL cholesterol in nonhuman primates. Proc Natl Acad Sci USA, 2008 Aug. 19; 105(33):11915-20. doi: 10.1073/pnas.0805434105. Epub 2008 Aug. 11. PMID: 18695239; PMCID: PMC2575310.
Akinc A, Goldberg M, Qin J, Dorkin J R, Gamba-Vitalo C, Maier M, Jayaprakash K N, Jayaraman M, Rajeev K G, Manoharan M, Koteliansky V, Röhl I, Leshchiner E S, Langer R, Anderson D G. Development of lipidoid-siRNA formulations for systemic delivery to the liver. Mol Ther. 2009 May; 17(5):872-9. doi: 10.1038/mt.2009.36. Epub 2009 Mar. 3. PMID: 19259063; PMCID: PMC2835134.
Leuschner F, Dutta P, Gorbatov R, Novobrantseva T I, Donahoe J S, Courties G, Lee K M, Kim J I, Markmann J F, Marinelli B, Panizzi P, Lee W W, Iwamoto Y, Milstein S, Epstein-Barash H, Cantley W, Wong J, Cortez-Retamozo V, Newton A, Love K, Libby P, Pittet M J, Swirski F K, Koteliansky V, Langer R, Weissleder R, Anderson D G, Nahrendorf M. Therapeutic siRNA silencing in inflammatory monocytes in mice. Nat Biotechnol. 2011 Oct. 9; 29(11):1005-10. doi: 10.1038/nbt.1989. PMID: 21983520; PMCID: PMC3212614.
Murugaiah V, Zedalis W, Lavine G, Charisse K, Manoharan M. Reversed-phase high-performance liquid chromatography method for simultaneous analysis of two liposome-formulated short interfering RNA duplexes. Anal Biochem. 2010 Jun. 1; 401(1):61-7. doi: 10.1016/j.ab.2010.02.012. Epub 2010 Feb. 13. PMID: 20156415.
U.S. Pat. No. 8,450,298, Mahon et al. (May 2013).
U.S. Publication No. US20130177638, Schutt et al. (July 2013).
U.S. Publication No. US20130177637, Schutt et al. (July 2013).
U.S. Publication No. US20130177636, Schutt et al (July 2013).
U.S. Publication No. US20130177635, Schutt et al. (July 2013).
U.S. Publication No. US20130177634, Schutt et al. (July 2013).
U.S. Publication No. US20130177633, Schutt et al. (July 2013).
U.S. Publication No. US20130183375, Schutt et al. (July 2013).
U.S. Publication No. US20130183373, Schutt et al. (July 2013).
U.S. Publication No. US20130183372, Schutt et al. (July 2013).
U.S. Publication No. US20100324120, Chen et al. (December 2010).
Wheeler J J, Palmer L, Ossanlou M, MacLachlan I, Graham R W, Zhang Y P, Hope M J, Scherrer P, Cullis P R. Stabilized plasmid-lipid particles: construction and characterization. Gene Ther. 1999 February; 6(2):271-81. doi: 10.1038/sj.gt.3300821. PMID: 10435112.
Zhang Y P, Sekirov L, Saravolac E G, Wheeler J J, Tardi P, Clow K, Leng E, Sun R, Cullis P R, Scherrer P. Stabilized plasmid-lipid particles for regional gene therapy: formulation and transfection properties. Gene Ther. 1999 August; 6(8):1438-47. doi: 10.1038/sj.gt.3300965. PMID: 10467368.
Jeffs L B, Palmer L R, Ambegia E G, Giesbrecht C, Ewanick S, MacLachlan I. A scalable, extrusion-free method for efficient liposomal encapsulation of plasmid DNA. Pharm Res. 2005 March; 22(3):362-72. doi: 10.1007/s11095-004-1873-z. PMID: 15835741.
Morrissey D V, Lockridge J A, Shaw L, Blanchard K, Jensen K, Breen W, Hartsough K, Machemer L, Radka S, Jadhav V, Vaish N, Zinnen S, Vargeese C, Bowman K, Shaffer C S, Jeffs L B, Judge A, MacLachlan I, Polisky B. Potent and persistent in vivo anti-HBV activity of chemically modified siRNAs. Nat Biotechnol. 2005 August; 23(8):1002-7. doi: 10.1038/nbt1122. Epub 2005 Jul. 24. PMID: 16041363.
Zimmermann T S, Lee A C, Akinc A, Bramlage B, Bumcrot D, Fedoruk M N, Harborth J, Heyes J A, Jeffs L B, John M, Judge A D, Lam K, McClintock K, Nechev L V, Palmer L R, Racie T, Röhl I, Seiffert S, Shanmugam S, Sood V, Soutschek J, Toudjarska I, Wheat A J, Yaworski E, Zedalis W, Koteliansky V, Manoharan M, Vornlocher H P, MacLachlan I. RNAi-mediated gene silencing in non-human primates. Nature. 2006 May 4; 441(7089):111-4. doi: 10.1038/nature04688. Epub 2006 Mar. 26. PMID: 16565705.
Heyes J, Palmer L, Bremner K, MacLachlan I. Cationic lipid saturation influences intracellular delivery of encapsulated nucleic acids. J Control Release. 2005 Oct. 3; 107(2):276-87. doi: 10.1016/j.jconrel.2005.06.014. PMID: 16054724.
Semple S C, Akinc A, Chen J, Sandhu A P, Mui B L, Cho C K, Sah D W, Stebbing D, Crosley E J, Yaworski E, Hafez I M, Dorkin J R, Qin J, Lam K, Rajeev K G, Wong K F, Jeffs L B, Nechev L, Eisenhardt M L, Jayaraman M, Kazem M, Maier M A, Srinivasulu M, Weinstein M J, Chen Q, Alvarez R, Barros S A, De S, Klimuk S K, Borland T, Kosovrasti V, Cantley W L, Tam Y K, Manoharan M, Ciufolini M A, Tracy M A, de Fougerolles A, MacLachlan I, Cullis P R, Madden T D, Hope M J. Rational design of cationic lipids for siRNA delivery. Nat Biotechnol. 2010 February; 28(2):172-6. doi: 10.1038/nbt.1602. Epub 2010 Jan. 17. PMID: 20081866.
Judge A D, Robbins M, Tavakoli I, Levi J, Hu L, Fronda A, Ambegia E, McClintock K, MacLachlan I. Confirming the RNAi-mediated mechanism of action of siRNA-based cancer therapeutics in mice. J Clin Invest. 2009 March; 119(3):661-73. doi: 10.1172/JC137515. Epub 2009 Feb. 23. PMID: 19229107; PMCID: PMC2648695.
de Fougerolles A R. Delivery vehicles for small interfering RNA in vivo. Hum Gene Ther. 2008 February; 19(2):125-32. doi: 10.1089/hum.2008.928. PMID: 18257677.
U.S. Publication No. US20130122104, Yaworski et al. (May 2013).
PCT Publication No. WO201087791, Dardas et al. (August 2010).
PCT Publication No. WO2011076807, Baryza et al. (June 2011).
U.S. Publication No. US20110200582, Baryza et al. (August 2011).
U.S. Publication No. US20120177724, Irvine et al. (July 2012).
PCT Publication No. WO2013086526, Kesari et al. (June 2013).
Landen C N, Merritt W M, Mangala L S, Sanguino A M, Bucana C, Lu C, Lin Y G, Han L Y, Kamat A A, Schmandt R, Coleman R L, Gershenson D M, Lopez-Berestein G, Sood A K. Intraperitoneal delivery of liposomal siRNA for therapy of advanced ovarian cancer. Cancer Biol Ther. 2006 December; 5(12):1708-13. doi: 10.4161/cbt.5.12.3468. Epub 2006 Dec. 30. PMID: 17106249.
U.S. Publication No. US20130090372, Budzik et al. (April 2013).
PCT Publication No. WO2013006825, Geall et al. (January 2013).
U.S. Publication No. US20120178702, Huang et al. (July 2012).
PCT Publication No. WO2012013326, Baumhof et al. (February 2012).
U.S. Publication No. US20130142818, Baumhof et al. (June 2013).
Semple S C, Akinc A, Chen J, Sandhu A P, Mui B L, Cho C K, Sah D W, Stebbing D, Crosley E J, Yaworski E, Hafez I M, Dorkin J R, Qin J, Lam K, Rajeev K G, Wong K F, Jeffs L B, Nechev L, Eisenhardt M L, Jayaraman M, Kazem M, Maier M A, Srinivasulu M, Weinstein M J, Chen Q, Alvarez R, Barros S A, De S, Klimuk S K, Borland T, Kosovrasti V, Cantley W L, Tam Y K, Manoharan M, Ciufolini M A, Tracy M A, de Fougerolles A, MacLachlan I, Cullis P R, Madden T D, Hope M J. Rational design of cationic lipids for siRNA delivery. Nat Biotechnol. 2010 February; 28(2):172-6. doi: 10.1038/nbt.1602. Epub 2010 Jan. 17. PMID: 20081866.
Basha G, Novobrantseva T I, Rosin N, Tam Y Y, Hafez I M, Wong M K, Sugo T, Ruda V M, Qin J, Klebanov B, Ciufolini M, Akinc A, Tam Y K, Hope M J, Cullis P R. Influence of cationic lipid composition on gene silencing properties of lipid nanoparticle formulations of siRNA in antigen-presenting cells. Mol Ther. 2011 December; 19(12):2186-200. doi: 10.1038/mt.2011.190. Epub 2011 Oct. 4. PMID: 21971424; PMCID: PMC3242662.
PCT Publication No. WO2011127255, Burke et al. (October 2011).
PCT Publication No. WO2008103276, Jadhav et al. (August 2008).
Yoon C S, Park J H. Ultrasound-mediated gene delivery. Expert Opin Drug Deliv. 2010 March; 7(3):321-30. doi: 10.1517/17425241003596329. PMID: 20166854.
Postema M, Gilja O H. Ultrasound-directed drug delivery. Curr Pharm Biotechnol. 2007 December; 8(6):355-61. doi: 10.2174/138920107783018453. PMID: 18289044.
Newman C M, Bettinger T. Gene therapy progress and prospects: ultrasound for gene transfer. Gene Ther. 2007 March; 14(6):465-75. doi: 10.1038/sj.gt.3302925. PMID: 17339881.
U.S. Publication No. US20100196983, Yang et al. (August 2010).
U.S. Publication No. US20100009424, Forde et al. (January 2010).
Andre F M, Mir L M. Nucleic acids electrotransfer in vivo: mechanisms and practical aspects. Curr Gene Ther. 2010 August; 10(4):267-80. doi: 10.2174/156652310791823380. PMID: 20557285.
Chiarella P, Fazio V M, Signori E. Application of electroporation in DNA vaccination protocols. Curr Gene Ther. 2010 August; 10(4):281-6. doi: 10.2174/156652310791823506. PMID: 20504275.
Hojman P. Basic principles and clinical advancements of muscle electrotransfer. Curr Gene Ther. 2010 April; 10(2):128-38. doi: 10.2174/156652310791110994. PMID: 20222860.
E P Publication No. EP 0779931, Wurm et al. (June 1997).
Weiss and Frock, “Rapid Analysis of Particle Size Distribution by Laser Light Scattering,” 1976, Powder Technology, 14: 287.
Molecular Cloning, 3^{rd} edition, Sambrook and Russell, Cold Spring Harbor Laboratory Press, 2001.
Asokan A, Schaffer D V, Samulski R J. The AAV vector toolkit: poised at the clinical crossroads. Mol Ther. 2012 April; 20(4):699-708. doi: 10.1038/mt.2011.287. Epub 2012 Jan. 24. PMID: 22273577; PMCID: PMC3321598.
U.S. Publication No. US20150050243, Kaczmarczyk et al. (February 2015).
U.S. Publication No. US20090305344, Polo et al. (December 2009).
U.S. Publication No. US20060177819, Smith et al. (August 2008).
Chan K Y, Jang M J, Yoo B B, Greenbaum A, Ravi N, Wu W L, Sanchez-Guardado L, Lois C, Mazmanian S K, Deverman B E, Gradinaru V. Engineered AAVs for efficient noninvasive gene delivery to the central and peripheral nervous systems. Nat Neurosci. 2017 August; 20(8):1172-1179. doi: 10.1038/nn.4593. Epub 2017 Jun. 26. PMID: 28671695; PMCID: PMC5529245.
Kanaan N M, Sellnow R C, Boye S L, Coberly B, Bennett A, Agbandje-McKenna M, Sortwell C E, Hauswirth W W, Boye S E, Manfredsson F P. Rationally Engineered AAV Capsids Improve Transduction and Volumetric Spread in the CNS. Mol Ther Nucleic Acids. 2017 Sep. 15; 8:184-197. doi: 10.1016/j.omtn.2017.06.011. Epub 2017 Jun. 21. PMID: 28918020; PMCID: PMC5503098.
Li W, Asokan A, Wu Z, Van Dyke T, DiPrimio N, Johnson J S, Govindaswamy L, Agbandje-McKenna M, Leichtle S, Redmond D E Jr, McCown T J, Petermann K B, Sharpless N E, Samulski R J. Engineering and selection of shuffled AAV genomes: a new strategy for producing targeted biological nanoparticles. Mol Ther. 2008 July; 16(7):1252-60. doi: 10.1038/mt.2008.100. Epub 2008 May 20. PMID: 18500254; PMCID: PMC2632803.
Adachi K, Enoki T, Kawano Y, Veraz M, Nakai H. Drawing a high-resolution functional map of adeno-associated virus capsid by massively parallel sequencing. Nat Commun. 2014; 5:3075. doi: 10.1038/ncomms4075. PMID: 24435020; PMCID: PMC3941020.
Isgrig K, McDougald D S, Zhu J, Wang H J, Bennett J, Chien W W. AAV2.7m8 is a powerful viral vector for inner ear gene therapy. Nat Commun. 2019 Jan. 25; 10(1):427. doi: 10.1038/s41467-018-08243-1. PMID: 30683875; PMCID: PMC6347594.
Gao G, Vandenberghe L H, Alvira M R, Lu Y, Calcedo R, Zhou X, Wilson J M. Clades of Adeno-associated viruses are widely disseminated in human tissues. J Virol. 2004 June; 78(12):6381-8. doi: 10.1128/JVI.78.12.6381-6388.2004. PMID: 15163731; PMCID: PMC416542.
B. J. Carter, in “Handbook of Parvoviruses,” ed., P. Tijsser, CRC Press, 1990.
Sambrook et al., “Molecular Cloning. A Laboratory Manual”, 2d ed., Cold Spring Harbor Laboratory, New York, 1989
Fisher K J, Gao G P, Weitzman M D, DeMatteo R, Burda J F, Wilson J M. Transduction with recombinant adeno-associated virus for gene therapy is limited by leading-strand synthesis. J Virol. 1996 January; 70(1):520-32. doi: 10.1128/JVI.70.1.520-532.1996. PMID: 8523565; PMCID: PMC189840.
Asokan A, Schaffer D V, Samulski R J. The AAV vector toolkit: poised at the clinical crossroads. Mol Ther. 2012 April; 20(4):699-708. doi: 10.1038/mt.2011.287. Epub 2012 Jan. 24. PMID: 22273577; PMCID: PMC3321598.
Chatterjee D, Marmion D J, McBride J L, Manfredsson F P, Butler D, Messer A, Kordower J H. Enhanced CNS transduction from AAV.PHP.eB infusion into the cisterna magna of older adult rats compared to AAV9. Gene Ther. 2022 June; 29(6):390-397. doi: 10.1038/s41434-021-00244-y. Epub 2021 Mar. 22. PMID: 33753910; PMCID: PMC9203269.
B. J. Carter, in “Handbook of Parvoviruses”, ed., P. Tijsser, CRC Press, 1990.
U.S. Publication No. U.S. Pat. No. 5,168,062, Stinski (December 2012).
Boshart M, Weber F, Jahn G, Dorsch-Hasler K, Fleckenstein B, Schaffner W. A very strong enhancer is located upstream of an immediate early gene of human cytomegalovirus. Cell. 1985 June; 41(2):521-30. doi: 10.1016/s0092-8674(85)80025-8. PMID: 2985280.
PCT Publication No. WO 98/10088, Wilson et al. (March 1998).
No D, Yao T P, Evans R M. Ecdysone-inducible gene expression in mammalian cells and transgenic mice. Proc Natl Acad Sci USA. 1996 Apr. 16; 93(8):3346-51. doi: 10.1073/pnas.93.8.3346. PMID: 8622939; PMCID: PMC39610.
Gossen M, Bujard H. Tight control of gene expression in mammalian cells by tetracycline-responsive promoters. Proc Natl Acad Sci USA. 1992 Jun. 15; 89(12):5547-51. doi: 10.1073/pnas.89.12.5547. PMID: 1319065; PMCID: PMC49329.
Gossen M, Freundlieb S, Bender G, Müller G, Hillen W, Bujard H. Transcriptional activation by tetracyclines in mammalian cells. Science. 1995 Jun. 23; 268(5218):1766-9. doi: 10.1126/science.7792603. PMID: 7792603.
Harvey D M, Caskey C T. Inducible control of gene expression: prospects for gene therapy. Curr Opin Chem Biol. 1998 August; 2(4):512-8. doi: 10.1016/s1367-5931(98)80128-2. PMID: 9736925.
Wang Y, DeMayo F J, Tsai S Y, O'Malley B W. Ligand-inducible and liver-specific target gene expression in transgenic mice. Nat Biotechnol. 1997 March; 15(3):239-43. doi: 10.1038/nbt0397-239. PMID: 9062922.
Wang Y, Xu J, Pierson T, O'Malley B W, Tsai S Y. Positive and negative regulation of gene expression in eukaryotic cells with an inducible transcriptional regulator. Gene Ther. 1997 May; 4(5):432-41. doi: 10.1038/sj.gt.3300402. PMID: 9274720.
Magari S R, Rivera V M, Iuliucci J D, Gilman M, Cerasoli F Jr. Pharmacologic control of a humanized gene therapy system implanted into nude mice. J Clin Invest. 1997 Dec. 1; 100(11):2865-72. doi: 10.1172/JCI119835. PMID: 9389753; PMCID: PMC508493.
Chen C Y, Xu N, Shyu A B. mRNA decay mediated by two distinct A U-rich elements from c-fos and granulocyte-macrophage colony-stimulating factor transcripts: different deadenylation kinetics and uncoupling from translation. Mol Cell Biol. 1995 October; 15(10):5777-88. doi: 10.1128/MCB.15.10.5777. PMID: 7565731; PMCID: PMC230830.
Chen Q, Adams C C, Usack L, Yang J, Monde R A, Stern D B. An A U-rich element in the 3′ untranslated region of the spinach chloroplast petD gene participates in sequence-specific RNA-protein complex formation. Mol Cell Biol. 1995 April; 15(4):2010-18. doi: 10.1128/MCB.15.4.2010. PMID: 7891696; PMCID: PMC230428.
Pelletier J, Kaplan G, Racaniello V R, Sonenberg N. Cap-independent translation of poliovirus mRNA is conferred by sequence elements within the 5′ noncoding region. Mol Cell Biol. 1988 March; 8(3):1103-12. doi: 10.1128/mcb.8.3.1103-1112.1988. PMID: 2835660; PMCID: PMC363253.
Alberts, Molecular Biology of the Cell, Garland Science, 2002
Hellen C U, Sarnow P. Internal ribosome entry sites in eukaryotic mRNA molecules. Genes Dev. 2001 Jul. 1; 15(13):1593-612. doi: 10.1101/gad.891101. PMID: 11445534.
Ryan M D, Drew J. Foot-and-mouth disease virus 2A oligopeptide mediated cleavage of an artificial polyprotein. EMBO J. 1994 Feb. 15; 13(4):928-33. doi: 10.1002/j.1460-2075.1994.tb06337.x. PMID: 8112307; PMCID: PMC394894.
Mattion N M, Harnish E C, Crowley J C, Reilly P A. Foot-and-mouth disease virus 2A protease mediates cleavage in attenuated Sabin 3 poliovirus vectors engineered for delivery of foreign antigens. J Virol. 1996 November; 70(11):8124-7. doi: 10.1128/JVI.70.11.8124-8127.1996. PMID: 8892938; PMCID: PMC190887.
Furler S, Paterna J C, Weibel M, Biieler H. Recombinant AAV vectors containing the foot and mouth disease virus 2A sequence confer efficient bicistronic gene expression in cultured cells and rat substantia nigra neurons. Gene Ther. 2001 June; 8(11):864-73. doi: 10.1038/sj.gt.3301469. PMID: 11423934.
Halpin C, Cooke S E, Barakate A, El Amrani A, Ryan M D. Self-processing 2A-polyproteins—a system for co-ordinate expression of multiple proteins in transgenic plants. Plant J. 1999 February; 17(4):453-9. doi: 10.1046/j.1365-313x.1999.00394.x. PMID: 10205902.
de Felipe P, Martin V, Cortés M L, Ryan M, Izquierdo M. Use of the 2A sequence from foot-and-mouth disease virus in the generation of retroviral vectors for gene therapy. Gene Ther. 1999 February; 6(2):198-208. doi: 10.1038/sj.gt.3300811. PMID: 10435104.
De Felipe P, Izquierdo M. Tricistronic and tetracistronic retroviral vectors for gene transfer. Hum Gene Ther. 2000 Sep. 1; 11(13):1921-31. doi: 10.1089/10430340050129530. PMID: 10986564.
Klump H, Schiedlmeier B, Vogt B, Ryan M, Ostertag W, Baum C. Retroviral vector-mediated expression of HoxB4 in hematopoietic cells using a novel coexpression strategy. Gene Ther. 2001 May; 8(10):811-7. doi: 10.1038/sj.gt.3301447. PMID: 11420646.
Proudfoot N J, Furger A, Dye M J. Integrating mRNA processing with transcription. Cell. 2002 Feb. 22; 108(4):501-12. doi: 10.1016/s0092-8674(02)00617-7. PMID: 11909521.
Molecular Biology of the Cell, Third Edition by B. Alberts et al., Garland Publishing, 1994.
Woychik R P, Lyons R H, Post L, Rottman F M. Requirement for the 3 flanking region of the bovine growth hormone gene for accurate polyadenylylation. Proc Natl Acad Sci USA. 1984 July; 81(13):3944-8. doi: 10.1073/pnas.81.13.3944. PMID: 6146135; PMCID: PMC345344.
U.S. Publication No. U.S. Pat. No. 5,122,458, Post et al. (June, 1992).
Orkin S H, Cheng T C, Antonarakis S E, Kazazian H H Jr. Thalassemia due to a mutation in the cleavage-polyadenylation signal of the human beta-globin gene. EMBO J. 1985 February; 4(2):453-6. doi: 10.1002/j.1460-2075.1985.tb03650.x. PMID: 4018033; PMCID: PMC554207.
Thein S L, Wallace R B, Pressley L, Clegg J B, Weatherall D J, Higgs D R. The polyadenylation site mutation in the alpha-globin gene cluster. Blood. 1988 February; 71(2):313-9. PMID: 3337900.
Batt D B, Carmichael G G. Characterization of the polyomavirus late polyadenylation signal. Mol Cell Biol. 1995 September; 15(9):4783-90. doi: 10.1128/MCB.15.9.4783. PMID: 7651395; PMCID: PMC230722.
U.S. Publication No. US 2006/0040354, O'Keefe (February 2006).
Szymanski P, Kretschmer P J, Bauzon M, Jin F, Qian H S, Rubanyi G M, Harkins R N, Hermiston T W. Development and validation of a robust and versatile one-plasmid regulated gene expression system. Mol Ther. 2007 July; 15(7):1340-7. doi: 10.1038/sj.mt.6300171. Epub 2007 May 1. PMID: 17505483.
Schek N, Cooke C, Alwine J C. Definition of the upstream efficiency element of the simian virus 40 late polyadenylation signal by using in vitro analyses. Mol Cell Biol. 1992 December; 12(12):5386-93. doi: 10.1128/mcb.12.12.5386-5393.1992. PMID: 1333042; PMCID: PMC360476.
PCT Publication No. WO 06/12414, O'Kefee (February 2006).
Levitt N, Briggs D, Gil A, Proudfoot N J. Definition of an efficient synthetic poly(A) site. Genes Dev. 1989 July; 3(7):1019-25. doi: 10.1101/gad.3.7.1019. PMID: 2570734.
Banaszynski L A, Chen L C, Maynard-Smith L A, Ooi A G, Wandless T J. A rapid, reversible, and tunable method to regulate protein function in living cells using synthetic small molecules. Cell. 2006 Sep. 8; 126(5):995-1004. doi: 10.1016/j.cell.2006.07.025. PMID: 16959577; PMCID: PMC3290523.
Iwamoto M, Bjorklund T, Lundberg C, Kirik D, Wandless T J. A general chemical method to regulate protein stability in the mammalian central nervous system. Chem Biol. 2010 Sep. 24; 17(9):981-8. doi: 10.1016/j.chembiol.2010.07.009. PMID: 20851347; PMCID: PMC2943492.
U.S. Publication No. U.S. Pat. No. 6,566,118, Atkinson et al. (May 2003).
PCT Publication No. WO 99/11764, Atkinson et al. (March 1999).
PCT Publication No. WO2008135229, Reiter et al. (November 2008).
Grimm D, Kern A, Pawlita M, Ferrari F, Samulski R, Kleinschmidt J. Titration of AAV-2 particles via a novel capsid ELISA: packaging of genomes can limit production of recombinant AAV-2. Gene Ther. 1999 July; 6(7):1322-30. doi: 10.1038/sj.gt.3300946. PMID: 10455443.
Sommer J M, Smith P H, Parthasarathy S, Isaacs J, Vijay S, Kieran J, Powell S K, McClelland A, Wright J F. Quantification of adeno-associated virus particles and empty capsids by optical density measurement. Mol Ther. 2003 January; 7(1):122-8. doi: 10.1016/s1525-0016(02)00019-9. PMID: 12573625.
Wobus C E, Hügle-Dörr B, Girod A, Petersen G, Hallek M, Kleinschmidt J A. Monoclonal antibodies against the adeno-associated virus type 2 (AAV-2) capsid: epitope mapping and identification of capsid domains involved in AAV-2-cell interaction and neutralization of AAV-2 infection. J Virol. 2000 October; 74(19):9281-93. doi: 10.1128/jvi.74.19.9281-9293.2000. PMID: 10982375; PMCID: PMC102127.
M. Lock et al, Hum Gene Ther Methods. 2014 April; 25(2): 15-25. doi: 10.1089/hgtb.2013. 131. Epub 2014 Feb. 14
U.S. Publication No. US 2020/0018752, Bendik et al. (January 2020).
Nguyen T N T, Sha S, Hong M S, Maloney A J, Barone P W, Neufeld C, Wolfrum J, Springs S L, Sinskey A J,
Braatz R D. Mechanistic model for production of recombinant adeno-associated virus via triple transfection of HEK293 cells. Mol Ther Methods Clin Dev (2021) 21:642-655. doi:10.1016/j.omtm.2021.04.006.
Meier A F, Fraefel C, Seyffert M. The Interplay between Adeno-Associated Virus and its Helper Viruses. Viruses (2020) 12: doi:10.3390/v12060662.
Sonntag F, Schmidt K, Kleinschmidt J A. A viral assembly factor promotes AAV2 capsid formation in the nucleolus. Proc Natl Acad Sci USA (2010) 107:10220-10225. doi:10.1073/pnas.1001673107.
Chandler R J, LaFave M C, Varshney G K, Trivedi N S, Carrillo-Carrasco N, Senac J S, Wu W, Hoffmann V, Elkahloun A G, Burgess S M, et al. Vector design influences hepatic genotoxicity after adeno-associated virus gene therapy. J Clin Invest (2015) 125:870-880. doi:10.1172/JCI79213
Gao G, Vandenberghe L H, Alvira M R, Lu Y, Calcedo R, Zhou X, Wilson J M. Clades of Adeno-associated viruses are widely disseminated in human tissues. J Virol (2004) 78:6381-6388. doi:10.1128/JVI.78.12.6381-6388.2004
Song L, Samulski R J, Hirsch M L. Adeno-Associated Virus Vector Mobilization, Risk Versus Reality. Hum Gene Ther (2020) 31:1054-1067. doi:10.1089/hum.2020.118
Robert M-A, Chahal P S, Audy A, Kamen A, Gilbert R, Gaillet B. Manufacturing of recombinant adenoassociated viruses using mammalian expression platforms. Biotechnol J (2017) 12: doi:10.1002/biot.201600193
Li J, Samulski R J, Xiao X. Role for highly regulated rep gene expression in adeno-associated virus vector production. J Virol (1997) 71:5236-5243.
Abdelzaher H M, Gabr A S, Saleh B M, Abdel Gawad R M, Nour A A, Abdelanser A. RNA Vaccines against Infectious Diseases: Vital Progress with Room for Improvement. Vaccines (Basel) (2021) 9:doi:10.3390/vaccines9111211
Baden L R, El Sahly H M, Essink B, Kotloff K, Frey S, Novak R, Diemert D, Spector S A, Rouphael N, Creech C B, et al. Efficacy and Safety of the mRNA-1273 SARS-CoV-2 Vaccine. N Engl J Med (2021) 384:403-416. doi:10.1056/NEJMoa2035389
Fisher K J, Gao G P, Weitzman M D, DeMatteo R, Burda J F, Wilson J M. Transduction with recombinant adeno-associated virus for gene therapy is limited by leading-strand synthesis. J Virol (1996) 70:520-532. doi:10.1128/JVI.70.1.520-532.1996
Chandler R J. Messenger RNA therapy as an option for treating metabolic disorders. Proc Natl Acad Sci USA (2019) 116:20804-20806. doi:10.1073/pnas.1914673116
Ohashi T. Enzyme replacement therapy for lysosomal storage diseases. Pediatr Endocrinol Rev (2012) 10 Suppl 1:26-34.

What is claimed is:

1. A composition comprising a plurality of polynucleotides encoding one or more gene products for production of recombinant virions, wherein at least one of the plurality of polynucleotides are synthetic, and wherein gene products for the production of replication components are comprised on one or more different polynucleotides than gene products for the production of capsid components.

2. The composition of claim 1, wherein the plurality of polynucleotides comprises one or more plasmids.

3. The composition of claim 2, wherein the one or more plasmids comprise a promoter, one or more gene products, one or more regulatory sequence(s), inverted terminal repeats (ITRs), and/or a combination thereof.

4. The composition of claim 3, wherein the one or more regulatory sequence comprises a transcription initiation sequence, an internal ribosome entry site (IRES), an enhancer, an intron, an RNA interference target sequence, a Kozak sequence, splicing regulatory elements, and/or a polyadenylation signal.

5. The composition of claim 3, wherein the promoter is T7.

6. The composition of claim 3, wherein the ITRs are adeno-associated virus (AAV)-derived ITRs.

7. The composition of claim 1, wherein the gene products comprise Rep78, Rep68, Rep52, Rep40, VP1, VP2, VP3, E1A, E1B55K, E2A, E4orf6, assembly activating protein (AAP), viral associated RNA (VA RNA), and/or any combination thereof.

8. The composition of claim 7, wherein the polynucleotide sequence encoding the gene products or encoded polypeptides comprise a sequence at least 80% identical to any one or more of SEQ ID NOs: 1-26 or 40-43.

9. The composition of claim 7, wherein the polynucleotide sequence encoding the gene products or encoded polypeptides comprise a sequence according to any one or more of SEQ ID NOs: 1-26 or 40-43.

10. The composition of claim 1, wherein one or more polynucleotides are ribonucleic acid molecules (RNAs) encoding gene products.

11. The composition of claim 10, wherein the RNAs are produced by chemical synthesis, in vitro transcription, and/or in cellulo.

12. The composition of claim 11, wherein the RNAs comprise modified nucleotides, circularization, and/or mutations relative to wild type sequences.

13. The composition of claim 12, wherein the modified nucleotides comprise pseudouridine and/or 5-methylcytidine.

14. The composition of claim 12, wherein the mutations comprise mutations in the 3′ and/or 5′ untranslated regions (UTRs) that protect the RNAs from degradation.

15. The composition of claim 10, wherein the gene products comprise Rep78, Rep68, Rep52, Rep40, VP1, VP2, VP3, E1A, E1B55K, E2A, E4orf6, assembly activating protein (AAP), viral associated RNA (VA RNA), and/or any combination thereof.

16. The composition of claim 15, wherein the polynucleotide sequence encoding the gene products or encoded polypeptides comprise a sequence at least 80% identical to any one or more of SEQ ID NOs: 1-26 or 40-43.

17. The composition of claim 15, wherein the polynucleotide sequence encoding the gene products or encoded polypeptides comprise a sequence according to any one or more of SEQ ID NOs: 1-26 or 40-43.

18. The composition of claim 10, wherein the RNAs are RNAs purified using chemical-, column-, and/or gel-based approaches.

19. The composition of claim 18, wherein the RNAs are RNAs purified using high performance liquid chromatography (HPLC).

20. A kit comprising the composition of any one of claims 1-19.

21. A composition consisting essentially of RNAs, wherein the RNAs encode gene products for AAV production.

22. The composition of claim 21, wherein the RNAs encode Rep78, Rep68, Rep52, Rep40, VP1, VP2, VP3, E1A, E1B55K, E2A, E4orf6, assembly activating protein (AAP), viral associated RNA (VA RNA), and/or any combination thereof.

23. The composition of claim 22, wherein the RNAs comprise a sequence and/or or encode a polypeptide with at least 80% sequence identity to any one or more of SEQ ID NOs: 1-26 or 40-43.

24. The composition of claim 22, wherein the RNAs comprise a sequence and/or encode a polypeptide according to any one or more of SEQ ID NOs: 1-26 or 40-43.

25. The composition of claim 21, comprising RNAs encoding Rep78, Rep68, Rep52, and/or Rep40.

26. The composition of claim 25, consisting essentially of RNAs encoding Rep78, Rep68, Rep52, and/or Rep40.

27. The composition of claim 21, comprising RNAs encoding VP1, VP2, and/or VP3.

28. The composition of claim 27, consisting essentially of RNAs encoding VP1, VP2, and/or VP3.

29. The composition of claim 21, comprising RNAs encoding E1A, E1B55K, E2A, and/or E4orf6.

30. The composition of claim 29, consisting essentially of RNAs encoding E1A, E1B55K, E2A, and/or E4orf6.

31. The composition of claim 21, comprising RNAs encoding AAP, and/or VA RNA.

32. The composition of claim 31, consisting essentially of RNAs encoding AAP and/or VA RNA.

33. The composition of claim 21, comprising RNAs encoding Rep78, Rep68, Rep52, Rep40, E1A, E1B55K, E2A, and/or E4orf6.

34. The composition of claim 33, consisting essentially of RNAs encoding Rep78, Rep68, Rep52, Rep40, E1A, E1B55K, E2A, and/or E4orf6.

35. The composition of claim 21, comprising RNAs encoding VP1, VP2, VP3, AAP and/or VA RNA.

36. The composition of claim 35, consisting essentially of RNAs encoding VP1, VP2, VP3, AAP and/or VA RNA.

37. A composition comprising the composition of any one claims 21 to 36, and one or more plasmids comprising a promoter sequence, one or more gene product sequence(s), one or more regulatory sequence(s), and inverted terminal repeats (ITRs).

38. The composition of anyone of claims 21 to 36, wherein the RNAs are RNAs produced by chemical synthesis, in vitro transcription, and/or in cellulo.

39. The composition of any one of claims 21 to 36, wherein the RNAs comprise modified nucleotides, circularization, and/or mutations relative to wild type sequences.

40. The composition of claim 39, wherein the modified nucleotides comprise pseudouridine and/or 5-methylcytidine.

41. The composition of claim 39, wherein the mutations comprise mutations in the 3′ or 5′ UTRs that protect the RNAs from degradation.

42. The composition of any one of claims 21 to 36, wherein the RNAs are purified using chemical-, column-, and/or gel-based approaches.

43. The composition of anyone of claims 21 to 36, wherein the RNAs are purified using high performance liquid chromatography (HPLC).

44. A kit comprising the composition of any one of claims 21-36.

45. A method of producing one or more recombinant adeno-associated viruses (rAAVs) comprising performing cell transfection with the compositions of any one of claims 1-19, or 21-36.

46. A method of producing one or more rAAVs comprising one or more temporally distinct cell transfections, wherein the temporally distinct cell transfections may comprise transfection with one or more polynucleotides.

47. The method of claim 46, wherein the polynucleotides comprise a DNA-based recombinant vector, and/or RNA-based viral production genes.

48. The method of claim 47, wherein the DNA-based recombinant vector genome comprises a promoter, one or more gene products, one or more regulatory sequence(s), inverted terminal repeats (ITRs), and/or any combination thereof.

49. The method of claim 48, wherein the promoter is T7.

50. The method of claim 48, wherein the ITRs are adeno-associated virus-derived ITRs.

51. The method of claim 48, wherein the one or more regulatory sequence(s) comprises one or more of, a transcription initiation sequence, an internal ribosome entry site (IRES), an enhancer, an intron, an RNA interference target sequence, a Kozak sequence, splicing regulatory elements, and/or a polyadenylation signal.

52. The method of claim 47, wherein the RNA-based viral production genes comprise rep78, rep68, rep52, rep40, VP1, VP2, VP3, E1A, E1B55K, E2A, E4orf6, AAP, VA RNA, or any combination thereof.

53. The method of claim 52, wherein the RNA-based viral production genes or polypeptide encoded thereby comprise a sequence at least 80% identical to any one or more of SEQ ID NOs:
1-26 or 40-43.

54. The method of claim 53, wherein the RNA-based viral production genes or polypeptide encoded thereby comprise a sequence according to any one or more of SEQ ID NOs: 1-26 or 40-43.

55. The method of any one of claims 46 to 54, comprising the steps of providing to a cell:
a) the DNA-based vector genome;
b) rep genes, comprising rep78, rep68, rep52, rep40, and/or any combination thereof;
c) viral helper genes, comprising E1A, E1B55K, E2A, E4orf6, AAP, VA RNA, and/or any combination thereof;
d) cap genes, comprising VP1, VP2, and VP3, or a combination thereof; and/or
e) viral helper genes, comprising AAP and/or VA RNA.

56. The method of claim 55, wherein step (a), (b), and (c) occur simultaneously.

57. The method of claim 55, wherein step (a), (b), and (c) occur simultaneously, and prior to step (d).

58. The method of claim 55, wherein step (a), (b), and (c) occur simultaneously, and prior to step (d) and (e) which occur simultaneously.

59. The method of claim 57, wherein step (d) and/or (e) occurs 1, 2, 3, 4, 5, or more days after steps (a), (b), and (c).

60. The method of claim 55, wherein each RNA is transfected at a concentration of about 0.1 to 10 μg/100,000 cells.

61. The method of claim 55, wherein each RNA is transfected at a concentration of about 0.1 to 1 μg/100,000 cells.

62. The method of claim 55, wherein the rep genes are transfected at a lower concentration than the viral helper genes and/or cap genes.

63. The method of claim 55, wherein cell transfection comprises use of calcium phosphate, cationic lipids, and polyethyleneimine (PEI), and/or any combination thereof.

64. The method of claim 55, wherein the transfected cells comprise mammalian and/or insect cells.

65. The cells produced according to the method of claim 55.

66. The cells of claim 65, wherein the cells are mammalian cells.

67. The mammalian cells of claim 66, wherein the cells are HeLa, A549, BHK, Vero, 84-32, HEK293, and/or derivate cells thereof.

68. The rAAVs produced according to any one of claims 46 to 54.

69. The rAAVs of claim 68 wherein the virion comprises transcapsidation, mosaicism of the capsid, adsorption of receptor ligands, chimeric and/or hybrid capsids, and/or self-complementarity.

70. The rAAVs of claim 68, wherein the AAVs are purified by clarification of cell supernatant, fractionation, HPLC, and/or any combination thereof.

71. A composition comprising the rAAVs of claim 68, further comprising a pharmaceutically acceptable carrier and/or excipient.

72. A method of providing gene therapy to an individual comprising providing the composition of claim 71.

73. The method of claim 72, wherein the rAAVs comprise an expression construct to provide an individual with a gene product in need thereof.

74. The method of claim 73, wherein the gene product comprises a polypeptide, peptide, antibody, antigen fragment, ribozyme, siRNA, and/or RNAi.

75. The method of claim 72, wherein the compositions are provided to one or more of a cell, organ, and/or tissue of an individual in need thereof.

76. The method of claim 72, wherein the compositions are co-administered with one or more additional therapies.

77. A kit comprising, one or more compositions comprising polynucleotides, wherein the polynucleotides comprise or consisting essentially of:
a) RNAs encoding Rep78, Rep68, Rep52, and/or Rep40;
b) RNAs encoding E1A, E1B55K, E2A, and/or E4orf6;
c) RNAs encoding Rep78, Rep68, Rep52, Rep40, E1A, E1B55K, E2A, and/or E4orf6;
d) RNAs encoding VP1, VP2, and/or VP3;
e) RNAs encoding AAP, and/or VA RNA;
f) RNAs encoding VP1, VP2, VP3, AAP, and/or VA RNA; and/or
g) one or more vector constructs comprising a promoter, one or more gene products, one or more regulatory sequence(s), inverted terminal repeats (ITRs), and/or any combination thereof.

78. The kit of claim 77, wherein the RNAs comprise a sequence, or encode a polypeptide, at least 80% identical to any one or more of SEQ ID NOs: 1-26 or 40-43.

79. The kit of claim 77, wherein the RNAs comprise a sequence, or encode a polypeptide, according to any one or more of SEQ ID NOs: 1-26 or 40-43.


SEQUENCE LISTING (43 sequences)

SEQ ID NO: 1 - protein, 621 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 2 - DNA, 1866 bp, synthetic construct
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 3 - protein, 536 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 4 - DNA, 1932 bp, synthetic construct
  source 1..1932: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 5 - protein, 397 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 6 - DNA, 1194 bp, synthetic construct
  source 1..1194: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 7 - protein, 312 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 8 - DNA, 1260 bp, synthetic construct
  source 1..1260: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 9 - protein, 735 aa, synthetic construct [sequence omitted]

SEQ ID NO: 10 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 11 - protein, 598 aa, synthetic construct
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - DNA, 1797 bp, synthetic construct
  source 1..1797: mol_type = other DNA
acggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 13 - protein, 533 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - DNA, 1602 bp, synthetic construct
  source 1..1602: mol_type = other DNA
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 15 - protein, 204 aa, synthetic construct
METQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 16 - DNA, 615 bp, synthetic construct
  source 1..615: mol_type = other DNA
ctggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctga

SEQ ID NO: 17 - protein, 289 aa, synthetic construct
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 18 - DNA, 984 bp, synthetic construct
  source 1..984: mol_type = other DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtcggtgatagagtggtgggtttggtgtggtaatttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataa

SEQ ID NO: 19 - protein, 495 aa, synthetic construct
MERRNPSERGVPAGFSGHASVESGGETQESPATVVFRPPGNNTDGGATAGGSQAAAAAGAEPMEPESRPGPSGMNVVQVAELFPELRRILTINEDGQGLKGVKRERGASEATEEARNLTFSLMTRHRPECVTFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 20 - DNA, 1488 bp, synthetic construct
  source 1..1488: mol_type = other DNA
atggagcgaagaaacccatctgagcggggggtacctgctggattttctggccatgcatctgtggagagcggtggtgagacacaagaatcgcctgctactgttgtcttccgtccgcccggcaataataccgacggaggagcaacagcaggaggaagccaggcggcggcggcggcaggagcagagcccatggaacccgagagccggcctggaccctcgggaatgaatgttgtacaggtggctgaactgtttccagaactgagacgcattttaaccattaacgaggatgggcaggggctaaagggggtaaagagggagcggggggcttctgaggctacagaggaggctaggaatctaacttttagcttaatgaccagacaccgtcctgagtgtgttacttttcagcagattaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagattagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtgaggtttactggtcccaattttagcggtacggttttcctggccaataccaatcttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctgtttgaaaggtgtaccttgggtatcctgtctgagggtaactccagggtgcgccacaatgtggcctccgactgtggttgcttcatgctagtgaaaagcgtggctgtgattaagcataacatggtgtgtggcaactgcgaggacagggcctctcagatgctgacctgctcggacggcaactgtcacttgctgaagaccattcacgtagccagccactctcgcaaggcctggccagtgtttgagcacaacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 21 - protein, 529 aa, synthetic construct
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 22 - DNA, 1590 bp, synthetic construct
  source 1..1590: mol_type = other DNA
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 23 - protein, 150 aa, synthetic construct
MTTSGVPFGMTLRPTRSRLSRRTPYSRDRLPPFETETRATILEDHPLLPECNTLTMHNAWTSPSPPVEQPQVGQQPVAQQLDSDMNLSELPGEFINITDERLARQETVWNITPKNMSVTHDMMLFKASRGERTVYSVCWEGGGRLNTRVL

SEQ ID NO: 24 - DNA, 1164 bp, synthetic construct
  source 1..1164: mol_type = other DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtga

SEQ ID NO: 25 - DNA, 157 bp, synthetic construct
  source 1..157: mol_type = other DNA
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctcct

SEQ ID NO: 26 - DNA, 158 bp, synthetic construct
  source 1..158: mol_type = other DNA
ggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagcccct

SEQ ID NO: 27 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 28 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - DNA, 255 bp, synthetic construct
  source 1..255: mol_type = other DNA
tcgaggtgagccccacgttctgcttcactctccccatctcccccccctccccacccccaattttgtatttatttattttttaattattttgtgcagcgatgggggcggggggggggggggggcgcgcgccgggggggggggggggggggggggcggggcgaggcggagaggtgcggcggcagccaatcagagcggcgcgctccgaaagtttccttttatggcgaggcggcggcggcggcggccctataaaaagcg

SEQ ID NO: 30 - DNA, 691 bp, synthetic construct
  source 1..691: mol_type = other DNA
tcgaggtgagccccacgttctgcttcactctccccatctcccccccctccccacccccaattttgtatttatttattttttaattattttgtgcagcgatgggggcggggggggggggggggcgcgcgccgggggggggggggggggggggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccacc

SEQ ID NO: 31 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 32 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 33 - protein, 18 aa, synthetic construct
EGRGSLLTCGDVEENPGP

SEQ ID NO: 34 - protein, 19 aa, synthetic construct
ATNFSLLKQAGDVEENPGP

SEQ ID NO: 35 - protein, 20 aa, synthetic construct
QCTNYALLKLAGDVESNPGP

SEQ ID NO: 36 - protein, 22 aa, synthetic construct
VKQTLNFDLLKLAGDVESNPGP

SEQ ID NO: 37 - DNA, 63 bp, synthetic construct
  source 1..63: mol_type = other DNA
ctcgaggccaccaacttctccctgctgaagcaggccggcgacgtggaggagaaccccggcccc

SEQ ID NO: 38 - DNA, 78 bp, synthetic construct
  source 1..78: mol_type = other DNA
ggatcccgggctgactacaaagaccatgacggtgattataaagatcatgacatcgactacaaggatgacgatgacaag

SEQ ID NO: 39 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 40 - protein, 621 aa, synthetic construct; identical to SEQ ID NO: 1

SEQ ID NO: 41 - DNA, 1866 bp, synthetic construct; identical to SEQ ID NO: 2
  source 1..1866: mol_type = other DNA

SEQ ID NO: 42 - protein, 312 aa, synthetic construct; identical to SEQ ID NO: 7

SEQ ID NO: 43 - DNA, 939 bp, synthetic construct
  source 1..939: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacagattggctcgaggacactctctctga